SEQUENCE LISTING (20 sequences)

SEQ ID NO: 1 - DNA, 28 bp, Artificial Sequence
  Description of Artificial Sequence Synthetic primer
ggggtacccttctccaggccagtaggtg

SEQ ID NO: 2 - DNA, 29 bp, Artificial Sequence
  Description of Artificial Sequence Synthetic primer
ggaagatcttggtacagagcgcggacgcg

SEQ ID NO: 3 - DNA, 24 bp, Artificial Sequence
  Description of Artificial Sequence Synthetic primer
ggtaccgccaggcgcctcgacgcc

SEQ ID NO: 4 - DNA, 29 bp, Artificial Sequence
  Description of Artificial Sequence Synthetic primer
ggggtaccggcgagcctataaacaaagcc

SEQ ID NO: 5 - DNA, 36 bp, Artificial Sequence
  Description of Artificial Sequence Synthetic primer
gggcagcggcccgggtaccgtacagtgctcgggcag

SEQ ID NO: 6 - DNA, 36 bp, Artificial Sequence
  Description of Artificial Sequence Synthetic primer
ctgcccgagcactgtacggtacccgggccgctgccc

SEQ ID NO: 7 - DNA, 45 bp, Artificial Sequence
  Description of Artificial Sequence Synthetic primer
tatcgataggtaccggctagcctataagctcagccacgtggcctc

SEQ ID NO: 8 - DNA, 45 bp, Artificial Sequence
  Description of Artificial Sequence Synthetic primer
gaggccacgtggctgagcttataggctagccggtacctatcgata

SEQ ID NO: 9 - DNA, 35 bp, Artificial Sequence
  Description of Artificial Sequence Synthetic oligonucleotide
ctagcaagcaaaacaaacttattttgaacacgggg

SEQ ID NO: 10 - DNA, 35 bp, Artificial Sequence
  Description of Artificial Sequence Synthetic oligonucleotide
ccccgtgttcaaaataagtttgttttgcttgctag

SEQ ID NO: 11 - DNA, 21 bp, Artificial Sequence
  Description of Artificial Sequence Synthetic oligonucleotide
attcgatcggggcggggcgag

SEQ ID NO: 12 - DNA, 21 bp, Artificial Sequence
  Description of Artificial Sequence Synthetic oligonucleotide
ctcgccccgccccgatcgtaa

SEQ ID NO: 13 - DNA, 24 bp, Artificial Sequence
  Description of Artificial Sequence Synthetic oligonucleotide
gggataaatactgtgctcgggcag

SEQ ID NO: 14 - DNA, 24 bp, Artificial Sequence
  Description of Artificial Sequence Synthetic oligonucleotide
ctgcccgagcacagtatttatccc

SEQ ID NO: 15 - DNA, 24 bp, Artificial Sequence
  Description of Artificial Sequence Synthetic oligonucleotide
gggatcactactgtgctcgggcag

SEQ ID NO: 16 - DNA, 24 bp, Artificial Sequence
  Description of Artificial Sequence Synthetic oligonucleotide
ctgcccgagcacagtagtgatccc

SEQ ID NO: 17 - DNA, 82 bp, Artificial Sequence
  Description of Artificial Sequence Synthetic oligonucleotide
tcgaaagtaaacaactatgtaaacaactataagtaaacaactatgtaaacaactataagtaaacaactatgtaaacaagatc

SEQ ID NO: 18 - DNA, 82 bp, Artificial Sequence
  Description of Artificial Sequence Synthetic oligonucleotide
ctaggatcttgtttacatagttgtttacttatagttgtttacatagttgtttacttatagttgtttacatagttgtttactt

SEQ ID NO: 19 - DNA, 19 bp, Artificial Sequence
  Description of Artificial Sequence Synthetic oligonucleotide
ggataagggcgacagcaac

SEQ ID NO: 20 - DNA, 19 bp, Artificial Sequence
  Description of Artificial Sequence Synthetic oligonucleotide
ctggacttccagaagaaca

(19) United States
(12) Patent Application Publication
(10) Pub. No.: US 2006/0069049 A1
(43) Pub. Date: Mar. 30, 2006

(54) Methods and reagents related to foxo

(75) Inventors: Alfred L. Goldberg, Chestnut Hill, MA (US); Stewart H. Lecker, Brookline, MA (US); Marco Sandri, Boston, MA (US)

Correspondence Address: FOLEY HOAG, LLP;PATENT GROUP, WORLD TRADE CENTER WEST, 155 SEAPORT BLVD, BOSTON, MA, 02110, US

(73) Assignee: President and Fellows of Harvard College, Cambridge, MA (US)

(21) Appl. No.: 11/024,371

(22) Filed: Dec. 28, 2004

Related U.S. Application Data
(60) Provisional application No. 60/532,981, filed on Dec. 29, 2003.

(51) Int. Cl. A61K 48/00; C12Q 1/68

(52) U.S. Cl. 514/44; 435/6

(57) ABSTRACT

The present invention relates to regulating the activity of Foxo and prevention and treatment of diseases associated with aberrant Foxo activity.

### CROSS-REFERENCE TO RELATED APPLICATIONS

This application claims the benefit of U.S. Provisional Application No. 60/532,981, filed Dec. 29, 2003, which is hereby incorporated by reference in its entirety.

### GOVERNMENT SUPPORT

This invention was made with government support under grant No. NCC9-58-1 by the National Space Biomedical Research Institute and grant No. K08-DK02707 from the National Institutes of Health. The government has certain rights in the invention.

### BACKGROUND OF THE INVENTION

Muscle atrophy, or muscle wasting, is a highly debilitating response to a wide range of systemic diseases, including cancer cachexia, uremia, AIDS, sepsis, uncontrolled diabetes mellitus, hyperadrenocortisolism (Cushing's Syndrome), trauma, malnutrition, and hyperthyroidism and is also associated with disuse or denervation of muscles. In uremia (renal failure), this excessive breakdown of muscle proteins contributes to the generation of the urea in patients with reduced renal function and reduce ability to dispose of nitrogenous metabolites (urea). Nerve injury, neurodegenerative diseases, and bedrest also cause marked loss of muscle mass and are particularly debilitating clinical problems. These diverse physiological and pathological conditions appear to trigger muscle atrophy through distinct extracellular stimuli, however, the resulting biochemical changes in the atrophying muscles share many common features.

In most conditions when muscles atrophy, overall protein synthesis in muscle is reduced, but the rapid loss of muscle protein results mainly from increased degradation of cell proteins, especially of contractile proteins (myofibrillar proteins), which comprise most of the muscle mass. Furthermore, in all the experimental models of muscle wasting studied thus far, this increased protein degradation results primarily from an activation of the proteolytic system involving ATP, ubiquitin, and the 26S proteasome. We therefore have proposed that in these different catabolic states the atrophying muscles show a common program of changes in gene transcription and activation of common intracellular signaling pathways, which results in cessation of normal growth, an activation of the ubiquitin-proteasome pathway, and net protein loss.

In fact, we have recently characterized the set of transcriptional changes that occur in atrophying muscles and have named these atrophy specific genes “atrogenes”. Among the genes induced in these muscles are polyubiquitin and certain proteasome subunits that support the enhanced rates of proteolysis by the ubiquitin proteasome pathway. In this pathway, proteins are targeted for degradation by linkage to a chain of ubiquitin molecules, which targets the protein for rapid degradation by the 26S proteasome. Formation of the ubiquitin-chain on a protein substrate involves a multienzyme pathway, including E-1 (an ATP-dependent ubiquitin-activating enzyme, and E2 (a ubiquitin carrier protein), and 3 one of cell ubiquitin ligases (E3s) We found that the enzyme that is induced most dramatically in these atrophying muscles is the muscle-specific ubiquitin ligase (E3), atrogin-1. mRNA for atrogin-1 rises 8-40 fold in all types of atrophy studied, and after food deprivation, atrogin-1 mRNA is induced prior to the onset of muscle weight loss. Moreover, knockout animals lacking atrogin-1 show a reduced rate of muscle atrophy after denervation.

A variety of endocrine changes activate protein degradation and trigger systemic muscle wasting. Low levels of insulin, and the resulting decrease in levels of insulin-like growth factor-1 (IGF-1) levels, as well as elevated levels of glucocorticoids, play a major role in the development of muscle protein loss after food deprivation and in diabetes mellitus. Furthermore, insulin resistance appears to be a characteristic feature of systemic diseases such as cancer, uremia and sepsis, and is exacerbated by tumor necrosis factor-α (TNF-α) and glucocorticoid release in these disease states. It seems likely that the diverse stimuli that lead to atrophy act through common signaling mechanisms to influence the same transcription factors. Several recent findings suggest that decreased activity of the insulin-like growth factor-1/phosphoinositide-3 kinase/AKT (IGF-1/PI3K/AKT) signaling pathway can lead to muscle atrophy. We recently used two simple in vitro models of muscle atrophy, cell starvation and dexamethasone treatment, to identify the downstream targets of the IGF1/PI3K/AKT pathway that are important for the induction of the key ubiquitin-protein ligase, atrogin-1, and to the development of muscle wasting.

Herein we describe how IGF-1 acts through AKT to suppress atrogin-1 expression, and that the forkhead family of trascription factors (Foxo1, 3, and 4) activate expression of atrogin-1 and probably other key atrogenes. In particular, we have discovered that Foxo3 acts on the atrogin-1 promoter to trigger expression of this key enzyme, and that overproduction of Foxo3 alone is capable of inducing a decrease in muscle fiber size. In addition, Foxo1 is induced transcriptionally in all atrophy-related conditions. These observations indicate a new and unexpected pathway for development of muscle atrophy—that a decrease in AKT activity leads to activation (dephosphorylation) of Foxo family members, which trigger expression of atrogin-1 and other atrogenes. Moreover, these findings emphasize the key role of Foxo in triggering the program of transcriptional changes in atrophying muscles. Furthermore, Foxo plays an important role in the muscle wasting associated with metabolic diseases. Accordingly, we described herein modulating the expression and activity of Foxo as a means to prevent or reverse the muscle wasting occurring with inactivity or these.

### SUMMARY OF THE INVENTION

The methods and compositions provided herein may be used to treat conditions related to aberrant Foxo activity by modulating the Foxo activity. One aspect of the invention involves treating conditions by modulating Foxo activity by affecting the phosphorylation state of the enzyme. For instance, Foxo activity is induced upon dephosphorylation of the protein. Preferably, maintaining the phosphorylation state of Foxo may be achieved by stimulating the activity of protein kinases, preferably AKT. Conversely, Foxo remains phosphorylated by inhibiting the activity of protein phosphatases, such as protein phospatase 2C.

In another aspect, the invention involves a method for treating a condition involving aberrant Foxo activity by reducing Foxo activity. Foxo activity may be reduced by enhancing it phosphorylation (e.g. by stimulating AKT) or by inhibiting its dephosporylation. Also Foxo activity may be reduced by inhibiting Foxo expression, such as by using anti-sense RNA methods, deletion mutation techniques, or RNAi methods. Preferably, reducing Foxo activity occurs through a dominant negative mutant of Foxo. One example of a dominant negative Foxo mutant lacks the transactivation domain and thus prevents the stimulation of transcription by Foxo.

Still another aspect of the invention involves a diagnostic or prognostic assays for determining, in the context of cells or a muscle biopsy taken from a patient, the level of Foxo phosphorylation, which level can be a useful diagnostic/prognostic marker for risk assessment and phenotyping cell and tissue samples. As described herein, the subject assay provides a method for determining if an animal is at risk for a condition characterized by a metabolic disease or, more preferably, muscle wasting. The subject method can be used for diagnosing a condition involving aberrant Foxo activity in a patient, comprising: (i) ascertaining the level of expression or activity of Foxo; and (ii) diagnosing the presence or absence of a condition involving aberrant Foxo activity utilizing, at least in part, the ascertained level of expression or activity of the Foxo; wherein an increased level of expression or activity of Foxo in the sample, relative to a control sample of non-muscle cells, correlates with the presence of the condition. This assay can also be utilized to optimize the therapeutic efficacy of growth-promoting treatments (e.g. hormones, such as insulin-like growth factor-1 (IGF-1) or novel drugs).

Another aspect of the invention features a method for treating a patient suffering from a condition related to aberrant Foxo activity comprising administering to the patient a compound that promotes the phosphorylation of Foxo or inhibits its dephosphorylation. Alternatively, the patient may receive a gene construct that replaces endogenous Foxo for a dominant negative Foxo mutant, anti-sense RNA for Foxo, or RNA's that interfere with Foxo expression. The method is preferably used to treat patients wherein the condition related to aberrant Foxo activity is associated with cancer cachexia and other muscle wasting conditions, e.g., cachexia secondary to infection or malignancy, cachexia secondary to human acquired immune deficiency syndrome (ADS), AIDS, ARC (ADS related complex); rheumatoid arthritis, cardiac failure, uremia (acidosis), rheumatoid spondylitis, osteoarthritis, gouty arthritis and other arthritic conditions; sepsis, septic shock, endotoxic shock, gram negative sepsis, toxic shock syndrome, adult respiratory distress syndrome, cerebral malaria, chronic pulmonary inflammatory disease, silicosis, pulmonary sarcoidosis, bone resorption diseases, reperfusion injury, graft vs. host reaction, allograft rejections, Crohn's disease, ulcerative colitis, or pyresis, in addition to a number of autoimmune diseases, such as multiple sclerosis, autoimmune diabetes and systemic lupus erythematosis. In addition to treatment of conditions related to aberrant Foxo activity, such inhibitors of atrogin-1 expression could be useful in maintaining muscle mass in bedridden patients, other conditions associated with muscle disuse including patients with traumatic injury, neurodegenerative disease, the aged population which tend to show general sarcopenia (loss of muscle mass), or in space personnel in whom muscle wasting due to the prolonged microgravity environment is a major problem. Inhibitors of activation of the Foxo-family members may also be useful for promoting muscle formation, stimulating proliferation of muscle stem cells, increasing muscle mass, e.g., production of livestock animals. Similarly, genetic modifications in livestock, fowl, fish to prevent the induction of atrogin-1 and other atrogenes by blocking Foxo activity (e.g. by expression of dominant negative inhibitors of Foxo) could generate animals with increased muscle mass, or animals resistant to the costly loss of mass in livestock or horses often seen with febrile illness (e.g. generally termed “shipping fever”).

The present invention relates to a composition of matter comprising a microarray chip containing probes to two or more “atrogenes” that are up- or down-regulated during atrophy related to aberrant Foxo activity.

Still another aspect of the invention pertains to a diagnostic or prognostic method for a conditions related to aberrant Foxo activity involving comprising measuring the up- or down-regulation of two or more genes, such as genes as a part of a microarray set or by real-time PCR.

The methods and compositions provided herein may be used in an assay to identify an agent that promotes the normal activity of Foxo, for example, contacting a cell with a test agent and determining the effect of the test agent on the activity of Foxo. Preferred cells include mammalian cells and more preferably muscle cell lines, such as C2C12, L cells, or human muscle cell lines. A lower activity of Foxo in the presence of the test agent indicates that the agent is particularly useful for preventing or treating conditions involving excess protein degradation and loss of muscle mass. The assay may determine the effect of the agent on the activity or protein level of Foxo. Alternatively, the assay may determine the expression of a reporter gene, such as luciferase or green fluorescent protein, fused with a atrogin-1 promoter after transfection into a cell.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

### BRIEF DESCRIPTION OF THE DRAWINGS

FIG. 1. Starving cells for serum and nutrients and glucocorticoid treatment Induce atrogin-1 expression and dephosphorylation of members of the PI3K/AKT signaling pathway in C2C12 myotubes. a,b. Myotubes were starved by removal of growth medium and incubated in PBS for 6 h. Medium was replaced in refed samples for 12 h. a. Effect of starvation on atrogin-1 expression: Northern blots probed for atrogin-1 and GAPDH (middle panel) and quantitation (upper panel). The fold increase in atrogin-1 mRNA was calculated by dividing the atrogin-1 band intensity (atrogin-1/GADPH) with the atrogin-1/GADPH ratio in the control condition. Results are mean±SEM. Northern panels are representative of at least three sets of experiments performed in duplicate. Lower panel. Micrographs of representative control, starved and refed C2C12 myotube cultures b. Effect of starvation on immunoblots of PI3K/AKT pathway: Proteins were extracted from the same samples analyzed by Northern, and subjected to immunoblot analysis. Densitometric quantitation of the levels of phosphorylated to total protein was determined as above. Results are expressed as mean±SEM. Control data was normalized to 100%. c,d. Myotubes were treated with 1 μM dexamethasone (Dex) for 24 h. c. Effect of dexamethasone treatment on atrogin-1 expression. d. Effect of dexamethasone treatment on immunoblots of PI3K/AKT pathway.

FIG. 2. IGF-1 and AKT block the induction of atrogin-1 by starvation and dexamethasone treatment and cause Foxo phosphorylation. a. Control, starved, and Dex-treated myotubes were incubated in the absence or presence of IGF-1 (10 ng/ml) for 6 hrs (control, starved) and 24 hrs (control, Dex-treated) respectively and analyzed for atrogin-1 expression by Northern blot analysis (upper panel) or amount and phosphorylation of AKT pathway members (lower panel) as in FIG. 1. Northern blot results are the means±SEM of 5 experiments. Immunoblots were also performed on the same cell samples; one representative experiment is shown. b. Myotubes were infected with adenoviral vectors for constitutively active AKT (c.a. AKT), for a dominant-negative AKT (d.n.AKT) and with a control virus (βgal). 36 h after infection, half the myotubes were treated with 1 μM Dex, and atrogin-1 expression was analyzed by Northern as above. Representative examples of atrogin-1 expression are depicted at the bottom. Mean±SEM for atrogin-1 expression was calculated as above from five independent experiments run in duplicate. c. Immunoblots for AKT and its downstream targets in the cultures from b.

FIG. 3. Foxo3 induces atrogin-1 expression and causes reduction in myotube size. a. Foxo3 induces atrogin-1 expression. Myotubes were infected with adenoviral vectors for wild type (FOXO3A) and constitutively active Foxo3 (c.a.FOXO3A) in the absence or presence of IGF-1 (10 ng/ml) and, after 48 h, atrogin-1 mRNA levels were analyzed by Northern blot as already described. Means±SEM for atrogin-1 expression were obtained from three sets of experiments performed in duplicate. Representative examples of atrogin-1 expression under the various conditions are depicted below the quantification. b. The atrogin-1 promoter is activated by Foxo3. Myoblasts were transfected with different atrogin-1 reporter constructs (1.0 AT1, 3.5 AT1) as described in Methods, differentiated and then infected with Ad-FOXO3A or with a control (Ad-GFP) vector for 24 h. Extracts were assayed sequentially for firefly and renilla luciferase activity. Firefly/Renilla activity was normalized to 1.0 in the control (Ad-GFP) infection. Results are means±SEM of five independent experiments. c. Fluorescence microscopy of myotube cultures overexpressing Foxo3. Cultures were infected with control adenovirus (GFP) and constitutively active Foxo3 (c.a.FOXO3A), and photographed 48 h after infection (c.a.FOXO3A also expresses GFP). Mean myotube diameter from each culture was quantified from 160 measurements from three independent experiments. d. A dominant-negative Foxo3 mutant inhibits Dex-induced atrogin-1 expression and reduction in myotube diameter. Myotubes were infected with adenoviral vectors expressing d.n.FOXO3A, c.a.FOXO3A or GFP, and incubated in the absence or presence of 1 μM Dex for 24 hrs. Left panel. Northern analysis of atrogin-1 expression, as described above. Middle panel. Fluorescence microscopy of myotube cultures infected for 48 h with control adenovirus (GFP), adenovirus expressing constitutively active Foxo3 (c.a.FOXO3A), and a dominant-negative Foxo3 mutant (d.nFOXO3A). Right panel. Quantification of mean myotube diameters in the presence of Dex and Foxo expression, as described above. At least 200 measurements for each condition were performed.

FIG. 4. Foxo3, but not other AKT targets, activates atrogin-1 expression. a. Myotubes were infected with various adenoviral vectors for 48 h and then atrogin-1 expression was analyzed by Northern blot as described. (The myotubes infected with wild type Foxo3 (Ad-FOXO3A) were kept in low serum.) Data is representative of at least three independent experiments performed in triplicate. b. Myotubes were infected as above and treated with Dex as described in FIG. 3d. Atrogin-1 expression was analyzed by Northern blot as above. c. Myoblasts were transfected with 3.5 kb atrogin-1 reporter (3.5AT1), differentiated, and infected with the indicated vectors. Luciferase activity in extracts from these cultures were analyzed as in FIG. 3b and Methods. Results are normalized to the control GFP infection.

FIG. 5. AKT suppresses and Foxo stimulates atrogin-1 expression and Foxo activation causes marked atrophy in mouse muscle. a. AKT prevents induction of atrogin-1 expression by fasting. Left panel. Tibialis anterior muscles from CD1 mice were transfected by electroporation with the atrogin-1 reporter and renilla luciferase vector, pRL-TK as described in Methods. 7 days after transfection, the mice were fasted for 24 h and then sacrificed. Muscle extracts were prepared, and compared with extracts obtained from fed control animals for firefly and renilla luciferase activity as described in Methods. Results are the mean ±SEM of six independent experiments. This increase in atrogin-1 reporter activity was confirmed by in situ hybridization on sections from muscles of fed and fasted mice (left panel, insert) (bar: 60 μm). Middle panel: Muscles were cotransfected with the atrogin-1 reporter, pRL-TK and either c.a.HA-AKT or the parent vector as described in Methods. 7 days after transfection, the mice were fasted for 24 hr and sacrificed. Firefly/renilla luciferase activity was measured as above. Results are the mean ±SEM of five independent experiments. Right panel: Serial cross-sections of these transfected muscles were processed for immunofluorescence with anti-HA antibody or for in situ hybridization with an atrogin-1 antisense probe (see Methods). Note that the atrogin-1 transcript is down regulated in the hypertrophied, c.a.HA-AKT-positive fibers (bar: 50 μm). b. Nuclear localization of constitutively active Foxo3. Sections of adult tibialis anterior muscles transfected with HA-tagged constitutively active Foxo3 (c.a.FOXO3A) or wild type Foxo3 (FOXO3A) were prepared and visualized with anti-HA antibodies (for Foxo) and Hoechst staining (for nuclei) 4 days after infection. Images were merged to demonstrate colocalization. Nuclear staining was detected in c.a.FOXO3A-infected fibers, while prominent cytosolic staining was found in Foxo3-overexpressing fibers (bars: 20 μm for c.a.Foxo3a; 30 μm for Foxo3a). c. Foxo3 activates the atrogin-1 promoter in transfected muscle fibers. Muscles were cotransfected with the atrogin-1 reporter and with either FOXO3A or c.a.FOXO3A as described above. In similar experiments, a Foxo reporter (DBE promoter, see Methods) was transfected in place of the atrogin-1 reporter. Luciferase activity was measured as above 4 days after transfection. Results are mean±SEM of six independent experiments. d. Atrogin-1 mRNA is increased in muscle fibers overexpressing Foxo3. Cross-sections of tibialis anterior muscle transfected with c.a.FOXO3A were processed for immunofluorescence with anti-HA antibody (to detect HA-c.a.FOXO3A) or for in situ hybridization for atrogin-1 4 days after transfection, as above. Note that atrogin-1 transcripts are increased in Foxo3 overexpressing fibers, in close proximity to the Foxo3-positive nuclei (arrow) (bar: 40 μm). e. siRNA-mediated inhibition of Foxo1-3 inhibits atrogin-1 promoter activity during fasting. Adult skeletal muscle was cotransfected with pSUPER, pRL-TK and the atrogin-1 reporter as described in the methods. 7 days after transfection, the mice were fasted for 24 hr and sacrificed. Firefly/renilla luciferase activity was measured as above. f. Myofibers expressing Foxo3 are atrophic. Left panel: Adult tibialis anterior muscles were transfected with c.a.FOXO3A and mice were sacrificed after 8 days. Atrophic fibers expressing c.a.FOXO3A are detected in transverse sections stained with anti-HA (for Foxo) (asterix) (bar: 50 μm). Right panel: Frequency histograms showing the distribution of cross-sectional areas (μm²) of fibers expressing c.a.FOXO3A (grey bars) and surrounding untransfected fibers (black bars). The mean±SEM is given for each group. More than 1,800 fibers were analyzed as described in Methods. Adult tibialis anterior muscles were transfected with c.a.FOXO3A and mice were sacrificed after 14 days. Atrophic fibers expressing c.a.FOXO3A are detected in transverse sections stained with anti-HA (bar 20 μm).

FIG. 6. Foxo binding sites are required for atrogin-1 promoter activation by Foxo3. a. Serial truncations of the atrogin-1 promoter were fused the firefly luciferase gene. b. The atrogin-1 reporters and pRL-TK were transfected into adult skeletal muscle in presence or in absence of c.a.FOXO3A as above. Mice were sacrificed four days after transfection and muscles were assayed for luciferase activity as described previously. c. The atrogin-1 promoter. Multiple forkhead consensus binding sites are noted by black circles. Potential forkhead binding site (Foxo₁) present in smallest promoter truncation, used in the gel-shift experiment in d, as well as the mutated version are shown. d. Purified FoxoGST protein was tested for binding to double stranded ³²P-labeled oligonucleotides containing the IGFBP1 site, AT_{Foxo 1} and AT_{Foxo 1mut} sites by electrophoretic mobility shift assay as described in Methods. Arrow: FoxoGST-oligonucleotide complexes. Asterix: nonspecific band. e. left panel. Mutations in the 0.4 kb atrogin-1 luciferase reporter constructs. The two putative Foxo sites are noted by black circles and mutations by X. right panel. Adult skeletal muscle were transfected with the atrogin-1 reporters described in the left panel together with pRL-TK and c.a.FOXO3A as above. Animals were sacrificed and luciferase activity measured as above.

FIG. 7. Roles of the IGF-1/AKT pathway and Foxo in muscle atrophy and hypertrophy. Left panel: Hypertrophying muscle. Right panel: Atrophying muscle. Factors and pathways in bold are activated. See text for details.

FIG. 8. Increased expression of mRNAs involved in protein degradation. Fold increase is graded by intensity of red according to the key. F, Fasting; T, tumor bearing; U, uremia; D, diabetes mellitus.

FIG. 9. MURF-1 expression is increased in atrophying muscles. Total RNA was prepared from the gastrocnemius muscles of control and 2 d (2 day) food-deprived mice, control and rats bearing Yoshida hepatoma for 6 d, control and ⅞ nephrectomized rats, and control and 3 d streptozotocin-treated rats. Northern blot (10 μg total RNA/lane) was performed as in (16). Upper panel: Hybridization with a random-primed probe derived from the full-length MuRF-1 cDNA. Lower panel: Blot stripped and rehybridized with GAPDH cDNA probe to ensure equal RNA loading.

FIG. 10. Differential expression of genes involved in energy production. F, fasting; T, tumor bearing; U, uremia; D, diabetes mellitus. Fold increase is graded by intensity of red and decrease by the intensity of green according to the key.

FIG. 11. Decreased expression of genes involved in transcription and translation. a) Transcription; b) translation. F, Fasting; T, tumor bearing; U, uremia; D, diabetes mellitus.

FIG. 12. Down-regulation of genes encoding extracellular matrix proteins. F, Fasting; T, tumor bearing; U, uremia; D, diabetes mellitus.

FIG. 13. Miscellaneous changes in gene expression in atrophying muscle F, Fasting; T, tumor bearing; U, uremia; D, diabetes mellitus.

FIG. 14. Histogram of the frequency of occurrence of transcription factor binding motifs in up-regulated vs. down-regulated atrogins. For each of the 124 transcription factor binding motifs in the TRANSFECT database, the occurrence frequency in up-regulated vs. down-regulated atrogins was calculated. A ratio of 1:1 would therefore suggest a motif occurring equally in up-and down-regulated genes.

FIG. 15a-g. Representation of Suppmentary Table 2.

### DETAILED DESCRIPTION OF THE INVENTION

1. General

Whether a muscle grows or atrophies depends on the overall balance between its rate of protein synthesis and breakdown. It is now clear that increased protein breakdown is the primary cause of the rapid loss of muscle mass and myofibrillar proteins that occurs upon denervation or disuse and in many systemic diseases, including diabetes, sepsis, hyperthyroidism, cancer cachexia, or fasting. Greater knowledge about the mechanisms that activate proteolysis in muscle is essential if we are to develop rational therapies to combat muscle wasting.

The invention also provides methods for modulating protein degradation, assays for identifying compounds which modulate the accelation of proteolysis, methods for treating disorders associated with excessive protein degradation, diagnostic and prognostic assays for determining whether a subject is at risk of developing a disorder associated with an aberrant protein degradation, or whether therapeutic regimens are working. For example, promoting Foxo phosphorylation or inhibiting Foxo expression could be useful in combating a number of conditions and diseases including cachexia and other muscle wasting, e.g., cachexia secondary to infection or malignancy, cachexia secondary to human acquired immune deficiency syndrome (ADS), AIDS, ARC (ADS related complex); rheumatoid arthritis, rheumatoid spondylitis, osteoarthritis, gouty arthritis and other arthritic conditions; sepsis, septic shock, endotoxic shock, gram negative sepsis, toxic shock syndrome, adult respiratory distress syndrome, cerebral malaria, chronic pulmonary inflammatory disease, silicosis, pulmonary sarcoidosis, bone resorption diseases, reperfusion injury, graft vs. host reaction, allograft rejections, Crohn's disease, ulcerative colitis, or pyresis, in addition to a number of autoimmune diseases, such as multiple sclerosis, autoimmune diabetes and systemic lupus erythematosis. Promoting Foxo phosphorylation is also useful for treatment or prevention of metabolic diseases. In particular, metabolic diseases of the muscle are most likely to benefit from promoting Foxo phosphorylation, which include acid maltase deficiency (Pompe's disease), carnitine deficiency, carnitine palmityl transferase deficiency, debrancher enzyme deficiency (Cori's or Forbes' disease), lactate dehydrogenase deficiency, myoadenylate deaminase deficiency, phosphofructokinase deficiency (Tarui's disease), phosphoglycerate kinase deficiency, phosphoglycerate mutase deficiency and phosphorylase deficiency (McArdle's disease). In addition to treatment of diseases associated with muscle wasting, promoting Foxo phosphorylation or inhibiting Foxo expression could be useful in maintaining muscle mass in bedridden patients or in space personnel in whom muscle wasting due to the prolonged microgravity environment is a major problem. Promoting Foxo phosphorylation or inhibiting Foxo expression may also be useful for promoting muscle formation, stimulating proliferating of muscle stem cells, increasing muscle mass, e.g., production of livestock animals with increased muscle mass, etc.

2. Definitions

For convenience, certain terms employed in the specification, examples, and appended claims are collected here.

The term “aberrant activity”, as applied to an activity of Foxo, refers to an activity which differs from the activity of the wild-type or native form of the protein or because its level of expression is elevated or depressed as compared to the level occurring in a normal cell under normal physiological conditions. The activity of the wild-type or native form of the protein and the expression of Foxo under normal physiological conditions are referred to collectively as “normal activity.” An activity of a protein can be aberrant because it is unregulated, e.g., constitutively activated or inactivated, relative to its normal state. An aberrant activity can also be a change in an activity. For example an aberrant protein can interact with a different protein relative to its native counterpart. A cell can also have an aberrant Foxo activity because of an increase or decrease in Foxo phosphorylation. For instance, aberrant Foxo activity occurs when the cell does not require protein degradation but Foxo remains dephosphorylated and thus stimulates atrogin-1 transcription which induces protein degradation. Conversely, when the cell requires protein degradation, aberrant Foxo activity might occur through Foxo phosphorylation and thus prohibiting the transcription of atrogin-1, which inhibits protein degradation.

As used herein the term “animal” refers to mammals, preferably mammals such as humans.

“Biological activity” or “bioactivity” or “activity” or “biological function”, which are used interchangeably, for the purposes herein means an effector or antigenic function that is directly or indirectly performed by an Foxo polypeptide (whether in its native or denatured conformation), or by any subsequence thereof. Biological activities include binding to the promoter region of a gene, such as atrogin-1. The biological activity of Foxo can also include the ability to promote the degradation of proteins in a muscle cell.

“Cachexia” is the name given to a generally weakened condition of the body or mind resulting from any debilitating chronic disease. The symptoms include severe weight loss, anorexia and anemia. Cachexia is normally associated with neoplasmic diseases, chronic infectious diseases or thyroiditis, and is a particular problem when associated with cancerous conditions.

Indeed, it has been reported that a large proportion of the deaths resulting from cancer are, in fact, associated with cachexia, as also are various other problems commonly experienced by cancer patients, such as respiratory insufficiency, cardiac failure, diseases of the digestive organs, hemorrhaging and systemic infection (U. Cocchi, Strahlentherapie, 69, 503-520 (1941); K. Utsumi et al., Jap. J. Cancer Clinics, 7, 271-283 (1961)).

Cancer associated cachexia, which decreases the tolerance of cancer patients to chemotherapy and radiotherapy is said to be one of the obstacles to effective cancer therapy (J. T. Dwyer, Cancer, 43, 2077-2086 (1979); S. S. Donaldson et al., Cancer, 43, 2036-2052 (1979)). In order to overcome these problems, it used to be common for cancer patients with cachexia to receive a high fat and high sugar diet, or they used to be given high calorie nutrition intravenously. However, it has been reported that symptoms of cachexia were rarely alleviated by these regimens (M. F. Brenann, Cancer Res., 37, 2359-2364 (1977); V. R. Young, Cancer Res., 37, 2336-2347 (1977)).

The phrases “conserved residue” “or conservative amino acid substitution” refer to groupings of amino acids on the basis of certain common properties. A functional way to define common properties between individual amino acids is to analyze the normalized frequencies of amino acid changes between corresponding proteins of homologous organisms (Schulz, G. E. and R. H. Schirmer., Principles of Protein Structure, Springer-Verlag). According to such analyses, groups of amino acids may be defined where amino acids within a group exchange preferentially with each other, and therefore resemble each other most in their impact on the overall protein structure (Schulz, G. E. and R. H. Schirmer., Principles of Protein Structure, Springer-Verlag). Examples of amino acid groups defined in this manner include:
(i) a charged group, consisting of Glu and Asp, Lys, Arg and His,
(ii) a positively-charged group, consisting of Lys, Arg and His,
(iii) a negatively-charged group, consisting of Glu and Asp,
(iv) an aromatic group, consisting of Phe, Tyr and Trp,
(v) a nitrogen ring group, consisting of His and Trp,
(vi) a large aliphatic nonpolar group, consisting of Val, Leu and Ile,
(vii) a slightly-polar group, consisting of Met and Cys,
(viii) a small-residue group, consisting of Ser, Thr, Asp, Asn, Gly, Ala, Glu, Gln and Pro,
(ix) an aliphatic group consisting of Val, Leu, Ile, Met and Cys, and
(x) a small hydroxyl group consisting of Ser and Thr.
   In addition to the groups presented above, each amino acid residue may form its own group, and the group formed by an individual amino acid may be referred to simply by the one and/or three letter abbreviation for that amino acid commonly used in the art.

The term “DNA sequence encoding a polypeptide” may refer to one or more genes within a particular individual. As is well known in the art, genes for a particular polypeptide may exist in single or multiple copies within the genome of an individual. Such duplicate genes may be identical or may have certain modifications, including nucleotide substitutions, additions or deletions, which all still code for polypeptides having substantially the same activity. Moreover, certain differences in nucleotide sequences may exist between individual organisms, which are called alleles. Such allelic differences may or may not result in differences in amino acid sequence of the encoded polypeptide yet still encode a protein with the same biological activity.

The term “domain” as used herein refers to a region within a protein that comprises a particular structure or function different from that of other sections of the molecule.

“Foxo” refers to the members of the forkhead box, class O family of transcription factors, such as Foxo1 (Genbank accession No. NM_{—}019739 and NM_{—}002015), Foxo3 (Genbank accession No. NM_{—}019740 and NP_{—}001446) and Foxo4 (Genbank accession No. Ab032770). Foxo activity is regulated by its phosphorylation status. When phosphorylated by serine/threonine protein kinase Akt/Protein Kinase B (at either threonine 32, serine 253 and/or serine 315 of Foxo3), Foxo is retained in the cytoplasm and has impaired nuclear transcriptional activity. When dephosphorylated, Foxo is translocated to the nucleus and promotes transcriptional activity. The dominant-negative mutant of Foxo3, contains the point mutations wherein threonine 308 is replaced with an alanine and serine 473 is replaced with an alanine. (Datta et al., Genes Dev. 13: 2905-2927 (1999)). Additionally, dominant-negative mutants may comprise nucleic acids and proteins that are significantly identical to the Foxo3 mutants that contain point mutations that inhibit the phosphorylation of Foxo.

“Homology” or “identity” or “similarity” refers to sequence similarity between two peptides or between two nucleic acid molecules. Homology and identity can each be determined by comparing a position in each sequence which may be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology/similarity or identity refers to a function of the number of identical or similar amino acids at positions shared by the compared sequences. A sequence which is “unrelated” or “non-homologous” shares less than 40% identity, though preferably less than 25% identity with a sequence of the present invention.

The term “homology” describes a mathematically based comparison of sequence similarities which is used to identify genes or proteins with similar functions or motifs. The nucleic acid and protein sequences of the present invention may be used as a “query sequence” to perform a search against public databases to, for example, identify other family members, related sequences or homologs. Such searches can be performed using the NBLAST and XBLAST programs (version 2.0) of Altschul, et al. (1990) J Mol. Biol. 215:403-10. BLAST nucleotide searches can be performed with the NBLAST program, score=100, wordlength=12 to obtain nucleotide sequences homologous to nucleic acid molecules of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and BLAST) can be used. See http:/Hwww.ncbi.nlm.nih.gov.

As used herein, “identity” means the percentage of identical nucleotide or amino acid residues at corresponding positions in two or more sequences when the sequences are aligned to maximize sequence matching, i.e., taking into account gaps and insertions. Identity can be readily calculated by known methods, including but not limited to those described in (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48: 1073 (1988). Methods to determine identity are designed to give the largest match between the sequences tested. Moreover, methods to determine identity are codified in publicly available computer programs. Computer program methods to determine identity between two sequences include, but are not limited to, the GCG program package (Devereux, J., et al., Nucleic Acids Research 12(1): 387 (1984)), BLASTP, BLASTN, and FASTA (Altschul, S. F. et al., J. Molec. Biol. 215: 403-410 (1990) and Altschul et al. Nuc. Acids Res. 25: 3389-3402 (1997)). The BLAST X program is publicly available from NCBI and other sources (BLAST Manual, Altschul, S., et al., NCBI NLM NIH Bethesda, Md. 20894; Altschul, S., et al., J. Mol. Biol. 215: 403-410 (1990). The well known Smith Waterman algorithm may also be used to determine identity.

Polypeptides referred to herein as “mammalian homologs” of a protein refers to other mammalian paralogs, or other mammalian orthologs.

The term “motif” as used herein refers to an amino acid sequence that is commonly found in a protein of a particular structure or function. Typically a consensus sequence is defined to represent a particular motif. The consensus sequence need not be strictly defined and may contain positions of variability, degeneracy, variability of length, etc. The consensus sequence may be used to search a database to identify other proteins that may have a similar structure or function due to the presence of the motif in its amino acid sequence. For example, on-line databases such as GenBank or SwissProt can be searched with a consensus sequence in order to identify other proteins containing a particular motif. Various search algorithms and/or programs may be used, including FASTA, BLAST or ENTREZ. FASTA and BLAST are available as a part of the GCG sequence analysis package (University of Wisconsin, Madison, Wis.). ENTREZ is available through the National Center for Biotechnology Information, National Library of Medicine, National Institutes of Health, Bethesda, Md.

The “non-human animals” of the invention include vertebrates such as rodents, non-human primates, sheep, dog, cow, chickens, amphibians, reptiles, etc. Preferred non-human animals are selected from the rodent family including rat and mouse, most preferably mouse, though transgenic amphibians, such as members of the Xenopus genus, and transgenic chickens can also provide important tools for understanding, for example, embryogenesis and tissue patterning. The term “chimeric animal” is used herein to refer to animals in which the recombinant gene is found, or in which the recombinant is expressed in some but not all cells of the animal. The term “tissue-specific chimeric animal” indicates that the recombinant gene is present and/or expressed in some tissues but not others.

As used herein, the term “nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single-stranded (such as sense or antisense) and double-stranded polynucleotides.

The terms peptides, proteins and polypeptides are used interchangeably herein.

The term “purified protein” refers to a preparation of a protein or proteins which are preferably isolated from, or otherwise substantially free of, other proteins normally associated with the protein (s) in a cell or cell lysate. The term “substantially free of other cellular proteins” (also referred to herein as “contaminating proteins”) is defined as encompassing individual preparations of each of the component proteins comprising less than 20% (by dry weight) contaminating protein, and preferably comprises less than 5% contaminating protein. Functional forms of each of the component proteins can be prepared as purified preparations by using a cloned gene. By “purified”, it is meant, when referring to component protein preparations used to generate a reconstituted protein mixture, that the indicated molecule is present in the substantial absence of other biological macromolecules, such as other proteins (particularly other proteins which may substantially mask, diminish, confuse or alter the characteristics of the component proteins either as purified preparations or in their function in the subject reconstituted mixture). The term “purified” as used herein preferably means at least 80% by dry weight, more preferably in the range of 95-99% by weight, and most preferably at least 99.8% by weight, of biological macromolecules of the same type present (but water, buffers, and other small molecules, especially molecules having a molecular weight of less than 5000, can be present). The term “pure” as used herein preferably has the same numerical limits as “purified” immediately above. “Isolated” and “purified” do not encompass either protein in its native state (e.g. as a part of a cell), or as part of a cell lysate, or that have been separated into components (e.g., in an acrylamide gel) but not obtained either as pure (e.g. lacking contaminating proteins) substances or solutions. The term isolated as used herein also refers to a component protein that is substantially free of cellular material or culture medium when produced by recombinant DNA techniques, or chemical precursors or other chemicals when chemically synthesized.

The term “recombinant protein” refers to a protein of the present invention which is produced by recombinant DNA techniques, wherein generally DNA encoding the expressed protein is inserted into a suitable expression vector which is in turn used to transform a host cell to produce the heterologous protein. Moreover, the phrase “derived from”, with respect to a recombinant gene encoding the recombinant protein is meant to include within the meaning of “recombinant protein” those proteins having an amino acid sequence of a native protein, or an amino acid sequence similar thereto which is generated by mutations including substitutions and deletions of a naturally occurring protein.

“Small molecule” as used herein, is meant to refer to a composition, which has a molecular weight of less than about 5 kD and most preferably less than about 2.5 kD. Small molecules can be nucleic acids, peptides, polypeptides, peptidomimetics, carbohydrates, lipids or other organic (carbon containing) or inorganic molecules. Many pharmaceutical companies have extensive libraries of chemical and/or biological mixtures, often fungal, bacterial, or algal extracts, which can be screened with any of the assays of the invention.

As used herein, the term “specifically hybridizes” refers to the ability of a nucleic acid probe/primer of the invention to hybridize to at least 15, 25, 50 or 100 consecutive nucleotides of a target gene sequence, or a sequence complementary thereto, or naturally occurring mutants thereof, such that it has less than 15%, preferably less than 10%, and more preferably less than 5% background hybridization to a cellular nucleic acid (e.g., mRNA or genomic DNA) other than the target gene.

As applied to polypeptides, “substantial sequence identity” means that two mammalian peptide sequences, when optimally aligned, such as by the programs GAP or BESTFIT using default gap which share at least 90 percent sequence identity, preferably at least 95 percent sequence identity, more preferably at least 99 percent sequence identity or more. Preferably, residue positions which are not identical differ by conservative amino acid substitutions. For example, the substitution of amino acids having similar chemical properties such as charge or polarity are not likely to effect the properties of a protein. Examples include glutamine for asparagine or glutamic acid for aspartic acid.

“Transcriptional regulatory sequence” is a generic term used throughout the specification to refer to DNA sequences, such as initiation signals, enhancers, and promoters, which induce or control transcription of protein coding sequences with which they are operably linked. In preferred embodiments, transcription of a recombinant protein gene is under the control of a promoter sequence (or other transcriptional regulatory sequence) which controls the expression of the recombinant gene in a cell-type in which expression is intended. It will also be understood that the recombinant gene can be under the control of transcriptional regulatory sequences which are the same or which are different from those sequences which control transcription of the naturally-occurring form of the protein.

As used herein, the term “vector” refers to a nucleic acid molecule capable of transporting another nucleic acid to which it has been linked. One type of preferred vector is an episome, i.e., a nucleic acid capable of extra-chromosomal replication. Preferred vectors are those capable of autonomous replication and/expression of nucleic acids to which they are linked. Vectors capable of directing the expression of genes to which they are operatively linked are referred to herein as “expression vectors”. In general, expression vectors of utility in recombinant DNA techniques are often in the form of “plasmids” which refer to circular double stranded DNA loops which, in their vector form are not bound to the chromosome. In the present specification, “plasmid” and “vector” are used interchangeably as the plasmid is the most commonly used form of vector. However, the invention is intended to include such other forms of expression vectors which serve equivalent functions and which become known in the art subsequently hereto.

3. Pharmaceutical Compositions and Methods

One embodiment of the invention is an approach to screen for small molecules inhibitors of atrogin-1 expression using the reporter gene constructs, with luciferase fused to an atrogin-1 promoter, such as atrogin-1. Similar reporter gene constructs could be used (e.g. with green fluorescent protein (GFP) instead of luciferase), and transfected into muscle cell lines (C2C12, L cells, or human muscle cell lines) or even into other mammalian cells. This approach can be used to establish lines for use in high throughput screens for small molecules that block atrogin expression. To increase the sensitivity of the screening, the cells may be transfected with wild-type Foxo3 and screen test agents that inactivate Foxo (e.g. increase its phosphorylation, inhibit the dephosphorylation of Foxo-1 in extracts, or otherwixe influence its activity).

To ensure that the test agents are not simply killing cells, one determines the integrity of the cell using standard methods of cell leakage (i.e., lactate dehydrogenase (LDH) release) or measuring protein synthesis (incorporation of radioactive amino acids into proteins). Further, one could check if the test agent inhibits protein loss from the cultured muscle cells, using the dexamethasone treatment or cell starvation methods described in the Examples below. Finally, the drug can be tested to determine if when administered to animals, there is reduced muscle weight loss or cell shrinkage assayed microscopically following denervation, or fasting, or after glucocorticoid treatment of mice or rats.

Pharmaceutical compositions for use in accordance with the present methods may be formulated in conventional manner using one or more physiologically acceptable carriers or excipients. Thus, activating compounds and their physiologically acceptable salts and solvates may be formulated for administration by, for example, injection, inhalation or insufflation (either through the mouth or the nose) or oral, buccal, parenteral or rectal administration. In one embodiment, the compound is administered locally, at the site where the target cells, e.g., diseased cells, are present, i.e., in the blood or in a joint.

Compounds can be formulated for a variety of loads of administration, including systemic and topical or localized administration. Techniques and formulations generally may be found in Remmington's Pharmaceutical Sciences, Meade Publishing Co., Easton, Pa. For systemic administration, injection is preferred, including intramuscular, intravenous, intraperitoneal, and subcutaneous. For injection, the compounds can be formulated in liquid solutions, preferably in physiologically compatible buffers such as Hank's solution or Ringer's solution. In addition, the compounds may be formulated in solid form and redissolved or suspended immediately prior to use. Lyophilized forms are also included.

For oral administration, the pharmaceutical compositions may take the form of, for example, tablets, lozanges, or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., ationd oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate. Preparations for oral administration may be suitably formulated to give controlled release of the active compound.

For administration by inhalation, the compounds may be conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin, for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

Pharmaceutical compositions (including cosmetic preparations) may comprise from about 0.00001 to 100% such as from 0.001 to 10% or from 0.1% to 5% by weight of one or more compounds described herein.

In one embodiment, a compound described herein, is incorporated into a topical formulation containing a topical carrier that is generally suited to topical drug administration and comprising any such material known in the art. The topical carrier may be selected so as to provide the composition in the desired form, e.g., as an ointment, lotion, cream, microemulsion, gel, oil, solution, or the like, and may be comprised of a material of either naturally occurring or synthetic origin. It is preferable that the selected carrier not adversely affect the active agent or other components of the topical formulation. Examples of suitable topical carriers for use herein include water, alcohols and other nontoxic organic solvents, glycerin, mineral oil, silicone, petroleum jelly, lanolin, fatty acids, vegetable oils, parabens, waxes, and the like.

Formulations may be colorless, odorless ointments, lotions, creams, microemulsions and gels.

Compounds may be incorporated into ointments, which generally are semisolid preparations which are typically based on petrolatum or other petroleum derivatives. The specific ointment base to be used, as will be appreciated by those skilled in the art, is one that will provide for optimum drug delivery, and, preferably, will provide for other desired characteristics as well, e.g., emolliency or the like. As with other carriers or vehicles, an ointment base should be inert, stable, nonirritating and nonsensitizing. As explained in Remington's, cited in the preceding section, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin and stearic acid. Exemplary water-soluble ointment bases are prepared from polyethylene glycols (PEGs) of varying molecular weight; again, reference may be had to Remington's, supra, for further information.

Compounds may be incorporated into lotions, which generally are preparations to be applied to the skin surface without friction, and are typically liquid or semiliquid preparations in which solid particles, including the active agent, are present in a water or alcohol base. Lotions are usually suspensions of solids, and may comprise a liquid oily emulsion of the oil-in-water type. Lotions are preferred formulations for treating large body areas, because of the ease of applying a more fluid composition. It is generally necessary that the insoluble matter in a lotion be finely divided. Lotions will typically contain suspending agents to produce better dispersions as well as compounds useful for localizing and holding the active agent in contact with the skin, e.g., methylcellulose, sodium carboxymethylcellulose, or the like. An exemplary lotion formulation for use in conjunction with the present method contains propylene glycol mixed with a hydrophilic petrolatum such as that which may be obtained under the trademark Aquaphor^{RTM} from Beiersdorf, Inc. (Norwalk, Conn.).

Compounds may be incorporated into creams, which generally are viscous liquid or semisolid emulsions, either oil-in-water or water-in-oil. Cream bases are water-washable, and contain an oil phase, an emulsifier and an aqueous phase. The oil phase is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation, as explained in Remington's, supra, is generally a nonionic, anionic, cationic or amphoteric surfactant.

Compounds may be incorporated into microemulsions, which generally are thermodynamically stable, isotropically clear dispersions of two immiscible liquids, such as oil and water, stabilized by an interfacial film of surfactant molecules (Encyclopedia of Pharmaceutical Technology (New York: Marcel Dekker, 1992), volume 9). For the preparation of microemulsions, surfactant (emulsifier), co-surfactant (co-emulsifier), an oil phase and a water phase are necessary. Suitable surfactants include any surfactants that are useful in the preparation of emulsions, e.g., emulsifiers that are typically used in the preparation of creams. The co-surfactant (or “co-emulsifer”) is generally selected from the group of polyglycerol derivatives, glycerol derivatives and fatty alcohols. Preferred emulsifier/co-emulsifier combinations are generally although not necessarily selected from the group consisting of: glyceryl monostearate and polyoxyethylene stearate; polyethylene glycol and ethylene glycol palmitostearate; and caprilic and capric triglycerides and oleoyl macrogolglycerides. The water phase includes not only water but also, typically, buffers, glucose, propylene glycol, polyethylene glycols, preferably lower molecular weight polyethylene glycols (e.g., PEG 300 and PEG 400), and/or glycerol, and the like, while the oil phase will generally comprise, for example, fatty acid esters, modified vegetable oils, silicone oils, mixtures of mono- di- and triglycerides, mono- and di-esters of PEG (e.g., oleoyl macrogol glycerides), etc.

Compounds may be incorporated into gel formulations, which generally are semisolid systems consisting of either suspensions made up of small inorganic particles (two-phase systems) or large organic molecules distributed substantially uniformly throughout a carrier liquid (single phase gels). Single phase gels can be made, for example, by combining the active agent, a carrier liquid and a suitable gelling agent such as tragacanth (at 2 to 5%), sodium alginate (at 2-10%), gelatin (at 2-15%), methylcellulose (at 3-5%), sodium carboxymethylcellulose (at 2-5%), carbomer (at 0.3-5%) or polyvinyl alcohol (at 10-20%) together and mixing until a characteristic semisolid product is produced. Other suitable gelling agents include methylhydroxycellulose, polyoxyethylene-polyoxypropylene, hydroxyethylcellulose and gelatin. Although gels commonly employ aqueous carrier liquid, alcohols and oils can be used as the carrier liquid as well.

Various additives, known to those skilled in the art, may be included in formulations, e.g., topical formulations. Examples of additives include, but are not limited to, solubilizers, skin permeation enhancers, opacifiers, preservatives (e.g., anti-oxidants), gelling agents, buffering agents, surfactants (particularly nonionic and amphoteric surfactants), emulsifiers, emollients, thickening agents, stabilizers, humectants, colorants, fragrance, and the like. Inclusion of solubilizers and/or skin permeation enhancers is particularly preferred, along with emulsifiers, emollients and preservatives. An optimum topical formulation comprises approximately: 2 wt. % to 60 wt. %, preferably 2 wt. % to 50 wt. %, solubilizer and/or skin permeation enhancer; 2 wt. % to 50 wt. %, preferably 2 wt. % to 20 wt. %, emulsifiers; 2 wt. % to 20 wt. % emollient; and 0.01 to 0.2 wt. % preservative, with the active agent and carrier (e.g., water) making of the remainder of the formulation.

A skin permeation enhancer serves to facilitate passage of therapeutic levels of active agent to pass through a reasonably sized area of unbroken skin. Suitable enhancers are well known in the art and include, for example: lower alkanoas such as methanol ethanol and 2-propanol; alkyl methyl sulfoxides such as dimethylsulfoxide (DMSO), decylmethylsulfoxide (C.sub.10 MSO) and tetradecylmethyl sulfboxide; pyrrolidones such as 2-pyrrolidone, N-methyl-2-pyrrolidone and N-(-hydroxyethyl)pyrrolidone; urea; N,N-diethyl-m-toluamide; C.sub.2-C.sub.6 alkanediols; miscellaneous solvents such as dimethyl formamide (DMF), N,N-dimethylacetamide (DMA) and tetrahydrofurfuryl alcohol; and the 1-substituted azacycloheptan-2-ones, particularly 1-n-dodecylcyclazacycloheptan-2-one (laurocapram; available under the trademark Azone^{RTM} from Whitby Research Incorporated, Richmond, Va.).

Examples of solubilizers include, but are not limited to, the following: hydrophilic ethers such as diethylene glycol monoethyl ether (ethoxydiglycol, available commercially as Transcutol^{RTM} ) and diethylene glycol monoethyl ether oleate (available commercially as Softcutol^{RTM} ); polyethylene castor oil derivatives such as polyoxy 35 castor oil, polyoxy 40 hydrogenated castor oil, etc.; polyethylene glycol, particularly lower molecular weight polyethylene glycols such as PEG 300 and PEG 400, and polyethylene glycol derivatives such as PEG-8 caprylic/capric glycerides (available commercially as Labrasol^{RTM} ); alkyl methyl sulfoxides such as DMSO; pyrrolidones such as 2-pyrrolidone and N-methyl-2-pyrrolidone; and DMA. Many solubilizers can also act as absorption enhancers. A single solubilizer may be incorporated into the formulation, or a mixture of solubilizers may be incorporated therein.

Suitable emulsifiers and co-emulsifiers include, without limitation, those emulsifiers and co-emulsifiers described with respect to microemulsion formulations. Emollients include, for example, propylene glycol, glycerol, isopropyl myristate, polypropylene glycol-2 (PPG-2) myristyl ether propionate, and the like.

Other active agents may also be included in formulations, e.g., other anti-inflammatory agents, analgesics, antimicrobial agents, antifungal agents, antibiotics, vitamins, antioxidants, and sunblock agents commonly found in sunscreen formulations including, but not limited to, anthranilates, benzophenones (particularly benzophenone-3), camphor derivatives, cinnamates (e.g., octyl methoxycinnamate), dibenzoyl methanes (e.g., butyl methoxydibenzoyl methane), p-aminobenzoic acid (PABA) and derivatives thereof, and salicylates (e.g., octyl salicylate).

In certain topical formulations, the active agent is present in an amount in the range of approximately 0.25 wt. % to 75 wt. % of the formulation, preferably in the range of approximately 0.25 wt. % to 30 wt. % of the formulation, more preferably in the range of approximately 0.5 wt. % to 15 wt. % of the formulation, and most preferably in the range of approximately 1.0 wt. % to 10 wt. % of the formulation.

Topical skin treatment compositions can be packaged in a suitable container to suit its viscosity and intended use by the consumer. For example, a lotion or cream can be packaged in a bottle or a roll-ball applicator, or a propellant-driven aerosol device or a container fitted with a pump suitable for finger operation. When the composition is a cream, it can simply be stored in a non-deformable bottle or squeeze container, such as a tube or a lidded jar. The composition may also be included in capsules such as those described in U.S. Pat. No. 5,063,507. Accordingly, also provided are closed containers containing a cosmetically acceptable composition as herein defined.

In an alternative embodiment, a pharmaceutical formulation is provided for oral or parenteral administration, in which case the formulation may comprises an activating compound-containing microemulsion as described above, but may contain alternative pharmaceutically acceptable carriers, vehicles, additives, etc. particularly suited to oral or parenteral drug administration. Alternatively, an activating compound-containing microemulsion may be administered orally or parenterally substantially as described above, without modification.

Cells, e.g., treated ex vivo with a compound described herein, can be administered according to methods for administering a graft to a subject, which may be accompanied, e.g., by administration of an immunosuppressant drug, e.g., cyclosporin A. For general principles in medicinal formulation, the reader is referred to Cell Therapy: Stem Cell Transplantation, Gene Therapy, and Cellular Immunotherapy, by G. Morstyn & W. Sheridan eds, Cambridge University Press, 1996; and Hematopoietic Stem Cell Therapy, E. D. Ball, J. Lister & P. Law, Churchill Livingstone, 2000.

4. Therapies Involving Inhibiting Foxo Expression or Activity

Another aspect of the methods and compositions presented herein relates to the use of Foxo nucleic acids in “antisense” therapy. As used herein, “antisense” therapy refers to administration or in situ generation of oligonucleotide molecules or their derivatives which specifically hybridize (e.g., bind) under cellular conditions, with the cellular mRNA and/or genomic DNA encoding one or more of the subject Foxo proteins so as to inhibit expression of that protein, e.g., by inhibiting transcription and/or translation. The binding may be by conventional base pair complementarity, or, for example, in the case of binding to DNA duplexes, through specific interactions in the major groove of the double helix. In general, “antisense” therapy refers to the range of techniques generally employed in the art, and includes any therapy which relies on specific binding to oligonucleotide sequences.

An antisense construct of the methods and compositions presented herein can be delivered, for example, as an expression plasmid which, when transcribed in the cell, produces RNA which is complementary to at least a unique portion of the cellular mRNA which encodes Foxo proteins. Alternatively, the antisense construct is an oligonucleotide probe which is generated ex vivo and which, when introduced into the cell causes inhibition of expression by hybridizing with the mRNA and/or genomic sequences of Foxo genes. Such oligonucleotide probes ate preferably modified oligonucleotides which are resistant to endogenous nucleases, e.g., exonucleases and/or endonucleases, and are therefore stable in vivo. Exemplary nucleic acid molecules for use as antisense oligonucleotides are phosphoramidate, phosphothioate and methylphosphonate analogs of DNA (see also U.S. Pat. Nos. 5,176,996; 5,264,564; and 5,256,775). Additionally, general approaches to constructing oligomers useful in antisense therapy have been reviewed, for example, by Van der Krol et al. (1988) BioTechniques 6:958-976; and Stein et al. (1988) Cancer Res 48:2659-2668. With respect to antisense DNA, oligodeoxyribonucleotides derived from the translation initiation site, e.g., between the −10 and +10 regions of the Foxo nucleotide sequence of interest, are preferred.

Antisense approaches may involve the design of oligonucleotides (either DNA or RNA) that are complementary to Foxo mRNA. The antisense oligonucleotides will bind to Foxo mRNA transcripts and prevent translation. Absolute complementarity, although preferred, is not required. In the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

Oligonucleotides that are complementary to the 5′ end of the mRNA, e.g., the 5′ untranslated sequence up to and including the AUG initiation codon, should work most efficiently at inhibiting translation. However, sequences complementary to the 3′ untranslated sequences of mRNAs have recently been shown to be effective at inhibiting translation of mRNAs as well. (Wagner, R. (1994) Nature 372:333). Therefore, oligonucleotides complementary to either the 5′ or 3′ untranslated, non-coding regions of Foxo genes could be used in an antisense approach to inhibit translation of endogenous Foxo mRNAs. Oligonucleotides complementary to the 5′ untranslated region of the mRNA should include the complement of the AUG start codon. Antisense oligonucleotides complementary to mRNA coding regions are less efficient inhibitors of translation but could also be used in accordance with the methods and compositions presented herein. Whether designed to hybridize to the 5′, 3′ or coding region of Foxo mRNAs, antisense nucleic acids should be at least six nucleotides in length, and are preferably less that about 100 and more preferably less than about 50, 25, 17 or 10 nucleotides in length.

Regardless of the choice of target sequence, it is preferred that in vitro studies are first performed to quantitate the ability of the antisense oligonucleotide to quantitate the ability of the antisense oligonucleotide to inhibit gene expression. In one embodiment these studies utilize controls that distinguish between antisense gene inhibition and nonspecific biological effects of oligonucleotides. In another embodiment these studies compare levels of the target RNA or protein with that of an internal control RNA or protein. Additionally, it is envisioned that results obtained using the antisense oligonucleotide are compared with those obtained using a control oligonucleotide. It is preferred that the control oligonucleotide is of approximately the same length as the test oligonucleotide and that the nucleotide sequence of the oligonucleotide differs from the antisense sequence no more than is necessary to prevent specific hybridization to the target sequence.

The oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors), or agents facilitating transport across the cell membrane (see, e.g., Letsinger et al. (1989) Proc. Natl. Acad. Sci. U.S.A. 86:6553-6556; Lemaitre et al. (1987) Proc. Natl. Acad. Sci. 84:648-652; PCT Publication No. W088/09810, published Dec. 15, 1988) or the blood-brain barrier (see, e.g., PCT Publication No. W089/10134, published Apr. 25, 1988), hybridization-triggered cleavage agents. (See, e.g., Krol et al. (1988) BioTechniques 6:958-976) or intercalating agents. (See, e.g., Zon (1988), Pharm. Res. 5:539-549). To this end, the oligonucleotide may be conjugated to another molecule, e.g., a peptide, hybridization triggered cross-linking agent, transport agent, hybridization-triggered cleavage agent, etc.

The antisense oligonucleotide may comprise at least one modified base moiety which is selected from the group including but not limited to 5-fluorouracil, 5-bromouracil, 5-chlorouracil, 5-iodouracil, hypoxanthine, xantine, 4-acetylcytosine, 5-(carboxyhydroxytiethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methyl guanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

The antisense oligonucleotide may also comprise at least one modified sugar moiety selected from the group including but not limited to arabinose, 2-fluoroarabinose, xylulose, and hexose.

The antisense oligonucleotide can also contain a neutral peptide-like backbone. Such molecules are termed peptide nucleic acid (PNA)-oligomers and are described, e.g., in Perry-O'Keefe et al. (1996) Proc. Natl. Acad. Sci. U.S.A. 93:14670 and in Eglom et al. (1993) Nature 365:566. One advantage of PNA oligomers is their capability to bind to complementary DNA essentially independently from the ionic strength of the medium due to the neutral backbone of the DNA. In yet another embodiment, the antisense oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet a further embodiment, the antisense oligonucleotide is an α-anomenc oligonucleotide. An α-anomeric oligonucleotide forms specific double-stranded hybrids with complementary RNA in which, contrary to the usual b-units, the strands run parallel to each other (Gautier et al. (1987) Nucl. Acids Res. 15:6625-6641). The oligonucleotide is a 2′-0-methylribonucleotide (Inoue et al. (1987) Nucl. Acids Res. 15:6131-6148), or a chimeric RNA-DNA analogue (Inoue et al. (1987) FEBS Lett. 215:327-330).

Oligonucleotides of the methods and compositions presented herein may be synthesized by standard methods known in the art, e.g., by use of an automated DNA synthesizer (such as are commercially available from Biosearch, Applied Biosystems, etc.). As examples, phosphorothioate oligonucleotides may be synthesized by the method of Stein et al. (1988) Nucl. Acids Res. 16:3209, methylphosphonate olgonucleotides can be prepared by use of controlled pore glass polymer supports. (Sarin et al. (1988) Proc. Natl. Acad. Sci. U.S.A. 85:7448-7451), etc.

The antisense molecules can be delivered to cells which express Foxo in vivo. A number of methods have been developed for delivering antisense DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systematically.

A recombinant DNA construct in which the antisense oligonucleotide may be placed under the control of a strong pol III or pol II promoter may also be used. The use of such a construct to transfect target cells in the patient will result in the transcription of sufficient amounts of single stranded RNAs that will form complementary base pairs with the endogenous Foxo transcripts and thereby prevent translation of Foxo mRNAs. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in mammalian, preferably human cells. Such promoters can be inducible or constitutive. Such promoters include but are not limited to: the SV40 early promoter region, (Bernoist et al. (1981) Nature 290:304-310), the promoter-contained in the 3′ long terminal repeat of Rous sarcoma virus (Yamamoto et al. (1980) Cell 22:787-797), the herpes thymidine kinase promoter (Wagner et al. (1981) Proc. Natl. Acad. Sci. U.S.A. 78:1441-1445), the regulatory sequences of the metallothionein gene (Brinster et al. (1982) Nature 296:39-42), etc. Any type of plasmid, cosmid, YAC or viral vector can be used to prepare the recombinant DNA construct which can be introduced directly into the tissue site. Alternatively, viral vectors can be used which selectively infect the desired tissue, in which case administration may be accomplished by another route (e.g., systematically).

Another method for decreasing or blocking gene expression of Foxo is by introducing double stranded small interfering RNAs (siRNAs), which mediate sequence specific mRNA degradation. RNA interference (RNAi) is the process of sequence-specific, post-transcriptional gene silencing in animals and plants, initiated by double-stranded RNA (dsRNA) that is homologous in sequence to the silenced gene. In vivo, long dsRNA is cleaved by ribonuclease III to generate 21- and 22-nucleotide siRNAs. It has been shown that 21-nucleotide siRNA duplexes specifically suppress expression of endogenous and heterologous genes in different mammalian cell lines, including human embryonic kidney (293) and HeLa cells (Elbashir et al. Nature 2001 ;411(6836):494-8). Accordingly, translation of a gene in a cell can be inhibited by contacting the cell with short doublestranded RNAs having a length of about 15 to 30 nucleotides, preferably of about 18 to 21 nucleotides and most preferably 19 to 21 nucleotides. Alternatively, a vector encoding for such siRNAs or hairpin RNAs that are metabolized into siRNAs can be introduced into a target cell (see, e.g., McManus et al. (2002) RNA 8:842; Xia et al. (2002) Nature Biotechnology 20:1006; and Brummelkamp et al. (2002) Science 296:550. Vectors that can be used are commercially available, e.g., from OligoEngine under the name pSuper RNAi System™.

Ribozyme molecules designed to catalytically cleave Foxo mRNA transcripts can also be used to prevent translation of Foxo mRNAs and expression of Foxo polypeptides, or both (See, e.g., PCT International Publication W090/11364, published Oct. 4, 1990; Sarver et al. (1990) Science 247:1222-1225 and U.S. Patent No. 5,093,246). While ribozymes that cleave mRNA at site specific recognition sequences can be used to destroy Foxo mRNAs, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Haseloff and Gerlach (1988) Nature 334:585-591. There are a number of potential hammerhead ribozyme cleavage sites within the nucleotide sequence of human Foxo cDNAs. Preferably the ribozyme is engineered so that the cleavage recognition site is located near the 5′ end of Foxo mRNAs; i.e., to increase efficiency and minimize the intracellular accumulation of non-functional mRNA transcripts.

The ribozymes of the the methods and compositions presented herein also include RNA endoribonucleases (hereinafter “Cech-type ribozymes”) such as the one which occurs naturally in Tetrahymena thermophila (known as the IVS, or L-19 IVS RNA) and which has been extensively described by Thomas Cech and collaborators (Zaug, et al. (1984) Science 224:574-578; Zaug, et al. (1986) Science 231:470475; Zaug, et al. (1986) Nature 324:429-433; published International patent application No. WO88/04300 by University Patents Inc.; Been, et al. (1986) Cell 47:207-216). The Cech-type ribozymes have an eight base pair active site which hybridizes to a target RNA sequence whereafter cleavage of the target RNA takes place. The methods and compositions presented herein encompasses those Cech-type ribozymes which target eight base-pair active site sequences that are present in Foxo genes.

As in the antisense approach, the ribozymes can be composed of modified oligonucleotides (e.g., for improved stability, targeting, etc.) and should be delivered to cells which express Foxo genes in vivo. A preferred method of delivery involves using a DNA construct “encoding” the ribozyme under the control of a strong constitutive pol III or pol II promoter, so that transfected cells will produce sufficient quantities of the ribozyme to destroy endogenous Foxo messages and inhibit translation. Because ribozymes unlike antisense molecules, are catalytic, a lower intracellular concentration is required for efficiency.

Endogenous Foxo gene expression or expression of a splice form thereof can also be reduced by inactivating or “knocking out” Foxo genes or their promoter or a specific exon, using targeted homologous recombination. (E.g., see Smithies et al. (1985) Nature 317:230-234; Thomas, et al. (1987) Cell 51:503-512; Thompson et al. (1989) Cell 5:313-321; each of which is incorporated by reference herein in its entirety). For example, mutant, non-functional Foxo (or a completely unrelated DNA sequence) flanked by DNA homologous to the endogenous Foxo genes (either the coding regions or regulatory regions of Foxo genes) can be used, with or without a selectable marker and/or a negative selectable marker, to transfect cells that express Foxo in vivo. Insertion of the DNA construct, via targeted homologous recombination, results in inactivation of Foxo genes or splice forms thereof. Such approaches are particularly suited in the agricultural field where modifications to ES (embryonic stem) cells can be used to generate animal offspring with an inactive Foxo (e.g., see Thomas, et al. (1987) and Thompson (1989) supra). However this approach can be adapted for use in humans provided the recombinant DNA constructs are directly administered or targeted to the required site in vivo using appropriate viral vectors.

Nucleic acid molecules to be used in triple helix formation for the inhibition of transcription of Foxo genes are preferably single stranded and composed of deoxyribonucleotides. The base composition of these oligonucleotides should promote triple helix formation via Hoogsteen base pairing rules, which generally require sizable stretches of either purines or pyrimidines to be present on one strand of a duplex. Nucleotide sequences may be pyrimidine-based, which will result in TAT and CGC triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, containing a stretch of G residues. These molecules will form a triple helix with a DNA duplex that is rich in GC pairs, in which the majority of the purine residues are located on a single strand of the targeted duplex, resulting in CGC triplets across the three strands in the triplex.

Alternatively, the potential sequences that can be targeted for triple helix formation may be increased by creating a so called “switchback” nucleic acid molecule. Switchback molecules are synthesized in an alternating 5′-3′, 3′-5′ manner, such that they base pair with first one strand of a duplex and then the other, eliminating the necessity for a sizable stretch of either purines or pyrimidines to be present on one strand of a duplex.

Antisense RNA and DNA, ribozyme, and triple helix molecules of the methods and compositions presented herein may be prepared by any method known in the art for the synthesis of DNA and RNA molecules. These include techniques for chemically synthesizing oligodeoxyribonucleotides and oligoribonucleotides well known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

Moreover, various well-known modifications to nucleic acid molecules may be introduced as a means of increasing intracellular stability and half-life. Possible modifications include but are not limited to the addition of flanking sequences of ribonucleotides or deoxyribonucleotides to the 5′ and/or 3′ ends of the molecule or the use of phosphorothioate or 2′ O-methyl rather than phosphodiesterase linkages within the oligodeoxyribonucleotide backbone.

In another embodiment, a nucleic acid encoding a polypeptide of interest, or an equivalent thereof, such as a functionally active fragment of the polypeptide or a dominant negative fragment of the polypeptide, is administered to a subject, such that the nucleic acid arrives at the site of the diseased cells, traverses the cell membrane and is expressed in the diseased cell.

Any means for the introduction of polynucleotides into mammals, human or non-human, may be adapted to the practice of this invention for the delivery of the various constructs of the invention into the intended recipient. In one embodiment of the invention, the DNA constructs are delivered to cells by transfection, i.e., by delivery of “naked” DNA or in a complex with a colloidal dispersion system. A colloidal system includes macromolecule complexes, nanocapsules, microspheres, beads, and lipid-based systems including oil-in-water emulsions, micelles, mixed micelles, and liposomes. The preferred colloidal system of this invention is a lipid-complexed or liposome-formulated DNA. In the former approach, prior to formulation of DNA, e.g., with lipid, a plasmid containing a transgene bearing the desired DNA constructs may first be experimentally optimized for expression (e.g., inclusion of an intron in the 5′ untranslated region and elimination of unnecessary sequences (Felgner, et al., Ann. N.Y. Acad. Sci. 126-139, 1995). Formulation of DNA, e.g. with various lipid or liposome materials, may then be effected using known methods and materials and delivered to the recipient mammal. See, e.g., Canonico et al, Am. J. Respir. Cell. Mol. Biol. 10:24-29, 1994; Tsan et al, Am. J. Physiol. 268; Alton et al., Nat. Genet. 5:135-142, 1993 and U.S. Pat. No. 5,679,647 by Carson et al.

The targeting of liposomes can be classified based on anatomical and mechanistic factors. Anatomical classification is based on the level of selectivity, for example, organ-specific, cell-specific, and organelle-specific. Mechanistic targeting can be distinguished based upon whether it is passive or active. Passive targeting utilizes the natural tendency of liposomes to distribute to cells of the reticulo-endothelial system (RES) in organs, which contain sinusoidal capillaries. Active targeting, on the other hand, involves alteration of the liposome by coupling the liposome to a specific ligand such as a monoclonal antibody, sugar, glycolipid, or protein, or by changing the composition or size of the liposome in order to achieve targeting to organs and cell types other than the naturally occurring sites of localization.

The surface of the targeted delivery system may be modified in a variety of ways. In the case of a liposomal targeted delivery system, lipid groups can be incorporated into the lipid bilayer of the liposome in order to maintain the targeting ligand in stable association with the liposomal bilayer. Various linking groups can be used for joining the lipid chains to the targeting ligand. Naked DNA or DNA associated with a delivery vehicle, e.g., liposomes, can be administered to several sites in a subject (see below).

In another method, the DNA constructs are delivered using viral vectors. The transgene may be incorporated into any of a variety of viral vectors useful in gene therapy, such as recombinant retroviruses, adenovirus, adeno-associated virus (AAV), and herpes simplex virus-1, or recombinant bacterial or eukaryotic plasmids. While various viral vectors may be used in the practice of this invention, AAV- and adenovirus-based approaches are of particular interest. Such vectors are generally understood to be the recombinant gene delivery system of choice for the transfer of exogenous genes in vivo, particularly into humans.

It is possible to limit the infection spectrum of viruses by modifying the viral packaging proteins on the surface of the viral particle (see, for example PCT publications WO93/25234, WO94/06920, and WO94/11524). For instance, strategies for the modification of the infection spectrum of viral vectors include: coupling antibodies specific for cell surface antigens to envelope protein (Roux et al., (1989) PNAS USA 86:9079-9083; Julan et al., (1992) J. Gen Virol 73:3251-3255; and Goud et al., (1983) Virology 163:251-254); or coupling cell surface ligands to the viral envelope proteins (Neda et al., (1991) J. Biol. Chem. 266:14143-14146). Coupling can be in the form of the chemical cross-linking with a protein or other variety (e.g. lactose to convert the env protein to an asialoglycoprotein), as well as by generating fusion proteins (e.g. single-chain antibody/env fusion proteins). This technique, while useful to limit or otherwise direct the infection to certain tissue types, and can also be used to convert an ecotropic vector in to an amphotropic vector.

The expression of or inhibition of the expression of a polypeptide of interest in cells of a patient to which a nucleic acid encoding the polypeptide or inhibiting expression was administered can be determined, e.g., by obtaining a sample of the cells of the patient and determining the level of the polypeptide in the sample, relative to a control sample.

5. Introduction: Microarray

Generally, determining expression profiles with arrays involves the following steps: (a) obtaining a mRNA sample from a subject and preparing labeled nucleic acids therefrom (the “target nucleic acids” or “targets”); (b) contacting the target nucleic acids with the array under conditions sufficient for target nucleic acids to bind with corresponding probes on the array, e.g. by hybridization or specific binding; (c) optionally removing unbound targets from the array; (d) detecting bound targets, and (e) analyzing the results. As used herein, “nucleic acid probes” or “probes” are nucleic acids attached to the array, whereas “target nucleic acids” are nucleic acids that are hybridized to the array. Each of these steps is described in more detail below.

5.1 Labeling the Nucleic Acid for the Microarray Analysis

Generally, the target molecules will be labeled to permit detection of hybridization of target molecules to a microarray. By “labeled” is meant that the probe comprises a member of a signal producing system and is thus detectable, either directly or through combined action with one or more additional members of a signal producing system. Examples of directly detectable labels include isotopic and fluorescent moieties incorporated into, usually covalently bonded to, a moiety of the probe, such as a nucleotide monomeric unit, e.g. dNMP of the primer, or a photoactive or chemically active derivative of a detectable label which can be bound to a functional moiety of the probe molecule.

Nucleic acids can be labeled after or during enrichment and/or amplification of RNAs. For example, labeled cDNA can be prepared from mRNA by oligo dT-primed or random-primed reverse transcription, both of which are well known in the art (see, e.g., Klug and Berger, 1987, Methods Enzymol. 152:316-325). Reverse transcription may be carried out in the presence of a dNTP conjugated to a detectable label, most preferably a fluorescently labeled dNTP. Alternatively, isolated mRNA can be converted to labeled antisense RNA synthesized by in vitro transcription of double-stranded cDNA in the presence of labeled dNTPs (Lockhart et al., Nature Biotech. 14:1675, 1996). In alternative embodiments, the cDNA or RNA probe can be synthesized in the absence of detectable label and may be labeled subsequently, e.g., by incorporating biotinylated dNTPs or rNTP, or some similar means (e.g., photo-cross-linking a psoralen derivative of biotin to RNAs), followed by addition of labeled streptavidin (e.g., phycoerythrin-conjugated streptavidin) or the equivalent.

In one embodiment, labeled cDNA is synthesized by incubating a mixture containing RNA and 0.5 mM dGTP, dATP and dCTP plus 0.1 mM dTTP plus fluorescent deoxyribonucleotides (e.g., 0.1 mM Rhodamine 110 UTP (Perken Elmer Cetus) or 0.1 mM Cy3 dUTP (Amersham)) with reverse transcriptase (e.g., SuperScript™II, LTI Inc.) at 42° C., for 60 min.

Fluorescent moieties or labels of interest include coumarin and its derivatives, e.g. 7-amino-4-methylcoumarin, aminocoumarin, bodipy dyes, such as Bodipy FL, cascade blue, fluorescein and its derivatives, e.g. fluorescein isothiocyanate, Oregon green, rhodamine dyes, e.g. Texas red, tetramethylrhodamine, eosins and erythrosins, cyanine dyes, e.g. Cy2, Cy3, Cy3.5, Cy5, Cy5.5, Cy7, FluorX, macrocyclic chelates of lanthanide ions, e.g. quantum dye™, fluorescent energy transfer dyes, such as thiazole orange-ethidium heterodimer, TOTAB, dansyl, etc. Individual fluorescent compounds which have functionalities for linking to an element desirably detected in an apparatus or assay of the invention, or which can be modified to incorporate such functionalities include, e.g., dansyl chloride; fluoresceins such as 3,6-dihydroxy-9-phenylxanthydrol; rhodamineisothiocyanate; N-phenyl 1-amino-8-sulfonatonaphthalene; N-phenyl 2-amino-6-sulfonatonaphthalene; 4-acetamido-4-isothiocyanato-stilbene-2,2′-disulfonic acid; pyrene-3-sulfonic acid; 2-toluidinonaphthalene-6-sulfonate; N-phenyl-N-methyl-2-aminoaphthalene-6-sulfonate; ethidium bromide; stebrine; auromine-0,2-(9′-anthroyl)palmitate; dansyl phosphatidylethanolamine; N,N′-dioctadecyl oxacarbocyanine: N,N′-dihexyl oxacarbocyanine; merocyanine, 4-(3′-pyrenyl)stearate; d-3-aminodesoxy-equilenin; 12-(9′-anthroyl)stearate; 2-methylanthracene; 9-vinylanthracene; 2,2′(vinylene-p-phenylene)bisbenzoxazole; p-bis(2-methyl-5-phenyl-oxazolyl))benzene; 6-dimethylamino-1,2-benzophenazin; retinol; bis(3′-aminopyridinium) 1,10-decandiyl diiodide; sulfonaphthylhydrazone of hellibrienin; chlorotetracycline; N-(7-dimethylamino-4-methyl-2-oxo-3-chromenyl)maleimide; N-(p-(2benzimidazolyl)-phenyl)maleimide; N-(4-fluoranthyl)maleimide; bis (homovanillic acid); resazarin; 4-chloro-7-nitro-2,1,3-benzooxadiazole; merocyanine 540; resorufin; rose bengal; and 2,4-diphenyl-3(2H)-furanone. (see, e.g., Kricka, 1992, Nonisotopic DNA Probe Techniques, Academic Press San Diego, Calif.). Many fluorescent tags are commercially available from SIGMA-Aldrich, Amersham Biosciences, Molecular Probes, Pfizer (formerly Pharmacia), BD Biosciences (formerly CLONTECH), ChemGenes Corp., Glen Research Corp., Invitrogen, Fluka Chemica-Biochemika Analytika (Fluka Chemie AG, Buchs, Switzerland), and Applied Biosystems (Foster City, Calif.) as well as other commercial sources known to one of skill.

Chemiluminescent labels include luciferin and 2,3-dihydrophthalazinediones, e.g., luminol.

Isotopic moieties or labels of interest include ³²P, ³³P, ³⁵S, ¹²⁵I, ²H, ¹⁴C, and the like (see Zhao et al., Gene 156:207, 1995; Pietu et al., Genome Res. 6:492, 1996).

Labels may also be members of a signal producing system that act in concert with one or more additional members of the same system to provide a detectable signal. Illustrative of such labels are members of a specific binding pair, such as ligands, e.g. biotin, fluorescein, digoxigenin, antigen, polyvalent cations, chelator groups and the like, where the members specifically bind to additional members of the signal producing system, where the additional members provide a detectable signal either directly or indirectly, e.g. antibody conjugated to a fluorescent moiety or an enzymatic moiety capable of converting a substrate to a chromogenic product, e.g. alkaline phosphatase conjugate antibody and the like.

Additional labels of interest include those that provide for signal only when the probe with which they are associated is specifically bound to a target molecule, where such labels include: “molecular beacons” as described in Tyagi & Kramer, Nature Biotechnology 14:303, 1996 and EP 0 070 685 B1. Other labels of interest include those described in U.S. Pat. No. 5,563,037; WO 97/17471 and WO 97/17076.

In some cases, hybridized target nucleic acids may be labeled following hybridization. For example, where biotin labeled dNTPs are used in, e.g., amplification or transcription, streptavidin linked reporter groups may be used to label hybridized complexes.

In other embodiments, the target nucleic acid is not labeled. In this case, hybridization can be determined, e.g., by plasmon resonance, as described, e.g., in Thiel et al., Anal. Chem. 69:4948, 1997.

In one embodiment, a plurality (e.g., 2, 3, 4, 5 or more) of sets of target nucleic acids are labeled and used in one hybridization reaction (“multiplex” analysis). For example, one set of nucleic acids may correspond to RNA from one cell or tissue sample and another set of nucleic acids may correspond to RNA from another cell or tissue sample. The plurality of sets of nucleic acids can be labeled with different labels, e.g., different fluorescent labels which have distinct emission spectra so that they can be distinguished. The sets can then be mixed and hybridized simultaneously to one microarray.

The use of a two-color fluorescence labeling and detection scheme to define alterations in gene expression has been described, e.g., in Shena et al., Science 270:467-470, 1995. An advantage of using cDNA labeled with two different fluorophores is that a direct and internally controlled comparison of the mRNA levels corresponding to each arrayed gene in two cell states can be made, and variations due to minor differences in experimental conditions (e.g. hybridization conditions) will not affect subsequent analyses.

Examples of distinguishable labels for use when hybridizing a plurality of target nucleic acids to one array are well known in the art and include: two or more different emission wavelength fluorescent dyes, like Cy3 and Cy5, combination of fluorescent proteins and dyes, like phicoerythrin and Cy5, two or more isotopes with different energy of emission, like ³²P and ³³P, gold or silver particles with different scattering spectra, labels which generate signals under different treatment conditions, like temperature, pH, treatment by additional chemical agents, etc., or generate signals at different time points after treatment. Using one or more enzymes for signal generation allows for the use of an even greater variety of distinguishable labels, based on different substrate specificity of enzymes (alkaline phosphatase/peroxidase).

Further, it is preferable in order to reduce experimental error to reverse the fluorescent labels in two-color differential hybridization experiments to reduce biases peculiar to individual genes or array spot locations. In other words, it is preferable to first measure gene expression with one labeling (e.g., labeling nucleic acid from a first cell with a first fluorochrome and nucleic acid from a second cell with a second fluorochrome) of the mRNA from the two cells being measured, and then to measure gene expression from the two cells with reversed labeling (e.g., labeling nucleic acid from the first cell with the second fluorochrome and nucleic acid from the second cell with the first fluorochrome). Multiple measurements over exposure levels and perturbation control parameter levels provide additional experimental error control.

The quality of labeled nucleic acids can be evaluated prior to hybridization to an array. For example, a sample of the labeled nucleic acids can be hybridized to probes derived from the 5′, middle and 3′ portions of genes known to be or suspected to be present in the nucleic acid sample. This will be indicative as to whether the labeled nucleic acids are full length nucleic acids or whether they are degraded. In one embodiment, the GeneChip® Test3 Array from Affymetrix (Santa Clara, Calif.) can be used for that purpose. This array contains probes representing a subset of characterized genes from several organisms including mammals. Thus, the quality of a labeled nucleic acid sample can be determined by hybridization of a fraction of the sample to an array, such as the GeneChip® Test3 Array from Affymetrix (Santa Clara, Calif.).

5.2 Microarray Analysis

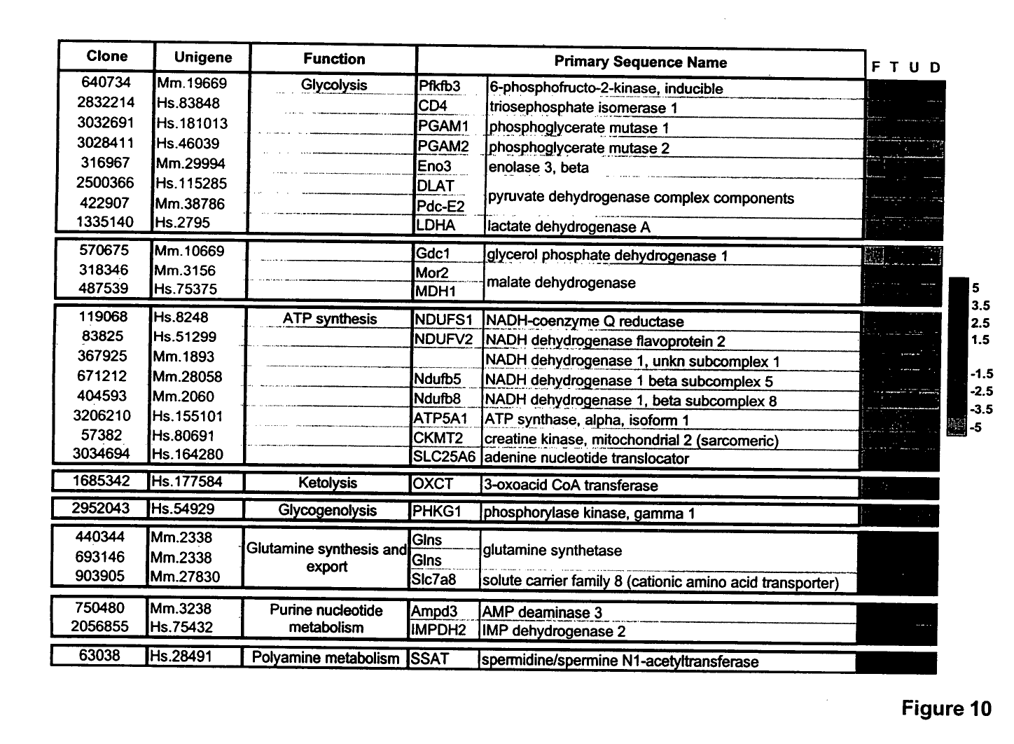

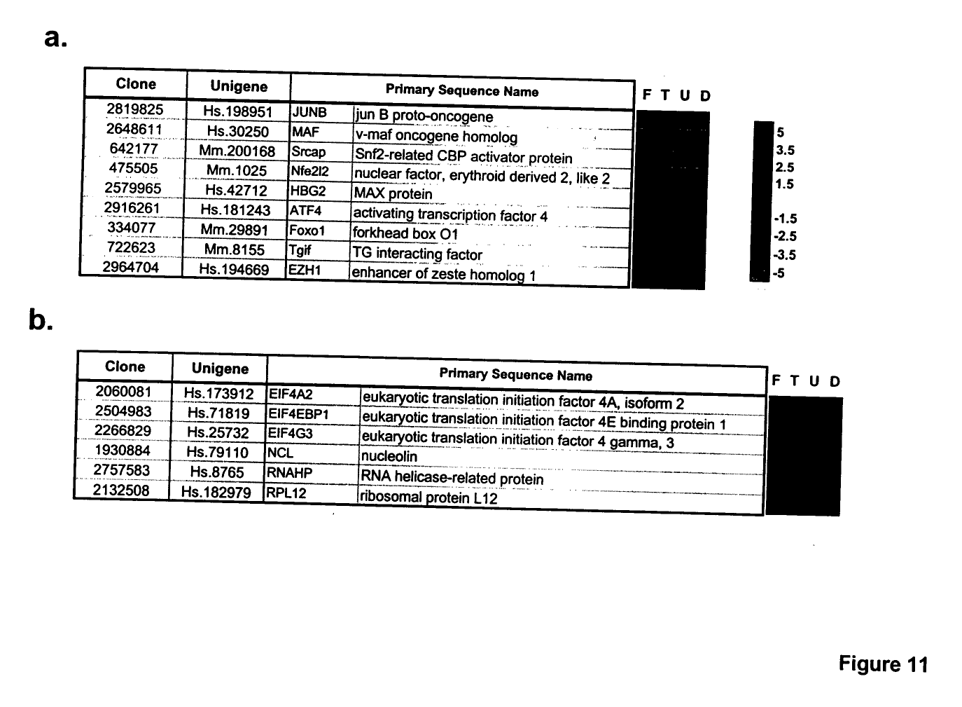

The array may comprise probes corresponding to at least 10, preferably at least 20, at least 50, at least 100 or at least 1000 genes. The array may comprise probes corresponding to about 10%, 20%, 50%, 70%, 90% or 95% of the genes listed in FIG. 10 or other genes available on a microarray. The array may comprise probes corresponding to about 10%, 20%, 50%, 70%, 90% or 95% of the genes listed in Example 1 or other gene whose expression is at least 2 fold, preferably at least 3 fold, more preferably at least 4 fold, 5 fold, 7 fold and most preferably at least about 10 fold higher in cells. One exemplary preferred array that can be used is the array used and described in Example 1.

There can be one or more than one probe corresponding to each gene on a microarray. For example, a microarray may contain from 2 to 20 probes corresponding to one gene and preferably about 5 to 10. The probes may correspond to the full length RNA sequence or complement thereof of genes characteristic of candidate disease genes, or they may correspond to a portion thereof, which portion is of sufficient length for permitting specific hybridization. Such probes may comprise from about 50 nucleotides to about 100, 200, 500, or 1000 nucleotides or more than 1000 nucleotides. As further described herein, microarrays may contain oligonucleotide probes, consisting of about 10 to 50 nucleotides, preferably about 15 to 30 nucleotides and even more preferably 20-25 nucleotides. The probes are preferably single stranded. The probe will have sufficient complementarity to its target to provide for the desired level of sequence specific hybridization (see below).

Typically, the arrays used in the present invention will have a site density of greater than 100 different probes per cm2. Preferably, the arrays will have a site density of greater than 500/cm2, more preferably greater than about 1000/cm2, and most preferably, greater than about 10,000/cm2. Preferably, the arrays will have more than 100 different probes on a single substrate, more preferably greater than about 1000 different probes still more preferably, greater than about 10,000 different probes and most preferably, greater than 100,000 different probes on a single substrate.

Microarrays can be prepared by methods known in the art, as described below, or they can be custom made by companies, e.g., Affymetrix (Santa Clara, Calif.).

Generally, two types of microarrays can be used. These two types are referred to as “synthesis” and “delivery.” In the synthesis type, a microarray is prepared in a step-wise fashion by the in situ synthesis of nucleic acids from nucleotides. With each round of synthesis, nucleotides are added to growing chains until the desired length is achieved. In the delivery type of microarray, preprepared nucleic acids are deposited onto known locations using a variety of delivery technologies. Numerous articles describe the different microarray technologies, e.g., Shena et al., Tibtech 16: 301, 1998; Duggan et al., Nat. Genet. 21:10, 1999; Bowtell et al., Nat. Genet. 21: 25, 1999.

Arrays preferably include control and reference nucleic acids. Control nucleic acids are nucleic acids which serve to indicate that the hybridization was effective. For example, all Affymetrix (Santa Clara, Calif.) expression arrays contain sets of probes for several prokaryotic genes, e.g., bioB, bioC and bioD from biotin synthesis of E. coli and cre from P1 bacteriophage. Hybridization to these arrays is conducted in the presence of a mixture of these genes or portions thereof, such as the mix provided by Affymetrix (Santa Clara, Calif.) to that effect (Part Number 900299), to thereby confirm that the hybridization was effective. Control nucleic acids included with the target nucleic acids can also be mRNA synthesized from cDNA clones by in vitro transcription. Other control genes that may be included in arrays are polyA controls, such as dap, lys, phe, thr, and trp (which are included on Affymetrix GeneChips®)

Reference nucleic acids allow the normalization of results from one experiment to another, and to compare multiple experiments on a quantitative level. Exemplary reference nucleic acids include housekeeping genes of known expression levels, e.g., glyceraldehyde-3-phosphate dehydrogenase (GAPDH), hexokinase and actin.

Mismatch controls may also be provided for the probes to the target genes, for expression level controls or for normalization controls. Mismatch controls are oligonucleotide probes or other nucleic acid probes identical to their corresponding test or control probes except for the presence of one or more mismatched bases.

Arrays may also contain probes that hybridize to more than one allele of a gene. For example the array can contain one probe that recognizes allele 1 and another probe that recognizes allele 2 of a particular gene.

Microarrays can be prepared as follows. In one embodiment, an array of oligonucleotides is synthesized on a solid support. Exemplary solid supports include glass, plastics, polymers, metals, metalloids, ceramics, organics, etc. Using chip masking technologies and photoprotective chemistry it is possible to generate ordered arrays of nucleic acid probes. These arrays, which are known, e.g., as “DNA chips,” or as very large scale immobilized polymer arrays (“VLSIPSTM” arrays) can include millions of defined probe regions on a substrate having an area of about 1 cm² to several cm², thereby incorporating sets of from a few to millions of probes (see, e.g., U.S. Pat. No. 5,631,734).

The construction of solid phase nucleic acid arrays to detect target nucleic acids is well described in the literature. See, Fodor et al., Science, 251: 767-777, 1991; Sheldon et al., Clinical Chemistry 39(4): 718-719, 1993; Kozal et al., Nature Medicine 2(7): 753-759, 1996 and Hubbell U.S. Pat. No. 5,571,639; Pinkel et al. PCT/US95/16155 (WO 96/17958); U.S. Pat. Nos. 5,677,195; 5,624,711; 5,599,695; 5,451,683; 5,424,186; 5,412,087; 5,384,261; 5,252,743 and 5,143,854; PCT Patent Publication Nos. 92/10092 and 93/09668; and PCT WO 97/10365. In brief, a combinatorial strategy allows for the synthesis of arrays containing a large number of probes using a minimal number of synthetic steps. For instance, it is possible to synthesize and attach all possible DNA 8 mer oligonucleotides (48, or 65,536 possible combinations) using only 32 chemical synthetic steps. In general, VLSIPSTM procedures provide a method of producing 4 n different oligonucleotide probes on an array using only 4 n synthetic steps (see, e.g., U.S. Pat. Nos. 5,631,734, 5,143,854 and PCT Patent Publication Nos. WO 90/15070; WO 95/11995 and WO 92/10092).

Light-directed combinatorial synthesis of oligonucleotide arrays on a glass surface can be performed with automated phosphoramidite chemistry and chip masking techniques similar to photoresist technologies in the computer chip industry. Typically, a glass surface is derivatized with a silane reagent containing a functional group, e.g., a hydroxyl or amine group blocked by a photolabile protecting group. Photolysis through a photolithogaphic mask is used selectively to expose functional groups which are then ready to react with incoming 5′-photoprotected nucleoside phosphoramidites. The phosphoramidites react only with those sites which are illuminated (and thus exposed by removal of the photolabile blocking group). Thus, the phosphoramidites only add to those areas selectively exposed from the preceding step. These steps are repeated until the desired array of sequences have been synthesized on the solid surface.

Algorithms for design of masks to reduce the number of synthesis cycles are described by Hubbel et al., U.S. Pat. No. 5,571,639 and U.S. Pat. No. 5,593,839. A computer system may be used to select nucleic acid probes on the substrate and design the layout of the array as described in U.S. Pat. No. 5,571,639.

Another method for synthesizing high density arrays is described in U.S. Pat. No. 6,083,697. This method utilizes a novel chemical amplification process using a catalyst system which is initiated by radiation to assist in the synthesis the polymer sequences. Such methods include the use of photosensitive compounds which act as catalysts to chemically alter the synthesis intermediates in a manner to promote formation of polymer sequences. Such photosensitive compounds include what are generally referred to as radiation-activated catalysts (RACs), and more specifically photo activated catalysts (PACs). The RACs can by themselves chemically alter the synthesis intermediate or they can activate an autocatalytic compound which chemically alters the synthesis intermediate in a manner to allow the synthesis intermediate to chemically combine with a later added synthesis intermediate or other compound.

Arrays can also be synthesized in a combinatorial fashion by delivering monomers to cells of a support by mechanically constrained flowpaths. See Winkler et al., EP 624,059. Arrays can also be synthesized by spotting monomers reagents on to a support using an ink jet printer. See id. and Pease et al., EP 728,520.

cDNA probes can be prepared according to methods known in the art and further described herein, e.g., reverse-transcription PCR (RT-PCR) of RNA using sequence specific primers. Oligonucleotide probes can be synthesized chemically. Sequences of the genes or cDNA from which probes are made can be obtained, e.g., from GenBank, other public databases or publications.

Nucleic acid probes can be natural nucleic acids, chemically modified nucleic acids, e.g., composed of nucleotide analogs, as long as they have activated hydroxyl groups compatible with the linking chemistry. The protective groups can, themselves, be photolabile. Alternatively, the protective groups can be labile under certain chemical conditions, e.g., acid. In this example, the surface of the solid support can contain a composition that generates acids upon exposure to light. Thus, exposure of a region of the substrate to light generates acids in that region that remove the protective groups in the exposed region. Also, the synthesis method can use 3′- protected 5′-0-phosphoramidite-activated deoxynucleoside. In this case, the oligonucleotide is synthesized in the 5′ to 3′ direction, which results in a free 5′ end.

Oligonucleotides of an array can be synthesized using a 96 well automated multiplex oligonucleotide synthesizer (A.M.O.S.) that is capable of making thousands of oligonucleotides (Lashkari et al., PNAS 93: 7912, 1995).

It will be appreciated that oligonucleotide design is influenced by the intended application. For example, it may be desirable to have similar melting temperatures for all of the probes. Accordingly, the length of the probes are adjusted so that the melting temperatures for all of the probes on the array are closely similar (it will be appreciated that different lengths for different probes may be needed to achieve a particular T[m] where different probes have different GC contents). Although melting temperature is a primary consideration in probe design, other factors are optionally used to further adjust probe construction, such as selecting against primer self-complementarity and the like.

Arrays, e.g., microarrrays, may conveniently be stored following fabrication or purchase for use at a later time. Under appropriate conditions, the subject arrays are capable of being stored for at least about 6 months and may be stored for up to one year or longer. Arrays are generally stored at temperatures between about −20° C., to room temperature, where the arrays are preferably sealed in a plastic container, e.g. bag, and shielded from light.

5.3 Hybridizing the Target Nucleic Acid to the Microarray

The next step is to contact the target nucleic acids with the array under conditions sufficient for binding between the target nucleic acids and the probes of the array. In a preferred embodiment, the target nucleic acids will be contacted with the array under conditions sufficient for hybridization to occur between the target nucleic acids and probes on the microarray, where the hybridization conditions will be selected in order to provide for the desired level of hybridization specificity.

Contact of the array and target nucleic acids involves contacting the array with an aqueous medium comprising the target nucleic acids. Contact may be achieved in a variety of different ways depending on specific configuration of the array. For example, where the array simply comprises the pattern of size separated probes on the surface of a “plate-like” rigid substrate, contact may be accomplished by simply placing the array in a container comprising the target nucleic acid solution, such as a polyethylene bag, and the like. In other embodiments where the array is entrapped in a separation media bounded by two rigid plates, the opportunity exists to deliver the target nucleic acids via electrophoretic means. Alternatively, where the array is incorporated into a biochip device having fluid entry and exit ports, the target nucleic acid solution can be introduced into the chamber in which the pattern of target molecules is presented through the entry port, where fluid introduction could be performed manually or with an automated device. In multiwell embodiments, the target nucleic acid solution will be introduced in the reaction chamber comprising the array, either manually, e.g. with a pipette, or with an automated fluid handling device.

Contact of the target nucleic acid solution and the probes will be maintained for a sufficient period of time for binding between the target and the probe to occur. Although dependent on the nature of the probe and target, contact will generally be maintained for a period of time ranging from about 10 min to 24 hrs, usually from about 30 min to 12 hrs and more usually from about 1 hr to 6 hrs.

When using commercially available microarrays, adequate hybridization conditions are provided by the manufacturer. When using non-commercial microarrays, adequate hybridization conditions can be determined based on the following hybridization guidelines, as well as on the hybridization conditions described in the numerous published articles on the use of microarrays.

Nucleic acid hybridization and wash conditions are optimally chosen so that the probe “specifically binds” or “specifically hybridizes” to a specific array site, i.e., the probe hybridizes, duplexes or binds to a sequence array site with a complementary nucleic acid sequence but does not hybridize to a site with a non-complementary nucleic acid sequence. As used herein, one polynucleotide sequence is considered complementary to another when, if the shorter of the polynucleotides is less than or equal to 25 bases, there are no mismatches using standard base-pairing rules or, if the shorter of the polynucleotides is longer than 25 bases, there is no more than a 5% mismatch. Preferably, the polynucleotides are perfectly complementary (no mismatches). It can easily be demonstrated that specific hybridization conditions result in specific hybridization by carrying out a hybridization assay including negative controls.

Hybridization is carried out in conditions permitting essentially specific hybridization. The length of the probe and GC content will determine the Tm of the hybrid, and thus the hybridization conditions necessary for obtaining specific hybridization of the probe to the template nucleic acid. These factors are well known to a person of skill in the art, and can also be tested in assays. An extensive guide to the hybridization of nucleic acids is found in Tijssen (1993), “Laboratory Techniques in biochemistry and molecular biology-hybridization with nucleic acid probes.” Generally, stringent conditions are selected to be about 5° C., lower than the thermal melting point (Tm) for the specific sequence at a defined ionic strength and pH. The Tm is the temperature (under defined ionic strength and pH) at which 50% of the target sequence hybridizes to a perfectly matched probe. Highly stringent conditions are selected to be equal to the Tm point for a particular probe. Sometimes the term “Td” is used to define the temperature at which at least half of the probe dissociates from a perfectly matched target nucleic acid. In any case, a variety of estimation techniques for estimating the Tm or Td are available, and generally described in Tijssen, supra. Typically, G-C base pairs in a duplex are estimated to contribute about 3° C., to the Tm, while A-T base pairs are estimated to contribute about 2° C., up to a theoretical maximum of about 80-100° C. However, more sophisticated models of Tm and Td are available and appropriate in which G-C stacking interactions, solvent effects, the desired assay temperature and the like are taken into account. For example, probes can be designed to have a dissociation temperature (Td) of approximately 60° C., using the formula: Td =(((((3×#GC)+(2×#AT))×37)−562)/#bp)−5; where #GC, #AT, and #bp are the number of guanine-cytosine base pairs, the number of adenine-thymine base pairs, and the number of total base pairs, respectively, involved in the annealing of the probe to the template DNA.

The stability difference between a perfectly matched duplex and a mismatched duplex, particularly if the mismatch is only a single base, can be quite small, corresponding to a difference in Tm between the two of as little as 0.5 degrees (See Tibanyenda, N. et al., Eur. J. Biochem. 139:19, 1984 and Ebel, S. et al., Biochem. 31:12083, 1992). More importantly, it is understood that as the length of the homology region increases, the effect of a single base mismatch on overall duplex stability decreases.

Theory and practice of nucleic acid hybridization is described, e.g., in S. Agrawal (ed.) Methods in Molecular Biology, volume 20; and Tijssen (1993) “Laboratory Techniques in biochemistry and molecular biology-hybridization with nucleic acid probes”, e.g., part I chapter 2 “Overview of principles of hybridization and the strategy of nucleic acid probe assays”, Elsevier, N.Y., provide a basic guide to nucleic acid hybridization.

Certain microarrays are of “active” nature, i.e., they provide independent electronic control over all aspects of the hybridization reaction (or any other affinity reaction) occurring at each specific microlocation. These devices provide a new mechanism for affecting hybridization reactions which is called electronic stringency control (ESC). Such active devices can electronically produce “different stringency conditions” at each microlocation. Thus, all hybridizations can be carried out optimally in the same bulk solution. These arrays are described in Sosnowski et al., U.S. Pat. No. 6,051,380.

In a preferred embodiment, background signal is reduced by the use of a detergent (e.g., C-TAB) or a blocking reagent (e.g., sperm DNA, cot-1 DNA, etc.) during the hybridization to reduce non-specific binding. In a particularly preferred (embodiment, the hybridization is performed in the presence of about 0.5 mg/ml DNA (e.g., herring sperm DNA). The use of blocking agents in hybridization is well known to those of skill in the art (see, e.g., Chapter 8 in Laboratory Techniques in Biochemistry and Molecular Biology, Vol. 24: Hybridization With Nucleic Acid Probes, P. Tijssen, ed. Elsevier, N.Y., (1993)).

The method may or may not further comprise a non-bound label removal step prior to the detection step, depending on the particular label employed on the target nucleic acid. For example, in certain assay formats (e.g., “homogenous assay formats”) a detectable signal is only generated upon specific binding of target to probe. As such, in these assay formats, the hybridization pattern may be detected without a non-bound label removal step. In other embodiments, the label employed will generate a signal whether or not the target is specifically bound to its probe. In such embodiments, the non-bound labeled target is removed from the support surface. One means of removing the non-bound labeled target is to perform the well known technique of washing, where a variety of wash solutions and protocols for their use in removing non-bound label are known to those of skill in the art and may be used. Alternatively, non-bound labeled target can be removed by electrophoretic means.

Where all of the target sequences are detected using the same label, different arrays will be employed for each physiological source (where different could include using the same array at different times). The above methods can be varied to provide for multiplex analysis, by employing different and distinguishable labels for the different target populations (representing each of the different physiological sources being assayed). According to this multiplex method, the same array is used at the same time for each of the different target populations.

In another embodiment, hybridization is monitored in real time using a charge-coupled device (CCD) imaging camera (Guschin et al., Anal. Biochem. 250:203, 1997). Synthesis of arrays on optical fibre bundles allows easy and sensitive reading (Healy et al., Anal. Biochem. 251:270, 1997). In another embodiment, real time hybridization detection is carried out on microarrays without washing using evanescent wave effect that excites only fluorophores that are bound to the surface (see, e.g., Stimpson et al., PNAS 92:6379, 1995).

5.4 Detecting Hybridized Nucleic Acids and Analyzing the Results from the Microarray

The above steps result in the production of hybridization patterns of target nucleic acid on the array surface. These patterns may be visualized or detected in a variety of ways, with the particular manner of detection being chosen based on the particular label of the target nucleic acid. Representative detection means include scintillation counting, autoradiography, fluorescence measurement, colorimetric measurement, light emission measurement, light scattering, and the like.

One method of detection includes an array scanner that is commercially available from Affymetrix (Santa Clara, Calif.), e.g., the 417TM Arrayer, the 418TM Array Scanner, or the Agilent GeneArrayTM Scanner. This scanner is controlled from the system computer with a WindowsR interface and easy-to-use software tools. The output is a 16-bit.tif file that can be directly imported into or directly read by a variety of software applications. Preferred scanning devices are described in, e.g., U.S. Pat. Nos. 5,143,854 and 5,424,186.

When fluorescently labeled probes are used, the fluorescence emissions at each site of a transcript array can be detected by scanning confocal laser microscopy. In one embodiment, a separate scan, using the appropriate excitation line, is carried out for each of the two fluorophores used. Alternatively, a laser can be used that allows simultaneous specimen illumination at wavelengths specific to the two fluorophores and emissions from the two fluorophores can be analyzed simultaneously (see Shalon et al., Genome Research 6:639-645, 1996). In a preferred embodiment, the arrays are scanned with a laser fluorescent scanner with a computer controlled X-Y stage and a microscope objective. Sequential excitation of the two fluorophores can be achieved with a multi-line, mixed gas laser and the emitted light is split by wavelength and detected with two photomultiplier tubes. In one embodiment in which fluorescent target nucleic acids are used, the arrays may be scanned using lasers to excite fluorescently labeled targets that have hybridized to regions of probe arrays, which can then be imaged using charged coupled devices (“CCDs”) for a wide field scanning of the array. Fluorescence laser scanning devices are described, e.g., in Schena et al., supra. Alternatively, the fiber-optic bundle described by Ferguson et al., Nature Biotech. 14:1681-1684, 1996, may be used to monitor mRNA abundance levels.

Following the data gathering operation, the data will typically be reported to a data analysis operation. To facilitate the sample analysis operation, the data obtained by the reader from the device will typically be analyzed using a digital computer. Typically, the computer will be appropriately programmed for receipt and storage of the data from the device, as well as for analysis and reporting of the data gathered, e.g., subtraction of the background, deconvolution of multi-color images, flagging or removing artifacts, verifying that controls have performed properly, normalizing the signals, interpreting fluorescence data to determine the amount of hybridized target, normalization of background and single base mismatch hybridizations, and the like. In a preferred embodiment, a system comprises a search function that allows one to search for specific patterns, e.g., patterns relating to differential gene expression, e.g., between the expression profile of a sample from a patient with cognitive impairments and the expression profile of a counterpart normal subject. A system preferably allows one to search for patterns of gene expression between more than two samples.

A desirable system for analyzing data is a general and flexible system for the visualization, manipulation, and analysis of gene expression data. Such a system preferably includes a graphical user interface for browsing and navigating through the expression data, allowing a user to selectively view and highlight the genes of interest. The system also preferably includes sort and search functions and is preferably available for general users with PC, Mac or Unix workstations. Also preferably included in the system are clustering algorithms that are qualitatively more efficient than existing ones. The accuracy of such algorithms is preferably hierarchically adjustable so that the level of detail of clustering can be systematically refined as desired.

Various algorithms are available for analyzing the gene expression profile data, e.g., the type of comparisons to perform. In certain embodiments, it is desirable to group genes that are co-regulated. This allows the comparison of large numbers of profiles. A preferred embodiment for identifying such groups of genes involves clustering algorithms (for reviews of clustering algorithms, see, e.g., Fukunaga, 1990, Statistical Pattern Recognition, 2nd Ed., Academic Press, San Diego; Everitt, 1974, Cluster Analysis, London: Heinemann Educ. Books; Hartigan, 1975, Clustering Algorithms, New York: Wiley; Sneath and Sokal, 1973, Numerical Taxonomy, Freeman; Anderberg, 1973, Cluster Analysis for Applications, Academic Press: New York).

Clustering analysis is useful in helping to reduce complex patterns of thousands of time curves into a smaller set of representative clusters. Some systems allow the clustering and viewing of genes based on sequences. Other systems allow clustering based on other characteristics of the genes, e.g., their level of expression (see, e.g., U.S. Pat. No. 6,203,987). Other systems permit clustering of time curves (see, e.g. U.S. Pat. No. 6,263,287). Cluster analysis can be performed using the hclust routine (see, e.g., “hclust”routine from the software package S-Plus, MathSoft, Inc., Cambridge, Mass.).

In some specific embodiments, genes are grouped according to the degree of co-variation of their transcription, presumably co-regulation, as described in U.S. Pat. No. 6,203,987. Groups of genes that have co-varying transcripts are termed “genesets.” Cluster analysis or other statistical classification methods can be used to analyze the co-variation of transcription of genes in response to a variety of perturbations, e.g. caused by a disease or a drug. In one specific embodiment, clustering algorithms are applied to expression profiles to construct a “similarity tree” or “clustering tree” which relates genes by the amount of co-regulation exhibited. Genesets are defined on the branches of a clustering tree by cutting across the clustering tree at different levels in the branching hierarchy.

In some embodiments, a gene expression profile is converted to a projected gene expression profile. The projected gene expression profile is a collection of geneset expression values. The conversion is achieved, in some embodiments, by averaging the level of expression of the genes within each geneset. In some other embodiments, other linear projection processes may be used. The projection operation expresses the profile on a smaller and biologically more meaningful set of coordinates, reducing the effects of measurement errors by averaging them over each cellular constituent sets and aiding biological interpretation of the profile.

Values that can be compared include gross expression levels; averages of expression levels, e.g., from different experiments, different samples from the same subject or samples from different subjects; and ratios of expression levels.

5.5 Data Analysis Methods for the Microarray

Comparison of the expression levels of one or more genes which are up- or down-regulated in response to the muscle wasting with reference to expression levels in the absence of muscle wasting, e.g., expression levels characteristic of a disease or in normal subject, is preferably conducted using computer systems. In one embodiment, one or more expression levels are obtained from two samples and these two sets of expression levels are introduced into a computer system for comparison. In a preferred embodiment, one set of one or more expression levels is entered into a computer system for comparison with values that are already present in the computer system, or in computer-readable form that is then entered into the computer system.

In one embodiment, the invention provides a computer readable form of the gene expression profile data of the invention, or of values corresponding to the level of expression of at least one gene which is up-regulated in response to inhibition of cognitive impairment in a subject. The values can be mRNA expression levels obtained from experiments, e.g., microarray analysis. The values can also be mRNA levels normalized relative to a reference gene whose expression is constant in numerous cells under numerous conditions, e.g., GAPDH. In other embodiments, the values in the computer are ratios of, or differences between, normalized or non-normalized mRNA levels in different samples.

The computer readable medium may comprise values of at least 2, at least 3, at least 5, 10, 20, 50, 100, 200, 500 or more genes. In a preferred embodiment, the computer readable medium comprises at least one expression profile.

Gene expression data can be in the form of a table, such as an Excel table. The data can be alone, or it can be part of a larger database, e.g., comprising other expression profiles, e.g., publicly available database. The computer readable form can be in a computer. In another embodiment, the invention provides a computer displaying the gene expression profile data.

The invention provides methods in which the level of expression of a single gene can be compared in two or more cells or tissue samples. In some embodiments, the level of expression of a plurality of genes is compared. For example, the level of expression of at least 2, at least 3, at least 5, 10, 20, 50, 100, 200, 500 or more genes. In an embodiment, expression profiles are compared.

In one embodiment, the invention provides a method for determining the similarity between the level of expression of one or more genes which are up-regulated in response to inhibition of cognitive impairment. The method preferably comprises obtaining the level of expression of one or more genes which are up-regulated in response to inhibition of cognitive impairment in a first sample and entering these values into a computer comprising (i) a database including records comprising values corresponding to levels of expression of one or more genes in a control untreated sample, and (ii) processor instructions, e.g., a user interface, capable of receiving a selection of one or more values for comparison purposes with data that is stored in the computer. The computer may further comprise a means for converting the comparison data into a diagram or chart or other type of output.

In one embodiment, the invention provides a system that comprises a means for receiving gene expression data for one or a plurality of genes; a means for comparing the gene expression data from each of said one or plurality of genes to a common reference frame; and a means for presenting the results of the comparison. This system may further comprise a means for clustering the data.

In another embodiment, the invention provides a computer program for analyzing gene expression data comprising (i) a computer code that receives as input gene expression data for a plurality of genes and (ii) a computer code that compares said gene expression data from each of said plurality of genes to a common reference frame.

The invention also provides a machine-readable or computer-readable medium including program instructions for performing the following steps: (i) comparing a plurality of values corresponding to expression levels of one or more genes which are up-regulated in response to inhibition of NMD in a query cell with a database including records comprising reference expression of one or more reference cells and an annotation of the type of cell; and (ii) indicating to which cell the query cell is most similar based on similarities of expression levels.

The relative levels of expression, e.g., abundance of an mRNA, in two biological samples can be scored as a perturbation (relative abundance difference) or as not perturbed (i.e., the relative abundance is the same). For example, a perturbation can be a difference in expression levels between the two sources of RNA of at least a factor of about 25% (RNA from one source is 25% more abundant in one source than the other source), more usually about 50%, even more often by a factor of about 2 (twice as abundant), 3 (three times as abundant) or 5 (five times as abundant). Perturbations can be used by a computer for calculating and expressing comparisons.

Preferably, in addition to identifying a perturbation as positive or negative, it is advantageous to determine the magnitude of the perturbation. This can be carried out, as noted above, by calculating the ratio of the emission of the two fluorophores used for differential labeling, or by analogous methods that will be readily apparent to those of skill in the art.

The computer readable medium may further comprise a pointer to a descriptor of the level of expression or expression profile, e.g., from which source it was obtained, e.g., from which patient it was obtained. A descriptor can reflect the stage of disease, the therapy that the patient is undergoing or any other descriptions of the source of expression levels.

In operation, the means for receiving gene expression data, the means for comparing the gene expression data, the means for presenting, the means for normalizing, and the means for clustering within the context of the systems of the present invention can involve a programmed computer with the respective functionalities described herein, implemented in hardware or hardware and software; a logic circuit or other component of a programmed computer that performs the operations specifically identified herein, dictated by a computer program; or a computer memory encoded with executable instructions representing a computer program that can cause a computer to function in the particular fashion described herein. Those skilled in the art will understand that the systems and methods of the present invention may be applied to a variety of systems, including IBM-compatible personal computers running MS-DOS or Microsoft Windows. Additionally the personal computer would have all of the hardware and software components normally associated with such a system such that the user would have capable memory, network connectivity, printing capability and programming capability with various computer languages. With the proper computer system the user could first load expression profile data into the computer system, U.S. Pat. No. 6,203,987. Geneset profile definitions are loaded into the memory from the storage media or from a remote computer, preferably from a dynamic geneset database system, through the network. Next the user causes execution of projection software which performs the steps of converting expression profile to projected expression profiles. The projected expression profiles are then displayed.

All of the above-cited references and publications are hereby incorporated by reference.

### EXAMPLIFICATION

### Example I

### Multiple Types of Skeletal Muscle Atrophy Involve a Common Program of Changes in Gene Expression

Abstract Skeletal muscle atrophy is a debilitating response to starvation and many systemic diseases including diabetes, cancer and renal failure. We had proposed that a common set of transcriptional adaptations underlie the loss of muscle mass in these different states. To test this hypothesis, we have used cDNA microarrays to compare the changes in content of specific mRNAs in muscles atrophying from different causes. We compared muscles from fasted mice, from rats with cancer cachexia, streptozotocin-induced diabetes mellitus, and uremia induced by subtotal nephrectomy and from pair-fed control rats. Although the content of>90% of mRNAs did not change, including those for the myofibrillar apparatus, we found a common set of genes (termed atrogins) that were induced or suppressed in muscles in these four catabolic states. Among the strongly induced genes were many involved in protein degradation, including polyubiquitins, Ub fusion proteins, the Ub ligases atrogin-1/MAFbx and MuRF-1, multiple but not all subunits of the 20S proteasome and its 19S regulator and cathepsin L. Many genes required for ATP production and late steps in glycolysis were down-regulated, as were many transcripts for extracellular matrix proteins. Some genes not previously implicated in muscle atrophy were dramatically up-regulated (lipin, metallothionein, AMP deaminase, RNA helicase related protein, TG-interacting factor) and several growth-related mRNAs were down-regulated (P311, JUN, IGF-1-BP5). Thus, different types of muscle atrophy share a common transcriptional program that is activated in many systemic diseases.

A general loss of skeletal muscle mass is a characteristic, debilitating response to fasting, as well as many severe diseases including advanced cancer, renal failure, sepsis and diabetes (1). Atrophy of specific muscles results from their disuse or denervation. In most types of muscle atrophy overall rates of protein synthesis are suppressed and rates of protein degradation are consistently elevated; this response accounts for the majority of the rapid loss of muscle protein. In a variety of animal models of human diseases [e.g. fasting (2, 3), diabetes (4), cancer cachexia (5-7), acidosis (8), sepsis (9), disuse atrophy (10), denervation (2) and glucocorticoid treatment (11)], most of the accelerated proteolysis in muscle appears due to an activation of the Ub-proteasome pathway (12). For example, in these diverse conditions the muscles show a two-to-fourfold increase in levels of mitochondrial RNA for polyubiquitin and certain proteasome subunits. A similar induction of components of the Ub-proteasome pathway has also been found in atrophying human muscle (13, 14). Whereas weight loss in fasting and diabetes involve reduced levels of insulin and elevated glucocorticoid levels, tumor cachexia and sepsis are often associated with increased TNFα, and renal failure with metabolic acidosis. Despite the varied physiological or pathophysiological stimuli for muscle atrophy, earlier studies revealed striking similarities in the transcriptional adaptations of genes encoding certain components of the Ub-proteasome pathway. Therefore, we hypothesized that atrophying muscles exhibit a coordinated series of transcriptional adaptations that constitute a common atrophy program (15, 16).

To test whether a common program of transcriptional adaptations indeed occur in muscle as it undergoes atrophy and to better understand these catabolic states, we used cDNA microarrays to compare mRNA content of normal muscle with atrophying ones from fasted mice and rats with renal failure, cancer or diabetes. Transcriptional profiling using microarrays is ideal for defining the atrophy-induced changes in mRNA content, with the understanding that these changes may reflect alteration in mRNA stability as well as gene transcription. Many authors have used this or other genomic techniques to study transcriptional changes in muscle in certain conditions (17-22), although no comparisons of such profiles from different types of muscle atrophy has been performed; the common set of genes induced and suppressed during atrophy has therefore not been investigated.

As an initial step, we studied the transcriptional changes in muscles of fasted mice (20). Besides confirming the increases in mRNA levels shown by Northern blot for polyUb and certain 20S proteasome subunits, the study demonstrated differential expression of other genes with diverse functions (20). During these studies we identified and cloned a previously unknown muscle-specific Ub ligase that is dramatically induced in muscle wasting not only in fasting, but also in tumor-induced cachexia, diabetes, chronic renal failure and dexamethasone treatment (16). Simultaneously Bodine et al. showed this same gene is also induced in atrophy by denervation or disuse (19). We named this gene atrogin-1, as it was the first new gene identified in our attempts to define the atrophy program. Another muscle specific Ub-ligase, MuRF-1, was shown to be markedly induced upon denervation and disuse (19). The present study tested whether its level also rises in atrophying muscles due to fasting or catabolic diseases.

In fasting, protein breakdown in muscle provides the organism with a source of amino acids for gluconeogenesis. We have now used the microarray approach (20) to test whether the response to fasting involve similar adaptations to those in muscles atrophying due to cancer cachexia, uncontrolled diabetes, and chronic renal failure. In each of these animal models, atrophy, especially of fast-twitch muscle fibers, has been extensively documented. Overall rates of protein breakdown, as measured in isolated muscles in vitro are increased by 40-65% (Table 1). mRNA for some components of the Ub-proteasome pathway are elevated by two-to-threefold (2-7, 23) and rates of Ub conjugation in cell-free extracts are enhanced (24, 25). In fasting, some changes found in muscle might be specifically related to the inadequate caloric or nutrient intake. Therefore, it was important to compare changes in mRNA in muscles after tumor implantation and renal failure with muscles from pair-fed animals, since anorexia often accompanies the metabolic disturbances in these conditions. This approach allowed us to identify the transcriptional changes resulting directly from the disease process and to avoid the potential complications of nutrient deprivation.

Defining a common transcriptional profile in a range of wasting diseases should increase our understanding of the critical adaptations associated with muscle atrophy independent of the cause of the muscle wasting. Beyond helping us understand these important responses, this analysis also may identify therapeutic targets for retarding the atrophy process (26). Furthermore, this study lays a basis for comparison with the transcriptional changes that occur in specific muscles in denervation or disuse atrophy. We show here that many genes, which we term atrogins, are differentially expressed in multiple types of atrophy and comprise a common atrophy program.

Materials and Methods

Transcriptional profiling was performed on gastrocnemius muscles from mice or rats with muscle wasting induced by fasting (20), diabetes mellitus (4, 27), renal failure (23), and tumor implantation (5). All animal experiments were approved by institutional review boards.

Microarray Hybridization and Data Analysis

RNA extraction from muscles and performance of micoarray hybridizations by Incyte Inc. (St. Louis, Mo., USA) were as described previously (20). Analysis was performed using Rosetta Resolver (Rosetta Inpharmatics, Seattle, Wash., USA), Mocrosoft Excel and the relational database Microsoft Access. Raw data files from hybridizations that passed quality control tests applied by Incyte were loaded into Resolver and analyzed using a specific Rosetta error model generated for Incyte microarrays (28). Resolver allows the combining of repeated experiments to yield single fold-change and significance values for data points common to multiple microarrays.

General Design of Experiments

For each experiment (defined below) RNA was extracted from a fresh set of muscles from a minimum of two experimental and control animals. Three hybridization experiments were performed with muscles from fasted mice, two using muscles from diabetic rats, two using muscles from rats with renal failure, and two using muscles from tumor-bearing rats. Each experiment involved hybridizing atrophying and control samples to both human (Human UniGEM 1 or 2) and mouse (Mouse GEM 1) microarrays. We previously showed that hybridizing mouse RNA to human cDNA microarrays yields reliable and useful results. Since the cDNA sequences on the human and mouse arrays represent overlapping but different sets of genes, this strategy allowed us to extend the range of genes analyzed (20). The extensive sequence similarity between rodent and human genes permits such cross-species hybridization. In one experiment for each condition, the samples previously labeled with Cy5 were labelled with Cy3 (and vice versa) to compensate for any nonlinearity in the emission signal intensity response curve for each fluorophore. One of the diabetes experiments was hybridized to a mouse microarray and failed to give technically satisfactory results at Incyte; it was not included in our analysis.

Defining Atrophy-Specific Genes or Atrogins

Results for each array were combined in Resolver to yield a single average fold change (atrophy/control) for each gene in each type of atrophy. Heatmaps shown in the figures were generated using the natural logarithm of this ratio using Heatmap Viewer 1.0 (Chang Bioscience, San Francisco, Calif., USA). Increased expression is indicated by an increasing intensity of red and decreased expression by an increasing intensity of green. When results within each atrophy state were combined, Resolver calculated the probability of differential expression for each gene. We define those genes with an average P value of<0.05 in all four types of muscle atrophy as atrogins and other genes as either shared (p<0.05 in two or three states only), disease-specific (p<0.005 in one state but p>0.2 in the other three conditions), or unchanged (p>0.05). Cut-offs adopted for the disease-specific genes are arbitrary but were designed to minimize the false-positive rate (0.5%) for the catabolic state in question while also controlling the false-negative rate in the three other catabolic states (i.e. to minimize the probability that a gene identified as differentially expressed in only one catabolic state was in fact differentially expressed in other states) (Table 2).

Analysis of Transcription Factor Binding Motifs in the Upstream Regions of Atrogins

Rates of occurrence of the 124 transcription factor binding motifs in the TRANSFAC database were obtained in 3 kB of the 5′ region of 31 upregulated atrogins (Unigene clusters Mm.3238, Hs.61661, Hs.173685, Mm.2159, Mm.930, Hs.87417, Hs.194669, Hs.71819, Hs.25732, Mm.29891, Mm.14638, Hs.7879, Hs.112396, Mm.28548, Mm.28357, Mm.22749, Mm.25311, Mm.41792, Mm.6720, Mm.180499, Mm.28571, Hs.78466, Mm.30097, Mm.21874, Hs.182979, Hs.8765, Hs.28491, Hs.94360, Hs.5308, Hs.183842, Hs.18370) and 17 downregulated atrogins (Unigene clusters Hs.177584, Hs.172928, Hs.80691, Hs.115285, Hs.750, Hs.287820, Mm.578, Hs.17109, Hs.198951, Hs.2795, Mm.3156, Mm.2060, Mm.30000, Mm.4919, Hs.181013, Mm.147387, Mm.28683). Frequency values (per 1000 bp) were subsequently divided by the frequency of random occurrence of the motifs, calculated by the MatInspector program. For each motif, the occurrence frequency in up-regulated divided by down-regulated atrogins was calculated. Motif frequency was also measured in the 5′ region of 15 genes not differentially expressed in any of the atrophying muscles (Unigene clusters Hs.118442, Hs.197540, Hs.25450, Hs.26045, Hs.302131, Hs.348412, Hs.37616, Hs.75219, Mm. 16373, Mm.1764, Mm.179747, Mm.83615, Mm.2661).

### RESULTS

We performed cDNA microarray hybridizations on RNA derived from four models of human disease characterized by cachexia and marked muscle atrophy: fasting in mice, implantation of Yoshida hepatoma in rats, ⅞ nephrectomy resulting in uremia and acidosis in rats, and streptozotocin administration leading to uncontrolled diabetes mellitus in rats. At the time of analysis muscles In each group showed significant weight loss (13-29%), and overall rate of protein degradation was 40-63% faster than in muscles of control animals (Table 1). Thus, these muscles were undergoing rapid atrophy. Since the microarrays from the ⅞-nephrectomized, streptozotocin-treated and tumor-bearing animals were compared with ones from pair-fed control animals studied in parallel, changes in mRNA specifically reflect effects of the disease process, and not any associated decrease in food intake. The large number of fasting-specific transcriptional changes argues that our attempts to control for decreases in food intake in the disease models by pair-feeding regimes were largely successful and a unique pattern of transcription in fasting alone was still evident.

The results from hybridizations of muscle RNA to both mouse and human microarrays together yielded 16,392 individual gene sequences that could be analyzed in all four catabolic states (Table 2). Of these, 133 mRNAs (0.8%) were differentially expressed (i.e. p<0.05) in all four and were designated as atrogins. This number actually comprises 120 unique genes since some sequences were duplicated on the mouse and human arrays. Expression data for all genes defined as atrogins are included in Supplementary Table 1. Data for the disease-specific genes, the individual microarray outputs, and Supplementary Tables 1 and 2 can be found at http://agoldberg.med.harvard.edu/muscledatabase.

Protein Degradation

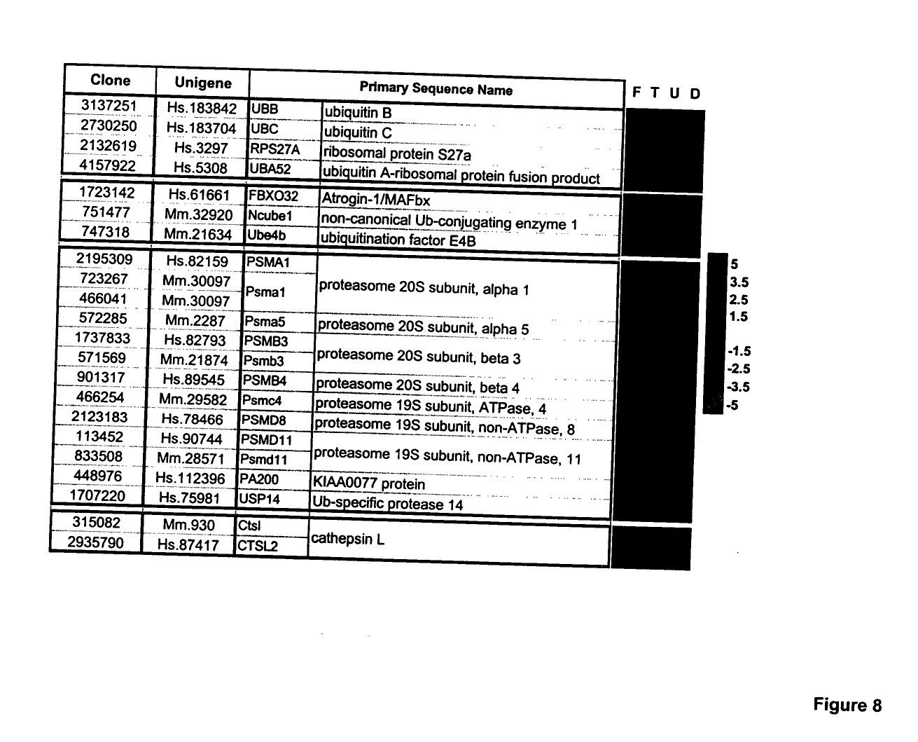

mRNAs for many genes involved in protein degradation were up-regulated in all four types of atrophying muscle (FIG. 8). As expected, nearly all these genes encoded components of the Ub-proteasome pathway, including the two polyUb genes, four different subunits of the 20S proteasome and three of the 19S proteasome regulator, some of which have been demonstrated by Northern blot analysis previously (20) (29). An additional four proteasome subunits and one 19S subunit were up-regulated in three of four atrophy states, and others in one or two catabolic states (see Supplementary Table 2 at http://agoldberg.med.harvard.edu/muscledatabase). Surprisingly five of the thirty-four 26S subunits were not differentially expressed in any of the atrophy conditions, and levels of none was repressed (see Supplementary Table 2). Of note was the˜threefold increase in mRNAs for PA200, a recently described component of nuclear proteasomes that activate peptide hydrolysis and has been proposed to play a role in DNA repair (30). Finally, in all types of atrophy, there was an induction of USP14, an isopeptidase that associates reversibly with the 19S complex (31), and may be important in recycling polyUb chains back to Ub monomers.

A marked induction mRNA levels for two Ub-extension proteins that are Carboxyl-terminal fusions between Ub and ribosomal proteins (RPS27A or UBA52) was consistently found in all types of atrophying muscle studied. These fusion proteins probably serve as a source of free Ub, in addition to the polyub genes, UBB and UBC, when proteolysis increases.

Ub Ligases

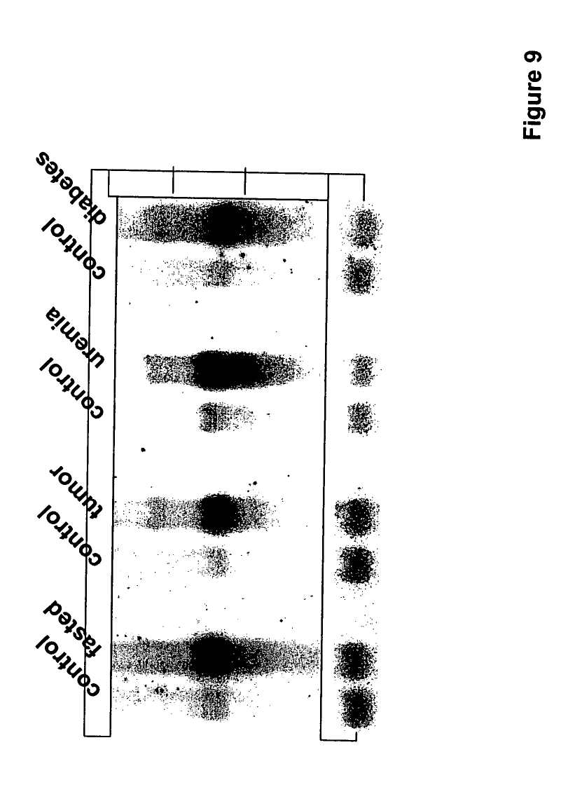

The Ub-ligase (E3), atrogin-1/MAFbx, was originally cloned because its mRNA was the most highly induced in muscle during fasting and its expression increases between 4 and 15-fold in different catabolic states (16). Atrogin is also strongly induced in muscles from septic rats (32), and after denervation or disuse (19). Furthermore, mice lacking this gene show reduced rates of disuse atrophy (19). Expression of another muscle-specific E3, MuRF-1, increases markedly after denervation or disuse (19), but was not present on our microarrays. To test whether it is of general importance in muscle atrophy, the same RNA was analyzed by Northern blot. Indeed, MuRF-1 mRNA was strongly induced in all four catabolic states (FIG. 9).

The dramatic induction of atrogin-1 and MuRF-1 contrasts with the lack of change in expression of many other Ub-conjugating enzymes (see Supplementary Table 2) including E1, many E2s, and the E3s, Nedd4, and E6AP. It is noteworthy that no significant change occurred in Ubr1(E3a) or E214k (components of the N-end rule pathway) whose mRNAs were found to rise by Northern analysis in muscles in diabetes (25), fasting (33, 34) and sepsis (35). Small increases (1.3 to 3-fold) were observed in mRNAs for the ubiquitination factor, E4B, which may act in combination with an E3 to increase the efficiency of Ub conjugation to proteins (36). Also increased in all catabolic states was mRNA for another Ub-carrier protein (E2), the noncanonical Ub-conjugating enzyme 1, whose role in muscle is unclear. Its yeast homologue, Ubc6, is involved in ubiquitinating proteins retro-translocated from the ER (37).

An important lysosomal cysteine protease, cathepsin L, was induced two-to threefold in all four catabolic states whereas mRNAs for the other lysosomal hydrolases was not (see Supplementary Table 2). Although lysosomes have only a limited role in the bulk of intracellular proteolysis, large increases in cathepsin L mRNA occur in muscle from septic, tumor-bearing, dexamethasone treated, and fasted rodents (18, 20). Some reports have suggested that cathepsin L may be found extracellularly (38); if so, this protease might play a special role in turnover of extracellular components during atrophy. No change in mRNA levels for a range of matrix metalloproteases (MMPs) was found. MMP-2 and MMP-9 are expressed in muscle (39), and have been proposed to function in remodeling of the extracellular matrix after disuse or denervation (40, 41). However, MMPs are induced late in disuse atrophy (40) and so may not be a feature of the rapid atrophy studied here.

ATP production and substrate metabolism. Genes encoding some key proteins in mitochondrial energy production, glucose and ketone body metabolism were differentially expressed in all four catabolic states (FIG. 10). mRNAs for seven different inner mitochondrial membrane proteins as well as mitochondrial creatine kinase, all of which participate in electron transport and/or ATP synthesis was reduced in all catabolic states examined in this study. mRNAs encoding the γ subunit of the glycogen phosphorylase kinase complex and several enzymes catalyzing later steps in glycolysis were reduced, as well as two components of the pyruvate dehydrogenase complex that regulate whether pyruvate is oxidized by the TCA cycle. Also reduced was expression of malate dehydrogenase, a key component of this cycle, as well as the malate-aspartate shuttle that brings reducing equivalents produced in glycolysis to the mitochondrion. Finally, mRNA for 3-oxo CoA transferase, required for oxidation of ketone bodies, was reduced in all four catabolic states. These changes in gene expression would be expected to suppress muscle's capacity to utilize glucose and reduce muscle energy production generally. Reduced glucose utilization is consistent with the lack of insulin in fasting and insulin resistance in cancer and renal failure; regulation of these steps has not been reported previously. These changes do not appear to support the idea that cachexia is a purely hypermetabolic response where substrates are utilized at accelerated rates (42). In our entire analysis, the inducible form of 6-phosphofructo-2-kinase (iPFK), which is also induced in some tumors (43), was the only atrogin not similarly regulated in all four catabolic states.

Kahn and coworkers recently described the transcriptional profile in muscle from mice made diabetic by prolonged treatment with streptozotocin (21), and observed coordinate suppression a different set of genes involved in glucose utilization. These workers also did not observe changes in many of the genes identified here as atrogins (e.g. Ub, proteasome subunits, metallothionein). However, the muscles studied by Yechoor et al. were from mice (instead of rats) and were treated longer with streptozotocin to create a more chronic, adapted diabetic state. Unfortunately, no analysis of the extent of muscle weight loss or rates of protein degradation was performed in that study, thus muscle wasting may have ceased at the time of their analysis.

Nitrogen metabolism Expression of three genes encoding enzymes for purine or polyamine catabolism was consistently increased, including 1) spermidine N1-acetyltansferase, a key enzyme in polyamine catabolism, which was induced about fivefold in muscles from uremic animals as well as 2) IMP dehydrogenase and 3) AMP deaminase 3, which are involved in the purine-nucleotide cycle. This cycle may play a key role in energy production in muscle (44) and is a source of ammonia derived from amino acid degradation. In muscle, ammonia is used to form glutamine from glutamate in a reaction catalyzed by glutamine synthase. Indeed, glutamine synthase is markedly increased in muscles from all four states. Glutamine production and export by muscle occur in a variety of catabolic conditions, including sepsis and trauma; simultaneously there is increased uptake of glutamine by the liver, lymphocytes and the kidney (45). This inter-organ flux of glutamine appears to help provide substrates for increased gluconeogensis and urinary ammonia generation in acidosis. No significant changes were noted in mRNAs for enzymes of branched chain amino acid metabolism, which are induced in fasting. However, transcript levels for an amino acid transporter, Slc7a8, did increase. Slc7a8 may be involved in exporting amino acids like glutamine, whose release from muscle increases when there is net proteolysis (46, 47).

Transcription Factors

No evidence was obtained for the simplistic view that atrophy involves a general repression of muscle gene expression (FIG. 11a), though three transcriptional activators associated with rapid growth—JUNB, MAF, and the mouse ortholog of human Snf2-related CBP activator protein (SRCAP)—had reduced mRNA levels in all the atrophy states studied and may reflect suppression of growth in wasting muscle. mRNA levels for the transcriptional repressor EZH1, which may stabilize heterochromatin, were increased. These changes would appear to favor a reduction in the transcription of a subset of genes in atrophy. On the other hand, mRNA for MAX, a MYC-related transcriptional activator, increased somewhat in all conditions studied. Foxo1, a member of the forkhead family of transcriptional activators, was strongly induced. Foxo1 has recently been implicated in the development of insulin resistance in type II diabetes in liver, pancreas, and adipose tissue (48). Perhaps the induction of Foxo1 in atrophying muscle may help to explain the insulin resistance in these catabolic states. mRNA for p23 telomerase binding protein, Tebp, which binds to DNA and interferes with some hormone-dependent transcription, and Nfe2l2 which responds to oxidative stress, increased in all atrophy states (49). ATF4, which regulates amino acid metabolism and resistance to oxidative stress, was increased (50). Finally, mRNA for Tgif, a homeobox gene that may repress certain transcriptional activators (51), was markedly increased.

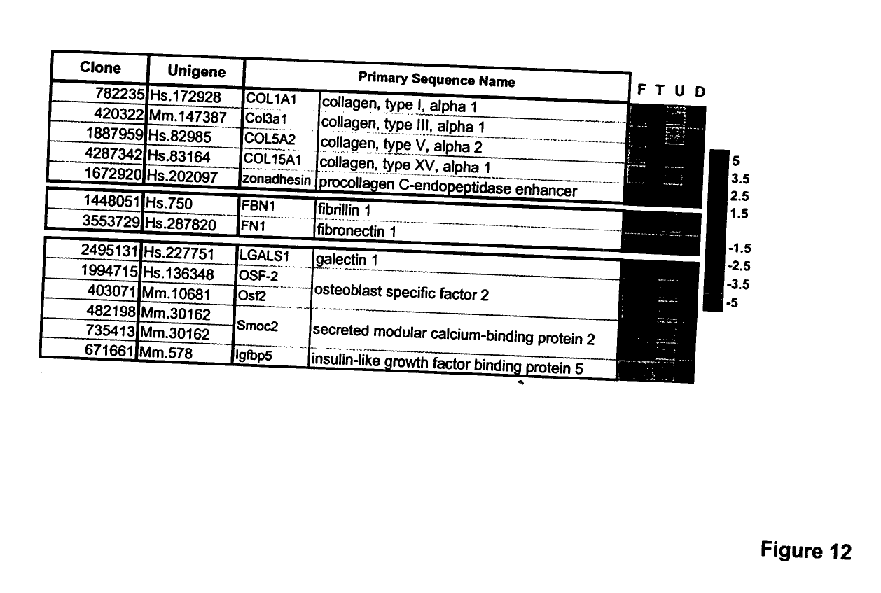

Extracellular matrix components. In the four catabolic states, mRNA levels were reduced for collagen I, III, V, and XV and the procollagen-C endopeptidase enhancer, which accelerates the maturational cleavage of the procollagen I C-propeptide in the extracellular compartment (FIG. 12). It was recently reported that mRNA for collagen III as well as levels of this protein decrease during disuse atrophy (17). mRNA levels for other matrix components, fibrillin and fibronectin, were reduced, as were those for OSF-2 a cell adhesion protein, and galectin 1, LGALS1, which has recently been shown to enhance myoblast fusion and induce myogenic differentiation of fibroblasts (52).

Genes involved in translational control. In fasting, a surprising increase was found in mRNAs for genes encoding certain translation initiation factors and the inhibitor of cap-dependent initiation, 4E binding protein (20). These adaptations could help reduce 5′ cap-dependent translation that occurs when growth factors and nutrients are decreased while allowing cap-independent translation of other key proteins that appear important in stressfull conditions (53). In all four conditions, mRNAs for translation initiation factors EIF4A2, EIF4G3, and EIF4EBP1, which act through cap-independent mechanisms increased (FIG. 11b). Despite the reduction in translation, transcript levels for a 60S ribosomal protein L12, and nucleolin, which are believed to participate in ribososmal assembly, increased in all four catabolic states. Finally, an RNA helicase-related protein containing a DEAD box motif was increased in all four states, with a particularly dramatic rise (up to 100-fold) in diabetes and renal failure. Although the precise role of this protein is unknown, similar RNA helicases are involved in ribosomal assembly, translation initiation, and RNA processing.

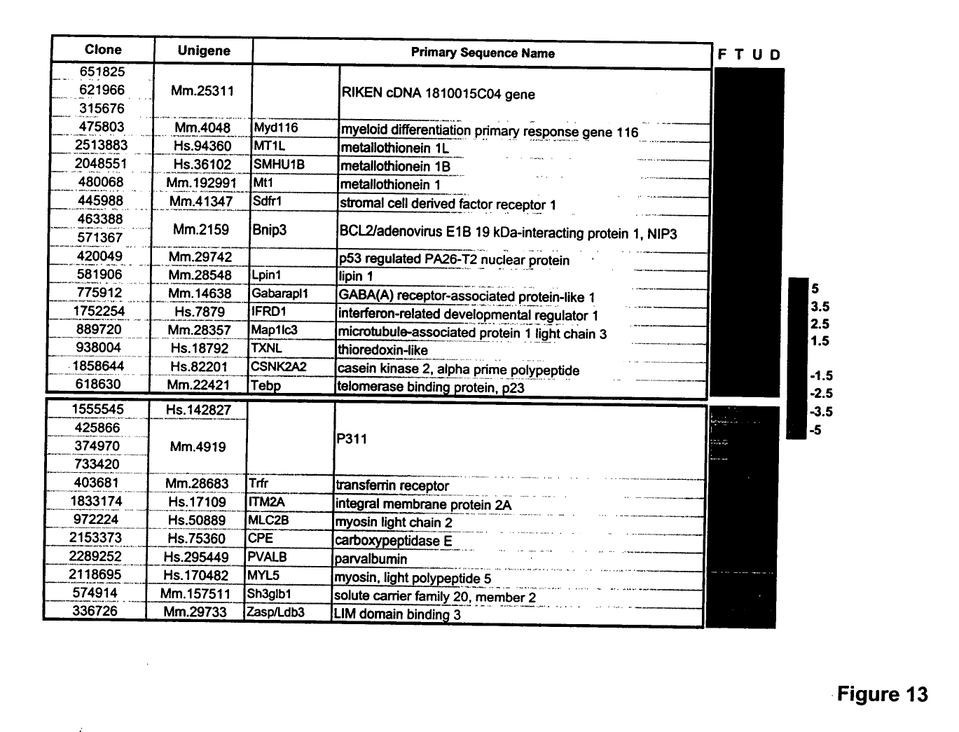

Metallothionein and other proteins induced in oxidative stress. Metallothionein was among the most strongly induced genes in all the atrophying muscles on both the human and mouse microarrays. Two of the 14 highly homologous human metallothionein genes present on the human microarrays, MT1B and MT1L, were dramatically induced (3- to 20-fold) (FIG. 13). Mouse Mt1 was induced 1.5- to 2.5-fold (FIG. 13); clones of Mt1 and Mt2, present only on the arrays from fasted mice, were induced 5-to 6-fold (see clones 1037652, 334351). Since the mouse Mt1 sequence is highly homologous to that of human MT1L and MT1B (data not shown), it is likely that rodent Mt1 transcripts from the atrophying muscles hybridized to the MT1L and MT1B cDNA fragments on the human arrays.

Metallothioneins are induced by heavy metals, glucocorticoids, and oxidative stress, and can protect cells against DNA damage from reactive oxygen species (54, 55). The mechanisms by which metallothionein exerts its protective effect are still unclear. A more modest increase in mRNA for the 32 kD thioredoxin-like-protein (56), which helps maintain the cytosol in a reduced state, was observed in all four atrophy states. As mentioned above, mRNA for Nfe2l2 and ATF4, transcription factors that regulate genes controlled by antioxidant response elements, increased in all four states (FIG. 11a). Thus, the atrophy program seems to include elements of a transcriptional response to oxidative stress.

Genes involved in muscle growth and differentiation. Insulin-like growth factor-1 is a major determinant of muscle growth and plays a key role in compensatory hypertrophy (57). It stimulates the PI-3-kinase and Akt signal transduction cascade whose activation can combat denervation and disuse atrophy (58). Although expression of IGF-1 or downstream signaling molecules did not change in the atrophying muscles, IGF-1 binding protein 5, which binds to the extracellular matrix and modulates the muscle's response to IGF-1 (59) was dramatically down-regulated in all atrophy conditions studied (FIG. 13).

The group of atrogins also included the proapoptotic gene, Bnip3, which was induced˜threefold in all four types of atrophy (FIG. 13). Bnip3 interacts with and can antagonize Bcl-2 (60); it is induced by hypoxia and acidosis, when it triggers cardiomyocyte cell death (61). However, there is no evidence for cell death in the reversible forms of muscle atrophy studied here.

mRNA levels decreased for two calcium binding proteins: parvalbumin, and secreted modular calcium binding protein 2 (Smoc2) (FIG. 13). Parvalbumin is found in fast-twitch fibers and binds cytosolic Ca²⁺, enabling rapid relaxation after contraction. Smoc2 is highly expressed by skeletal and vascular smooth muscle; although its role is unclear, up-regulation of Smoc2 occurs in vascular smooth muscle in response to stretch injury and is associated with smooth muscle proliferation (62).

Although large decreases in expression of myofibrillar proteins were anticipated, little or no evidence was obtained for suppression of the transcription of myofibrillar or cytoskeletal genes in atrophying muscle. Only two components of the myofibril—namely, two myosin light chain isoforms—feature in the group of down-regulated atrogins. (FIG. 12). Only mRNA for micotubule-associated protein Map1lc3 was strongly induced in all states whereas mRNA for LIM domain binding protein 3, which binds actinin and localizes to the Z-band of the myofibril (63), was suppressed (FIG. 12).

A large proportion of the atrogins were unknown genes or genes whose function in muscle is still obscure. Included in this group is the newly discovered gene lipin, which, when mutated, results in lipodystrophy (64). mRNA levels for lipin were markedly induced in all the types of atrophy (FIG. 13). Lipin may regulate lipid biosynthesis in the liver. It is phosphorylated by mTOR in response to insulin (65), and thus is controlled by the Akt signaling pathway. In addition, the interferon-related developmental regulator-1 (Ifrd1) was induced˜threefold in all 4 states (FIG. 13). Although Ifrd1 is necessary for normal muscle differentiation (66), its role in the development of atrophy is unknown. In contrast, mRNA for P311 was consistently reduced between 3- and 14-fold in these states (FIG. 13). p311 is an 8 kDa protein first found in neurons late in brain development and in invasive glioblastoma (67). P311 appears to be involved in smooth muscle differentiation in myofibroblasts by driving expression of actin and other muscle-specific genes (68).

The largest subgroup of atrogins was that for which no known function has been established. These included several genes with dramatic increases in mRNA levels of 7- to 8-fold (e.g., clones 619799, 651825, 621966) and others with even larger relative decreases in mRNA levels of up to 100-fold (clone 692699).

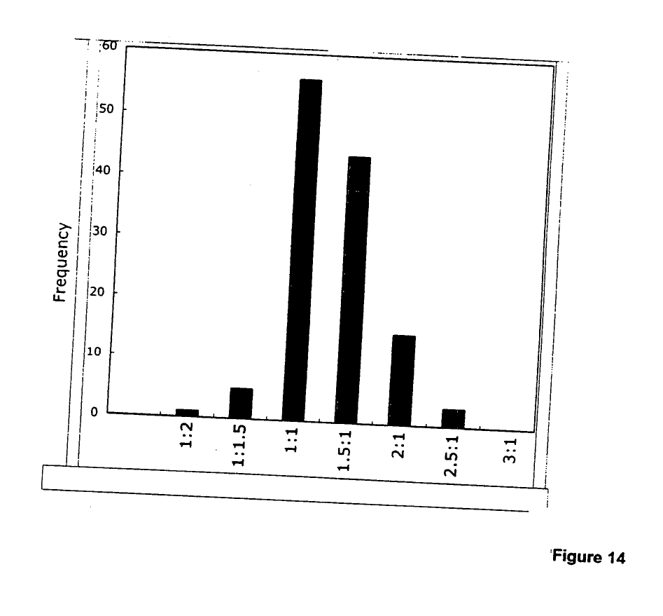

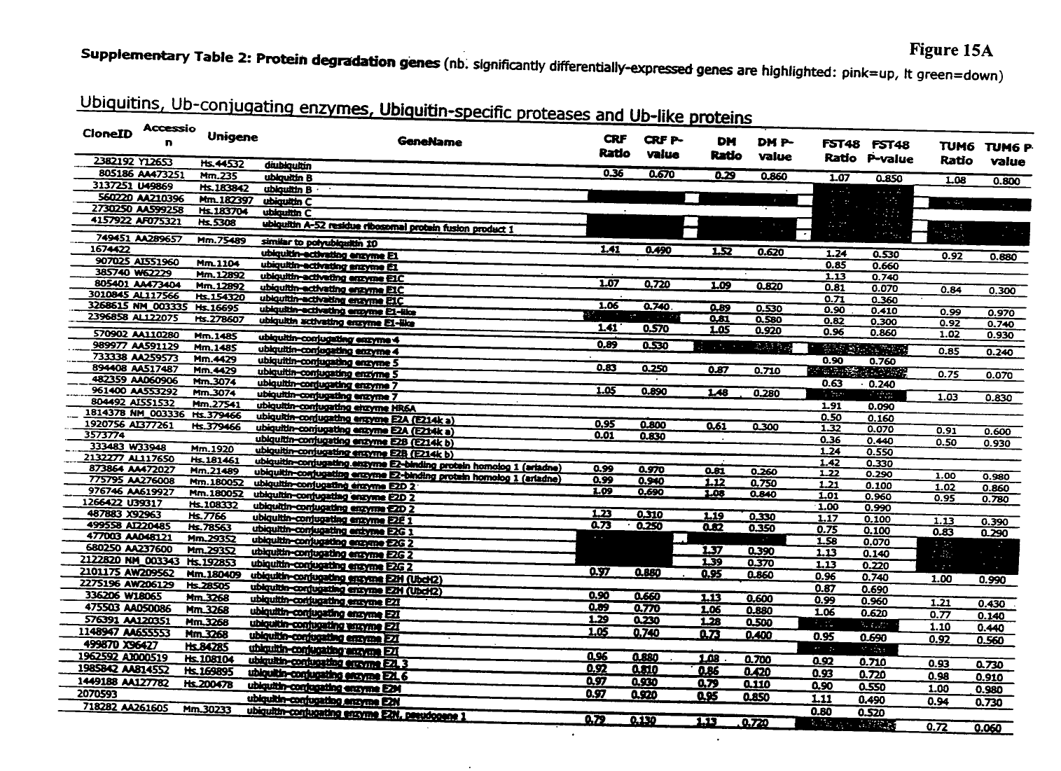

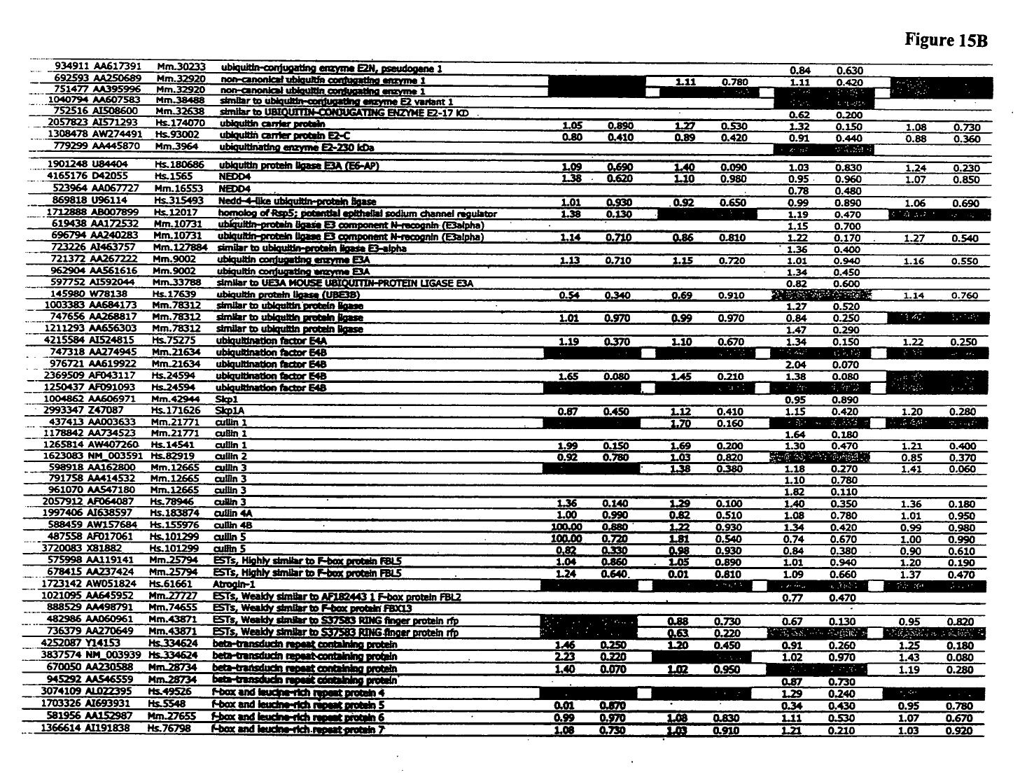

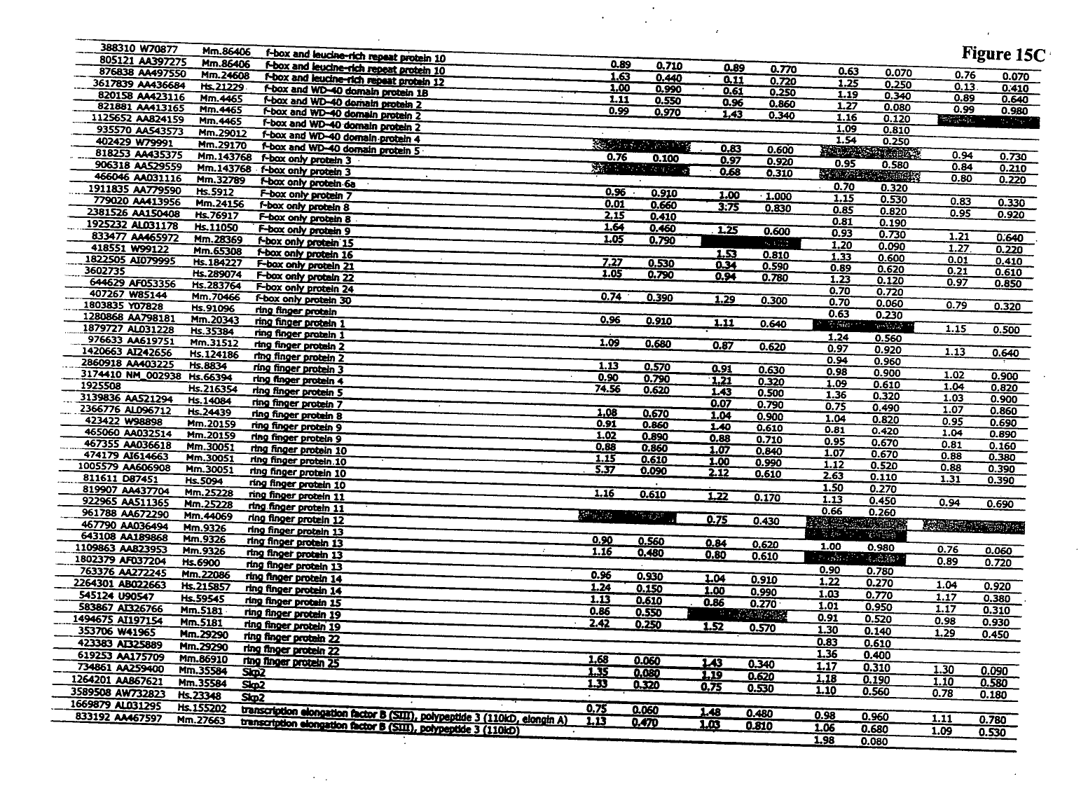

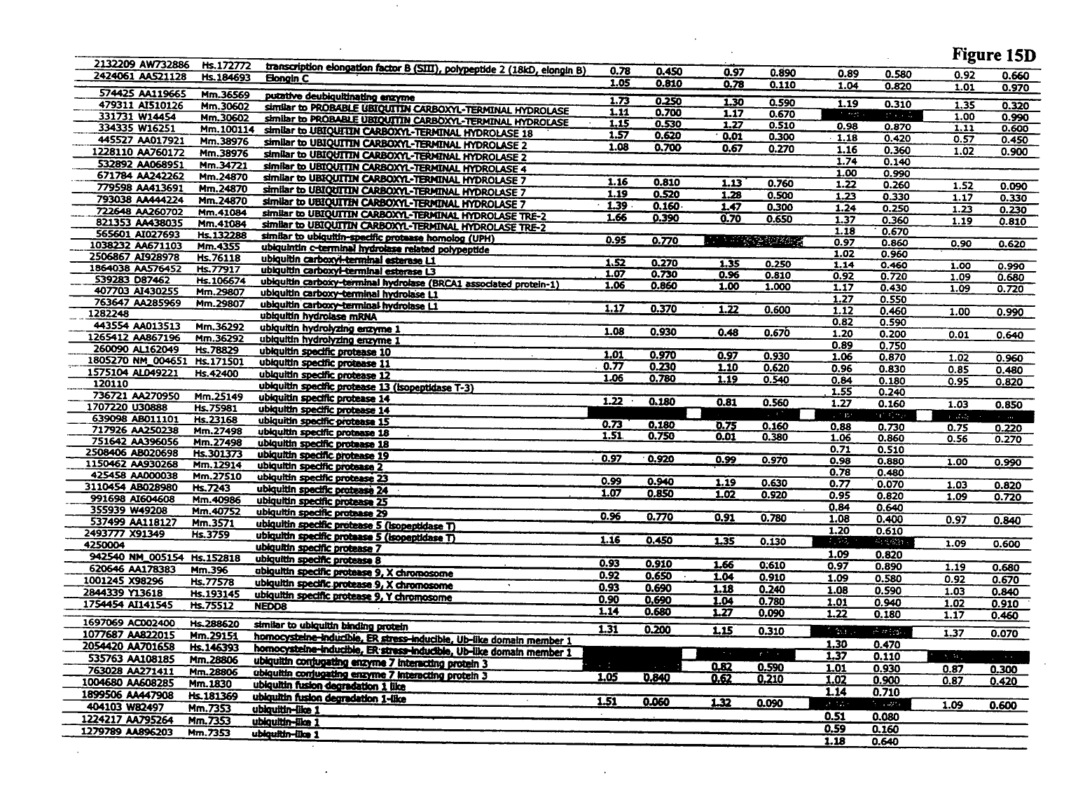

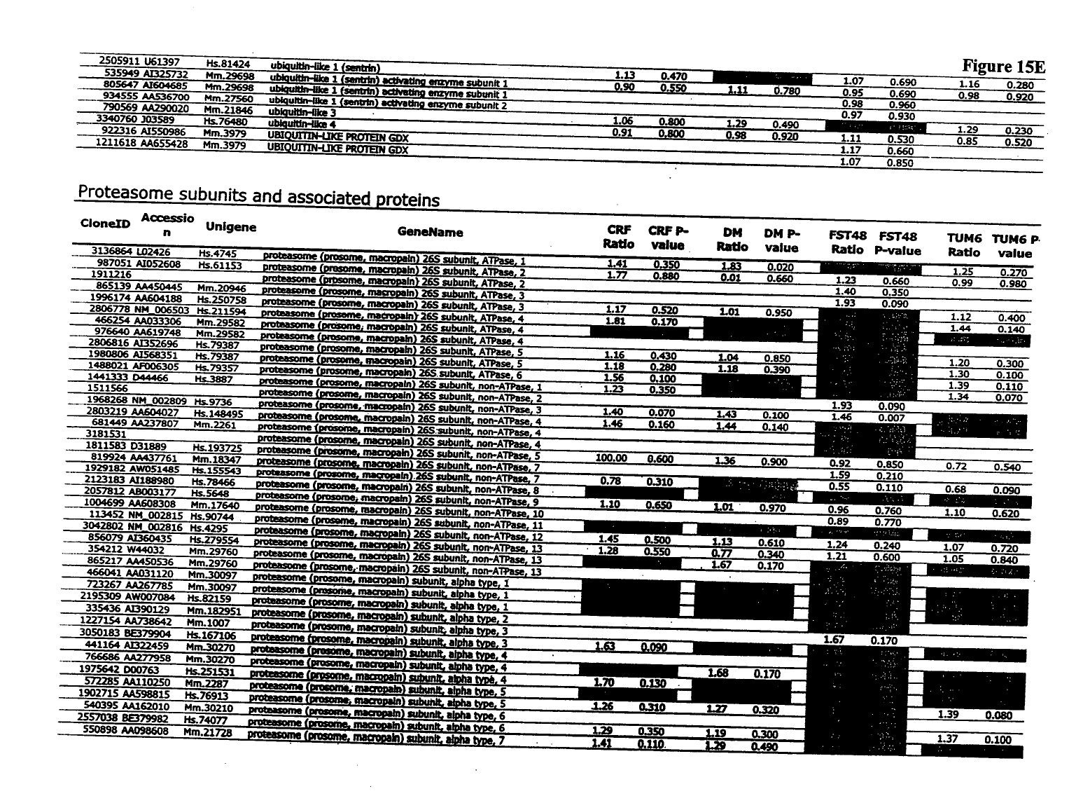

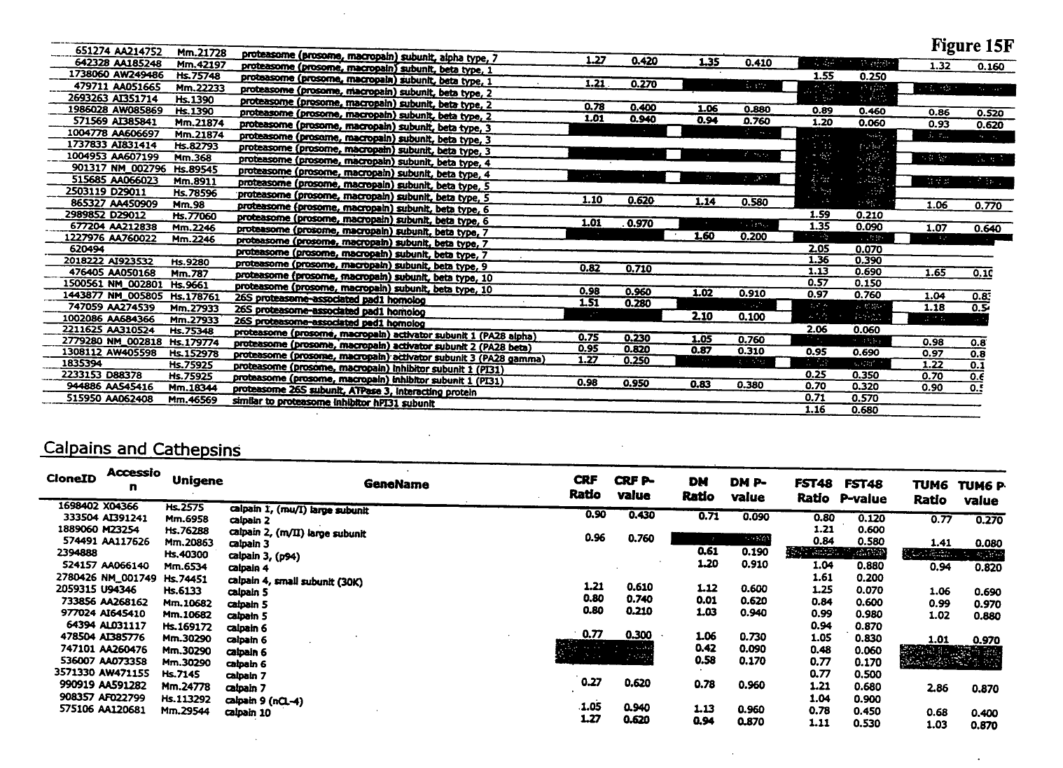

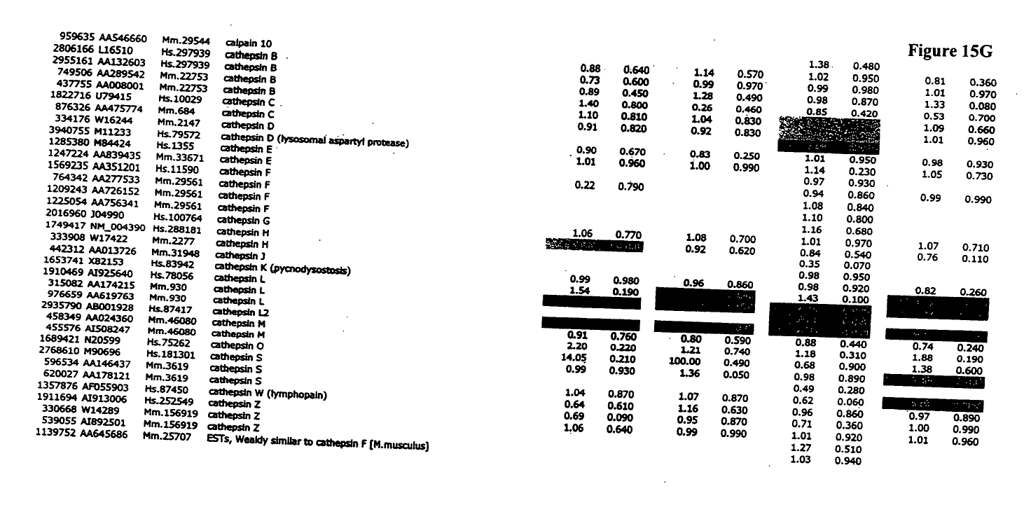

Transcriptional control of atrogins. To test for coordinate regulation of the group of atrogins defined above, we examined˜3 kb of upstream sequences from 31 of the most up-regulated and 17 of the most down-regulated atrogins for common transcription factor binding motifs using the TRANSFAC database (http://transfac.gbf.de/TRANSFAC/). For each of the 124 motifs examined in that database, the frequency of occurrence was calculated and compared between the up- and down-regulated atrogin groups. These results were compared with a group of 16 randomly selected genes that were not differentially expressed in any of the four atrophy states. No motifs were found solely in either up- or down-regulated genes (FIG. 14). The frequency of occurrence of all motifs was similar in genes whose expression was up-regulated, down-regulated or unchanged. Glucocorticoids alone can induce muscle atrophy (69) and appear to be essential for atrophy during fasting, renal failure, and diabetes. We therefore recorded the frequency of glucocorticoid response elements and the frequency of binding sites for other transcription factors, such as Sp1 (70) and C/EBP (71), which are regulated by glucocorticoids and have been proposed to regulate proteasome and Ub expression in atrophying muscle. In the upstream regions of the atrogin genes, the frequency of occurrence of GREs, Sp1 and C/EBP binding sites did not differ among the up-regulated, down-regulated and control groups. Finally, when atrogins were grouped by function (e.g. degradation-related), no difference in frequency of appearance of transcription factor binding motifs was apparent when compared with other atrogins or genes that were not differentially expressed. It remains likely there are as yet undefined transcriptional modulators that activate the changes in gene expression in atrophying muscle.

Discussion

This study is the first to define the pattern of transcriptional changes in muscle in several well-characterized pathological or physiological states that cause muscle wasting. These transcriptional profiles define a set of 120 genes termed atrogins that are consistently up- or down-regulated in catabolic states; together, these adaptations represent a program of changes in mRNA content associated with development of atrophy. Most of these alterations in mRNA content are likely to reflect transcriptional changes, though differences in mRNA degradation rates or mRNA stability may also be contributing to the changes described here. Perhaps the strongest confirmation that this analysis provides valid information about the atrophy process is that many of the genes identified here as atrogins are known to have important functions in muscle wasting, and the ones induced most—atrogin-1/MAFbx and MuRF-1—clearly are essential in this process (19). A number of new and unexpected features of the atrophy process and its regulation are suggested by these atrogins.

Many adaptations enhance capacity for protein degradation. The present findings provide further evidence that the accelerated proteolysis underlying muscle atrophy is due largely to activation of the Ub-proteasome pathway. In fact, increases in mRNAs for polyUb and several proteasome subunits in muscles upon denervation and fasting (2) provided the first clue that different types of atrophy might involve a common set of transcriptional adaptations. This study confirms that mRNAs for polyUb and multiple 26S proteasome subunits rise in atrophying muscles but also demonstrates that mRNAs encoding two Ub ribosomal protein fusion genes, RPS27A and UBA52, generally increase. These Ub fusion proteins had been thought to function constitutively as a source of Ub monomers during growth (72), but since they are induced coordinately with the polyUb genes UBB and UBC, they appear to serve as an additional source of Ub when overall proteolysis rises. Although transcription of several subunits of the 19S and 20S proteasome increase coordinately, some did not change in any catabolic state. Thus, expression of certain proteasome subunits may be subject to tighter transcriptional control than others and may be rate-limiting in the assembly of the mature complex. These findings suggest that in muscle, in contrast to findings in yeast (73), different transcription factors or co-regulators appear to affect the expression of subgroups of proteasome subunits.

Our findings rule out the simplest model for atrophy in which the general acceleration of proteolysis and Ub conjugation results from increased expression of all or many Ub conjugating enzymes in muscle. In fact, mRNAs for the vast majority of Ub-conjugating enzymes did not change (supplementary data, Table 2) whereas mRNAs for two Ub ligases, atrogin-1 and MuRF-1, were dramatically increased. Among the enzymes that did not rise were E2₁₄ₖ (E2A/B) and E3α (Ubr1), which comprise the N-end rule pathway. mRNAs for these factors had been found to increase up to twofold in muscle from fasted, diabetic and tumor-bearing animals (7, 25, 33); the N-end rule pathway has been found to account for most of the increased Ub conjugation in soluble extracts from septic and tumor-bearing animals (24). However, upon fasting, mice lacking E2₁₄ₖ undergo muscle atrophy like control animals (74). mRNA for other E2s has been reported to increase in various models of atrophy [e.g. UbcH2 after TNFα administration in muscle cultures (75) and E2G after glucocorticoid administration to rats (76)]. Our results suggest that these changes are not general features of atrophying muscles, although it is possible that if a larger number of muscles were analyzed, some of these borderline changes (e.g., for E2G, see Supplementary Table 2) would have reached statistical significance.

Suppression of cell growth and extracellular matrix in atrophying muscle. Stimulation of the Akt pathway in muscle causes hypertrophy (57) and suppression of this pathway may trigger atrophy (26, 77). IGF-1-induced hypertrophy occurs via Akt, in all the atrophying muscles studied, mRNA for IGFBP-5, which enhances the effects of IGF-1 (59), fell dramatically, suggesting that activity of the Akt pathway is reduced in these muscles. It is noteworthy that expression of the forkhead transcription factor, Foxo-1 increased in all four catabolic states this family of transcription factors is sequestered and inactivated in the cytoplasm by Akt phosphorylation (78). Reduced Akt phosphorylation would leave Foxo-1 in its under-phosphorylated, active form, which can induce programmed cell death and insulin resistance in several tissues in type II diabetes (48). Insulin resistance is also a prominent feature of muscles in uremia, cancer cachexia, and fasting and should lead to enhanced proteolysis and reduced translation of new proteins (79). Thus, the up-regulation of Foxo-1 may contribute in multiple ways to the atrophy process.

In addition to Foxo1, another proapoptotic gene, Bnip3, was induced˜threefold in all four catabolic states. Bnip3 interacts with Bcl-2 and can antagonize its prosurvival function (60). In the ischemic heart, Bnip3 is induced by hypoxia and acidosis and triggers myocyte death (61). The finding that two proapoptotic genes are also atrogins suggests that both function in each process as part of a growth suppression program.

The extracellular matrix in muscle is generally assumed to be stable. Nevertheless, a rapid decrease in mRNA occurred for many components of the extracellular matrix in these catabolic states. Previous studies have shown a loss of collagen proteins in disuse atrophy (17), and the marked reduction in mRNAs for several extracellular proteins suggests that reduced synthesis of the extracellular matrix is linked to loss of intracellular protein and presumably contractile load.

Control of transcription and translation during atrophy. In addition to the acceleration of overall proteolysis, these systemic forms of muscle atrophy involve a suppression of overall protein synthesis. However, even when protein synthesis is reduced, the atrophying muscles must maintain or even increase the expression of certain key proteins. Indeed, over half of the atrogins are increased in the atrophying muscles at the mRNA level. By contrast, the mRNA for certain transcriptional regulators suggest both activation and repression of gene transcription. Whereas expression of activators such as Foxo1 and MAX are increased, the expression of other proto-oncogenes (growth promotors) such as JUNB (80) fall. Several up-regulated atrogins encode translation initiation factors that could indicate a reduction in translation in muscle. The strong induction of EIF4EBP1, an inhibitor of translation of capped mRNA should reduce overall rates of translation. Simultaneously, mRNA levels for EIF4A2 and EIF4G3 increase, which suggests a mechanism for enhancing translation of the subgroup of mRNAs with internal ribosome entry sites, which tend to be important in stressed cells (53). Together these transcriptional changes indicate ways by which the levels of key proteins may be maintained when overall transcription and translation decrease.

An important regulator of gene expression in muscle that may account for many of the adaptations shown here are glucocorticoids. Excessive secretion of glucocorticoids as occurs in Cushing's syndrome or administering pharmacological doses can cause muscle wasting (81). Adrenal steroid production is required for muscle atrophy in fasting (3), diabetes (27), acidosis (82), and sepsis (83). Among those atrogins induced most dramatically were glutamine synthase and metallothionein, which contain glucocorticoid response elements in their promoter regions (54, 84). However, no GREs were found in the promoters for the majority of up-regulated atrogins including some (e.g. atrogin-1) that are inducible by this hormone. Presumably glucocorticoids act indirectly, perhaps by inducing the expression of a small number of key proteins (e.g. C/EBP), which in turn activate genes induced during atrophy (71).

A reduced circulating level of insulin is a characteristic feature of fasting and type I diabetes, and this lack of insulin can accelerate muscle proteolysis (27). As discussed above, insulin resistance is an important feature of the systemic diseases studied here and probably contributes to muscle wasting. Insulin resistance can be induced by glucocorticoids as well as TNFα, whose production rises in many types of cancer cachexia and sepsis. Induction of the transcription factor Foxo1 may contribute to the insulin resistance characteristic of these atrophying muscles (48).

The present findings raise the possibility that reactive oxygen species play an important general role in atrophy, presumably in initiation or regulation of this response. mRNAs for a thioredoxin-like protein, as well as ATF4 and Nfe2l2, transcription factors that promote expression of oxygen-stress response genes (49, 50), were increased in all these atrophying muscles. Markedly increased were transcripts encoding metallothionein-1 which can play a role in combatting oxidative stress (55). The production of reactive oxygen species has been proposed as a mechanism by which TNFα might damage muscle and regulate gene expression via activation of redox-sensitive transcription factors such as NF-κB (85). Reactive oxygen species produced after burn injury have been proposed to contribute to the loss of muscle at distant sites (86). Clearly, the role of oxygen radicals in atrophy merits in-depth study.

We have studied atrophying muscles in pathophysiological states where systemic muscle wasting is triggered by circulating factors. It remains to be seen whether the group of atrogins identified here also change in denervation and disuse atrophy, where contractile activity is reduced in specific muscles, and where slow-twitch fibers show greatest weight loss. In the systemic diseases studied here, fast-twitch fibers are preferentially lost. While mRNAs for polyub, some proteasome subunits, MuRF-1, and atrogin-1 increase upon denervation/disuse, certain mRNAs found to decrease (IGFBP-5 and parvalbumin) increase upon denervation or unloading (unpublished results refs. 87, 88). It is likely that further important differences will emerge between the wasting induced by systemic diseases and decreased contractile activity.

The demonstration of a transcriptional program in muscle common to a variety of wasting diseases has already suggested novel therapeutic targets to combat muscle wasting (e.g. atrogin-1 and MuRF-1), but has uncovered several unexpected adaptations. A major surprise was that muscle atrophy was not associated with marked reduction in expression of the contractile apparatus. The loss of these components must be due largely to accelerated degradation or reduced translation. A full understanding of the atrophy process will require in-depth analysis of the physiological importance of these responses, especially the decreased expression of glycolytic enzymes or of extracellular matrix, and the induction of transcription factors and proteins related to oxidative stress and NH₃ metabolism. Of obvious importance will be identification of the structure and function of the many other ORFs differentially expressed in these muscles.

### REFERENCES

1. Lecker, S. H., Solomon, V., Mitch, W. E., and Goldberg, A. L. (1999) Muscle protein breakdown and the critical role of the ubiquitin-proteasome pathway in normal and disease states. J. Nutr. 129, 227S-237S
2. Medina, R., Wing, S. S., and Goldberg, A. L. (1995) Increase in levels of polyubiquitin and proteasome mRNA in skeletal muscle during starvation and denervation atrophy. Biochem J. 307, 631-637.
3. Wing, S. S., and Goldberg, A. L. (1993) Glucocorticoids activate the ATP-ubiquitin-dependent proteolytic system in skeletal muscle during fasting. Am. J. Physiol. 264, E668-676
4. Price, S. R., Bailey, J. L., Wang, X., Jurkovitz, C., England, B. K., Ding, X., Phillips, L. S., and Mitch, W. E. (1996) Muscle wasting in insulinopenic rats results from activation of the ATP-dependent, ubiquitin-proteasome proteolytic pathway by a mechanism including gene transcription. J. Clin. Invest. 98, 1703-1708
5. Baracos, V. E., DeVivo, C., Hoyle, D. H. R., and Goldberg, A. L. (1995) Activation of the ATP-ubiquitin-proteasome pathway in skeletal muscle of cachectic rats bearing a hepatoma. Am. J. Physiol. 268, E996-E1006
6. Llovera, M., Garcia-Martinez, C., Agell, N., Marzabal, M., Lopez-Soriano, F. J., and Argiles, J. M. (1994) Ubiquitin gene expression is increased in skeletal muscle of tumour-bearing rats. FEBS Lett. 338, 311-318
7. Temparis, S., Asensi, M., Taillandier, D., Aurousseau, E., Larbaud, D., Obled, A., Bechet, D., Ferrara, M., Estrela, J. M., and Attaix, D. (1994) Increased ATP-ubiquitin-dependent proteolysis in skeletal muscles of tumor-bearing rats. Cancer Res. 54, 5568-5573
8. Mitch, W. E., Medina, R., Grieber, S., May, R. C., England, B. K., Price, S. R., Bailey, J. L., and Goldberg, A. L. (1994) Metabolic acidosis stimulates muscle protein degradation by activating the adenosine triphosphate-dependent pathway involving ubiquitin and proteasomes. J. Clin. Invest. 93, 2127-2133
9. Voisin, L., Breuille, D., Combaret, L., Pouyet, C., Taillandier, D., Aurousseau, E., Obled, C., and Attaix, D. (1996) Muscle wasting in a rat model of long-lasting sepsis results from the activation of lysosomal, Ca²⁺-activated, and ubiquitin-proteasome proteolytic pathways. J. Clin. Invest. 97, 1610-1617.
10. Taillandier, D., Aurousseau, E., Meynial-Denis, D., Bechet, D., Ferrara, M., Cottin, P., Ducastaing, A., Bigard, X., Guezennec, C. Y., Schmid, H. P., and et al. (1996) Coordinate activation of lysosomal, Ca²+-activated and ATP-ubiquitin-dependent proteinases in the unweighted rat soleus muscle. Biochem. J. 316, 65-72.
11. Auclair, D., Garrel, D. R., Chaouki Zerouala, A., and Ferland, L. H. (1997) Activation of the ubiquitin pathway in rat skeletal muscle by catabolic doses of glucocorticoids. Am. J. Physiol. 272, C1007-1016
12. Jagoe, R. T., and Goldberg, A. L. (2001) What do we really know about the ubiquitin-proteasome pathway in muscle atrophy? Curr. Op. Clin. Nutr. Metab. Care 4, 183-190
13. Tiao, G., Hobler, S., Wang, J. J., Meyer, T. A., Luchette, F. A., Fischer, J. E., and Hasselgren, P. O. (1997) Sepsis is associated with increased mRNAs of the ubiquitin-proteasome proteolytic pathway in human skeletal muscle. J. Clin. Invest. 99, 163-168
14. Mansoor, O., Beaufrere, B., Boirie, Y., Ralliere, C., Taillandier, D., Aurousseau, E., Schoeffler, P., Amal, M., and Attaix, D. (1996) Increased mRNA levels for components of the lysosomal, Ca²⁺-activated, and ATP-ubiquitin-dependent proteolytic pathways in skeletal muscle from head trauma patients. Proc. Natl. Acad. Sci. USA 93, 2714-2718
15. Mitch, W. E., and Goldberg, A. L. (1996) Mechanisms of muscle wasting: the role of the ubiquitin-proteasome pathway. N. Engl. J. Med. 335, 1897-1905
16. Gomes, M. D., Lecker, S. H., Jagoe, R. T., Navon, A., and Goldberg, A. L. (2001) Atrogin-1, a muscle-specific F-box protein highly expressed during muscle atrophy. Proc. Natl. Acad. Sci. USA 98, 14440-14445.
17. Cros, N., Tkatchenko, A. V., Pisani, D. F., Leclerc, L., Leger, J. J., Marini, J. F., and Dechesne, C. A. (2001) Analysis of altered gene expression in rat soleus muscle atrophied by disuse. J. Cell. Biochem. 83, 508-519
18. Deval, C., Mordier, S., Obled, C., Bechet, D., Combaret, L., Attaix, D., and Ferrara, M. (2001) Identification of cathepsin L as a differentially expressed message associated with skeletal muscle wasting. Biochem. J. 360, 143-150.
19. Bodine, S. C., Latres, E., Baumhueter, S., Lai, V. K., Nunez, L., Clarke, B. A., Poueymirou, W. T., Panaro, F. J., Na, E., Dharmarajan, K., Pan, Z. Q., Valenzuela, D. M., DeChiara, T. M., Stitt, T. N., Yancopoulos, G. D., and Glass, D. J. (2001) Identification of Ubiquitin Ligases Required for Skeletal Muscle Atrophy. Science 294, 1704-1708
20. Jagoe, R. T., Lecker, S. H., Gomes, M., and Goldberg, A. L. (2002) Patterns of gene expression in atrophying skeletal muscles: the response to food deprivation. FASEB Journal 16, 1697-1712
21. Yechoor, V. K., Patti, M. E., Saccone, R., and Kahn, C. R. (2002) Coordinated patterns of gene expression for substrate and energy metabolism in skeletal muscle of diabetic mice. Proc. Natl. Acad. Sci. USA 99, 10587-10592
22. Tkatchenko, A. V., Pietu, G., Cros, N., Gannoun-Zaki, L., Auffray, C., Leger, J. J., and Dechesne, C. A. (2001) Identification of altered gene expression in skeletal muscles from Duchenne muscular dystrophy patients. Neuromuscul. Disord. 11, 269-277.
23. Bailey, J. L., Wang, X., England, B. K., Price, S. R., Ding, X., and Mitch, W. E. (1996) The acidosis of chronic renal failure activates muscle proteolysis in rats by augmenting transcription of genes encoding proteins of the ATP-dependent ubiquitin-proteasome pathway. J. Clin. Invest. 97, 1447-1453
24. Solomon, V., Baracos, V., Sarraf, P., and Goldberg, A. L. (1998) Rates of ubiquitin conjugation increase when muscles atrophy, largely through activation of the N-end rule pathway. Proc. Natl. Acad. Sci. USA 95, 12602-12607
25. Lecker, S. H., Solomon, V., Price, S. R., Kwon, Y. T., Mitch, W. E., and Goldberg, A. L. (1999) Ubiquitin conjugation by the N-end rule pathway and mRNAs for its components increase in muscles of diabetic rats. J. Clin. Invest. 104, 1411-1420
26. Glass, D. J. (2003) Signalling pathways that mediate skeletal muscle hypertrophy and atrophy. Nat. Cell. Biol. 5, 87-90
27. Mitch, W. E., Bailey, J. L., Wang, X., Jurkovitz, C., Newby, D., and Price, S. R. (1999) Evaluation of signals activating ubiquitin-proteasome proteolysis in a model of muscle wasting. Am. J. Physiol. 276, C1132-1138.
28. Hughes, T. R., Marton, M. J., Jones, A. R., Roberts, C. J., Stoughton, R., Armour, C. D., Bennett, H. A., Coffey, E., Dai, H., He, Y. D., Kidd, M. J., King, A. M., Meyer, M. R., Slade, D., Lum, P. Y., Stepaniants, S. B., Shoemaker, D. D., Gachotte, D., Chakraburtty, K., Simon, J., Bard, M., and Friend, S. H. (2000) Functional discovery via a compendium of expression profiles. Cell 102, 109-126
29. Attaix, D., Taillandier, D., Combaret, L., Ralliere, C., Larbaud, D., Aurousseau, E., and Tanaka, K. (1997) Expression of subunits of the 19S complex and of the PA28 activator in rat skeletal muscle. Mol. Biol. Rep. 24, 95-98.
30. Ustrell, V., Hoffman, L., Pratt, G., and Rechsteiner, M. (2002) PA200, a nuclear proteasome activator involved in DNA repair. EMBO J. 21, 3516-3525
31. Borodovsky, A., Kessler, B. M., Casagrande, R., Overkleeft, H. S., Wilkinson, K. D., and Ploegh, H. L. (2001) A novel active site-directed probe specific for deubiquitylating enzymes reveals proteasome association of USP14. EMBO J. 20, 5187-5196
32. Wray, C. J., Mammen, J. M., Hershko, D. D., and Hasselgren, P. O. (2003) Sepsis upregulates the gene expression of multiple ubiquitin ligases in skeletal muscle. Int. J. Biochem. Cell Biol. 35, 698-705
33. Wing, S. S., and Banville, D. (1994) 14-kDa ubiquitin-conjugating enzyme: structure of the rat gene and regulation upon fasting and by insulin. Am. J. Physiol. 267, E39-48
34. Kwon, Y. T., Xia, Z., Davydov, I. V., Lecker, S. H., and Varshavsky, A. (2001) Construction and Analysis of Mouse Strains Lacking the Ubiquitin Ligase UBR1 (E3alpha) of the N-End Rule Pathway. Mol. Cell Biol. 21, 8007-8021.
35. Fischer, D., Sun, X., Gang, G., Pritts, T., and Hasselgren, P. O. (2000) The gene expression of ubiquitin ligase E3alpha is upregulated in skeletal muscle during sepsis in rats-potential role of glucocorticoids. Biochem. Biophys. Res. Commun. 267, 504-508
36. Hatakeyama, S., Yada, M., Matsumoto, M., Ishida, N., and Nakayama, K. I. (2001) U box proteins as a new family of ubiquitin-protein ligases. J. Biol. Chem. 276, 33111-33120
37. Lester, D., Farquharson, C., Russell, G., and Houston, B. (2000) Identification of a family of noncanonical ubiquitin-conjugating enzymes structurally related to yeast UBC6. Biochem. Biophys. Res. Commun. 269, 474-480
38. Takahashi, H., Ishidoh, K., Muno, D., Ohwada, A., Nukiwa, T., Kominami, E., and Kira, S. (1993) Cathepsin L activity is increased in alveolar macrophages and bronchoalveolar lavage fluid of smokers. Am. Rev. Repir. Dis. 147, 1562-1568
39. Balcerzak, D., Querengesser, L., Dixon, W. T., and Baracos, V. E. (2001) Coordinate expression of matrix-degrading proteinases and their activators and inhibitors in bovine skeletal muscle. J. Anim. Sci. 79, 94-107
40. Reznick, A. Z., Menashe, O., Bar-Shai, M., Coleman, R., and Carmeli, E. (2003) Expression of matrix metalloproteinases, inhibitor, and acid phosphatase in muscles of immobilized hindlimbs of rats. Muscle Nerve 27, 51-59
41. Schoser, B. G., and Blottner, D. (1999) Matrix metalloproteinases MMP-2, MMP-7 and MMP-9 in denervated human muscle. Neuroreport 10, 2795-2797
42. Baracos, V. E. (2002) Hypercatabolism and hypermetabolism in wasting states. Curr. Opin. Clin. Nutr. Metab. Care 5, 237-239
43. Chesney, J., Mitchell, R., Benigni, F., Bacher, M., Spiegel, L., Al-Abed, Y., Han, J. H., Metz, C., and Bucala, R. (1999) An inducible gene product for 6-phosphofructo-2-kinase with an AU-rich instability element: role in tumor cell glycolysis and the Warburg effect. Proc. Natl. Acad. Sci. USA 96, 3047-3052
44. Lowenstein, J. M., and Goodman, M. N. (1978) The purine nucleotide cycle in skeletal muscle. Fed Proc 37, 2308-2312
45. Karinch, A. M., Pan, M., Lin, C. M., Strange, R., and Souba, W. W. (2001) Glutamine metabolism in sepsis and infection. J. Nutr. 131, 2535S-2538S; discussion 2550S-2531S
46. Pineda, M., Fernandez, E., Torrents, D., Estevez, R., Lopez, C., Camps, M., Lloberas, J., Zorzano, A., and Palacin, M. (1999) Identification of a membrane protein, LAT-2, that Co-expresses with 4F2 heavy chain, an L-type amino acid transport activity with broad specificity for small and large zwitterionic amino acids. J. Biol. Chem. 274, 19738-19744
47. Bode, B. P. (2001) Recent molecular advances in mammalian glutamine transport. J. Nutr. 131, 2475S-2485S; discussion 2486S-2477S
48. Nakae, J., Biggs, W. H., Kitamura, T., Cavenee, W. K., Wright, C. V., Arden, K. C., and Accili, D. (2002) Regulation of insulin action and pancreatic beta-cell function by mutated alleles of the gene encoding forkhead transcription factor Foxo1. Nat. Genet. 32, 245-253
49. Chan, K., Han, X. D., and Kan, Y. W. (2001) An important function of Nrf2 in combating oxidative stress: detoxification of acetaminophen. Proc. Natl. Acad. Sci. USA 98, 4611-4616
50. Harding, H. P., Zhang, Y., Zeng, H., Novoa, I., Lu, P. D., Calfon, M., Sadri, N., Yun, C., Popko, B., Paules, R., Stojdl, D. F., Bell, J. C., Hettmann, T., Leiden, J. M., and Ron, D. (2003) An integrated stress response regulates amino Acid metabolism and resistance to oxidative stress. Mol. Cell 11, 619-633
51. Bertolino, E., Reimund, B., Wildt-Perinic, D., and Clerc, R. G. (1995) A novel homeobox protein which recognizes a TGT core and functionally interferes with a retinoid-responsive motif. J. Biol. Chem. 270, 31178-31188
52. Goldring, K., Jones, G. E., Thiagarajah, R., and Watt, D. J. (2002) The effect of galectin-1 on the differentiation of fibroblasts and myoblasts in vitro. J. Cell. Sci. 115, 355-366
53. Vagner, S., Galy, B., and Pyronnet, S. (2001) Irresistible IRES: Attracting the translation machinery to internal ribosome entry sites. EMBO Rep. 2, 893-898.
54. Kelly, E. J., Sandgren, E. P., Brinster, R. L., and Palmiter, R. D. (1997) A pair of adjacent glucocorticoid response elements regulate expression of two mouse metallothionein genes. Proc. Natl. Acad. Sci. USA 94, 10045-10050.
55. Nath, R., Kumar, D., Li, T., and Singal, P. K. (2000) Metallothioneins, oxidative stress and the cardiovascular system. Toxicology 155, 17-26.
56. Lee, K. K., Murakawa, M., Takahashi, S., Tsubuki, S., Kawashima, S., Sakamaki, K., and Yonehara, S. (1998) Purification, molecular cloning, and characterization of TRP32, a novel thioredoxin-related mammalian protein of 32 kDa. J. Biol. Chem. 273, 19160-19166
57. Musaro, A., McCullagh, K. J., Naya, F. J., Olson, E. N., and Rosenthal, N. (1999) IGF-1 induces skeletal myocyte hypertrophy through calcineurin in association with GATA-2 and NF-ATc1. Nature 400, 581-585
58. Bodine, S. C., Stitt, T. N., Gonzalez, M., Kline, W. O., Stover, G. L., Bauerlein, R., Zlotchenko, E., Scrimgeour, A., Lawrence, J. C., Glass, D. J., and Yancopoulos, G. D. (2001) Akt/mTOR pathway is a crucial regulator of skeletal muscle hypertrophy and can prevent muscle atrophy in vivo. Nat. Cell Biol. 3, 1014-1019.
59. Schneider, M. R., Wolf, E., Hoeflich, A., and Lahm, H. (2002) IGF-binding protein-5: flexible player in the IGF system and effector on its own. J. Endocrinol. 172, 423-440
60. Boyd (1994) Adenovirus EIB 19 kDa and Bcl-2 proteins interact with a common set of cellular proteins. Cell 79, 1121
61. Kubasiak, L. A., Hernandez, O. M., Bishopric, N. H., and Webster, K. A. (2002) Hypoxia and acidosis activate cardiac myocyte death through the Bcl-2 family protein BNIP3. Proc. Natl. Acad. Sci. USA 99, 12825-12830
62. Nishimoto, S., Hamajima, Y., Toda, Y., Toyoda, H., Kitamura, K., and Komurasaki, T. (2002) Identification of a novel smooth muscle associated protein, smap2, upregulated during neointima formation in a rat carotid endarterectomy model. Biochim. Biophys. Acta 1576, 225-230
63. Zhou, Q., Ruiz-Lozano, P., Martone, M. E., and Chen, J. (1999) Cypher, a striated muscle-restricted PDZ and LIM domain-containing protein, binds to alpha-actinin-2 and protein kinase C. J. Biol. Chem. 274, 19807-19813
64. Peterfy, M., Phan, J., Xu, P., and Reue, K. (2001) Lipodystrophy in the fld mouse results from mutation of a new gene encoding a nuclear protein, lipin. Nat. Genet. 27, 121-124
65. Huffman, T. A., Mothe-Satney, I., and Lawrence, J. C., Jr. (2002) Insulin-stimulated phosphorylation of lipin mediated by the mammalian target of rapamycin. Proc. Natl. Acad. Sci. USA 99, 1047-1052
66. Guardavaccaro, D., Ciotti, M. T., Schafer, B. W., Montagnoli, A., and Tirone, F. (1995) Inhibition of differentiation in myoblasts deprived of the interferon-related protein PC4. Cell Growth Differ. 6, 159-169
67. Mariani, L., McDonough, W. S., Hoelzinger, D. B., Beaudry, C., Kaczmarek, E., Coons, S. W., Giese, A., Moghaddam, M., Seiler, R. W., and Berens, M. E. (2001) Identification and validation of P311 as a glioblastoma invasion gene using laser capture microdissection. Cancer Res. 61, 4190-4196
68. Pan, D., Zhe, X., Jakkaraju, S., Taylor, G. A., and Schuger, L. (2002) P311 induces a TGF-betal-independent, nonfibrogenic myofibroblast phenotype. J. Clin. Invest. 110, 1349-1358
69. Hasselgren, P. O. (1999) Glucocorticoids and muscle catabolism. Curr. Opin. Clin. Nutr. Metab. Care 2, 201-205
70. Marinovic, A. C., Zheng, B., Mitch, W. E., and Price, S. R. (2002) Ubiquitin (UbC) expression in muscle cells is increased by glucocorticoids through a mechanism involving Sp1 and MEK1. J. Biol. Chem. 277, 16673-16681
71. Penner, G., Gang, G., Sun, X., Wray, C., and Hasselgren, P. O. (2002) C/EBP DNA-binding activity is upregulated by a glucocorticoid-dependent mechanism in septic muscle. Am. J. Physiol. Regul. Integr. Comp. Physiol. 282, R439-444
72. Finley, D., Bartel, B., and Varshavsky, A. (1989) The tails of ubiquitin precursors are ribosomal proteins whose fusion to ubiquitin facilitates ribosome biogenesis. Nature 338, 394-401
73. Mannhaupt, G., Schnall, R., Karpov, V., Vetter, I., and Feldmann, H. (1999) Rpn4p acts as a transcription factor by binding to PACE, a nonamer box found upstream of 26S proteasomal and other genes in yeast. FEBS Lett. 450, 27-34
74. Adegoke, O. A., Bedard, N., Roest, H. P., and Wing, S. S. (2002) Ubiquitin-conjugating enzyme E214k/HR6B is dispensable for increased protein catabolism in muscle of fasted mice. Am. J. Physiol. Endocrinol. Metab. 283, E482-489
75. Li, Y. P., Lecker, S. H., Chen, Y., Waddell, I. D., Goldberg, A. L., and Reid, M. B. (2003) TNF-alpha increases ubiquitin-conjugating activity in skeletal muscle by up-regulating UbcH2/E220k. FASEB J. 17, 1048-1057
76. Chrysis, D., and Underwood, L. E. (1999) Regulation of components of the ubiquitin system by insulin-like growth factor I and growth hormone in skeletal muscle of rats made catabolic with dexamethasone. Endocrinology 140, 5635-5641
77. Rommel, C., Bodine, S. C., Clarke, B. A., Rossman, R., Nunez, L., Stitt, T. N., Yancopoulos, G. D., and Glass, D. J. (2001) Mediation of IGF-1-induced skeletal myotube hypertrophy by PI (3)K/Akt/mTOR and PI (3)K/Akt/GSK3 pathways. Nat. Cell Biol. 3, 1009-1013.
78. Datta, S. R., Brunet, A., and Greenberg, M. E. (1999) Cellular survival: a play in three Akts. Genes Dev. 13, 2905-2927
79. Zierath, J. R., Krook, A., and Wallberg-Henriksson, H. (2000) Insulin action and insulin resistance in human skeletal muscle. Diabetologia 43, 821-835
80. Mathas, S., Hinz, M., Anagnostopoulos, I., Krappmann, D., Lietz, A., Jundt, F., Bommert, K., Mechta-Grigoriou, F., Stein, H., Dorken, B., and Scheidereit, C. (2002) Aberrantly expressed c-Jun and JunB are a hallmark of Hodgkin lymphoma cells, stimulate proliferation and synergize with NF-kappa B. EMBO J. 21, 4104-4113
81. Kayali, A. G., Young, V. R., and Goodman, M. N. (1987) Sensitivity of myofibrillar proteins to glucocorticoid-induced muscle proteolysis. Am. J. Physiol. 252, E621-626
82. Price, S. R., England, B. K., Bailey, J. L., Van Vreede, K., and Mitch, W. E. (1994) Acidosis and glucocorticoids concomitantly increase ubiquitin and proteasome subunit mRNAs in rat muscle. Am. J. Physiol. 267, C955-960
83. Tiao, G., Fagan, J., Roegner, V., Lieberman, M., Wang, J. J., Fischer, J. E., and Hasselgren, P. O. (1996) Energy-ubiquitin-dependent muscle proteolysis during sepsis in rats is regulated by glucocorticoids. J. Clin. Invest. 97, 339-348
84. Chandrasekhar, S., Souba, W. W., and Abcouwer, S. F. (1999) Identification of glucocorticoid-responsive elements that control transcription of rat glutamine synthetase. Am. J. Physiol. 276, L319-331.
85. Reid, M. B., and Li, Y. P. (2001) Cytokines and oxidative signalling in skeletal muscle. Acta Physiol. Scand. 171, 225-232
86. Fagan, J. M., Ganguly, M., Stockman, H., Ferland, L. H., and Toner, M. (1999) Posttranslational modifications of cardiac and skeletal muscle proteins by reactive oxygen species after burn injury in the rat. Ann Surg. 229, 106-114
87. Olive, M., and Ferrer, I. (1994) Parvalbumin immunohistochemistry in denervated skeletal muscle. Neuropathol. Appl. Neurobiol. 20, 495-500

88. Bayol, S., Loughna, P. T., and Brownson, C. (2000) Phenotypic expression of IGF binding protein transcripts in muscle, in vitro and in vivo. Biochem. Biophys. Res. Commun. 273, 282-286

TABLE 1
Loss of muscle weight and increases in muscle protein degradation in the catabolic
states studied

| | | Muscle weight | | Protein degradation | |
|---|---|---|---|---|---|
| Condition | Sample | (mg) | % loss | (pmol Tyr mg⁻¹ 2 h⁻¹) | % increase |
| Fasting^{a} | Control | 137.3 ± 1.8 | | 230 ± 11 | |
| | Fasting | 118.4 ± 1.7 | 14 | 342 ± 13 | 49 |
| Tumor implantation^{b} | Control | 45.0 ± 0.6 | | 251 ± 13 | |
| (Yoshida hepatoma) | Tumor | 38.6 ± 0.7 | 14 | 410 ± 17 | 63 |
| Chronic renal failure^{c} | Control | 28.6 ± 1.4 | | 150 ± 13 | |
| (7/8 nephrectomy) | CRF | 20.3 ± 1.5 | 29 | 236 ± 32 | 57 |
| Diabetes^{c} | Control | 39.4 ± 1.0 | | 149 ± 5 | |
| (streptozotocin) | Diabetes | 31.6 ± 1.1 | 20 | 208 ± 8 | 40 |

^{a}Differences in muscle weights refer to changes in mouse gastrocnemius after 48 hours fast, as reported previously (20). Protein degradation rates taken from (3) refer to EDL muscle in rat after 24 h fasting.
^{b}Muscle weights and degradation rates measured in epitrochlearis muscle in rats 5 days after implantation of Yoshida ascites hepatoma and taken from ref. (5). Similar weight changes were described for gastrocnemius (5). Even greater weight loss (22%) occurred in the lateral gastrocnemius muscles used in the present study.
^{c}Representative values for muscle weights and protein degradation rates measured in rat epitrochlearis muscle used in the present study and similar to published results for chronic renal failure (23) and diabetes (4).

TABLE 2
Number of coordinately regulated genes in atrophying muscles^{a}

| | Atrogins^{b} | | Shared^{c} | | | | | |
|---|---|---|---|---|---|---|---|---|
| | (common to all | | (2 or 3 | | Disease-specific^{d} | | | |
| | states) | | states) | | (single state) | | Unchanged^{e} | |
| | N | (%) | N | (%) | N | (%) | N | (%) |
| Fasting | 133 | (0.8) | 631 | (3.8) | 164 | (1.0) | 14635 | (89.3) |
| Diabetes | | | 223 | (1.4) | 7 | (0.04) | 15859 | (96.7) |
| Uremia | | | 635 | (3.9) | 31 | (0.2) | 15269 | (93.1) |
| Tumor | | | 719 | (4.4) | 40 | (0.2) | 15069 | (91.9) |

^{a}Numbers of genes grouped by differential expression pattern for all 16, 392 sequences giving analysable results in all four states in both human and mouse cDNA microarrays. Differential expression was based on P value from the Rosetta error model for Incyte microarrays:
^{b}P < 0.05 in all states;
^{c}P < 0.05 in that state and 1 or 2 others;
^{d}P < 0.005 in that state and >0.02 in all other states;
^{e}P >= 0.05

| FIG. | CloneID | Accession Number | Unigene | Cluster ID |
|---|---|---|---|---|
| 3 | 1685342 | U62961 | Hs.177584 | OXCT |
| 3 | 640734 | AA203878 | Mm.19669 | Pfkfb3 |
| 4 | 2916261 | NM_001675 | Hs.181243 | ATF4 |
| 3 | 3034694 | J03592 | Hs.164280 | SLC25A6 |
| 3 | 750480 | AA389897 | Mm.3238 | Ampd3 |
| 3 | 3206210 | AW161540 | Hs.155101 | ATP5A1 |
| 1 | 1723142 | AW051824 | Hs.61661 | FBXO32 |
| 6 | 463388 | AA030640 | Mm.2159 | Bnip3 |
| 6 | 571367 | AA105295 | Mm.2159 | Bnip3 |
| 6 | 2153373 | X51405 | Hs.75360 | CPE |
| 6 | 1858644 | AI936438 | Hs.82201 | CSNK2A2 |
| 1 | 315082 | AA174215 | Mm.930 | Ctsl |
| 1 | 2935790 | AB001928 | Hs.87417 | CTSL2 |
| 3 | 903905 | AA521755 | Mm.27830 | Slc7a8 |
| 5 | 782235 | AW577407 | Hs.172928 | COL1A1 |
| 5 | 420322 | W89883 | Mm.147387 | Col3a1 |
| 5 | 1887959 | Y14690 | Hs.82985 | COL5A2 |
| 5 | 4287342 | L01697 | Hs.83164 | COL15A1 |
| 3 | 57382 | J05401 | Hs.80691 | CKMT2 |
| 4 | 2964704 | NM_001991 | Hs.194669 | EZH1 |
| 3 | 316967 | W11965 | Mm.29994 | Eno3 |
| n | 472432 | | Mm. | |
| n | 478025 | AA049360 | Mm.35811 | |
| n | 692699 | AA239666 | Mm.41583 | |
| n | 719597 | AA255088 | Mm.21963 | |
| n | 761596 | AA272340 | Mm.38231 | |
| n | 806740 | AA403565 | Mm.34459 | |
| n | 2202945 | AW173127 | Hs.30120 | |
| n | 809255 | AA445144 | Mm.6720 | |
| 5 | 2060081 | AL117412 | Hs.173912 | EIF4A2 |
| 5 | 2504983 | BE257720 | Hs.71819 | EIF4EBP1 |
| 5 | 2266829 | Z34918 | Hs.25732 | EIF4G3 |
| 5 | 1448051 | X63556 | Hs.750 | FBN1 |
| 5 | 3553729 | X02761 | Hs.287820 | FN1 |
| 4 | 334077 | W36356 | Mm.29891 | Foxo1 |
| 6 | 775912 | AA276338 | Mm.14638 | Gabarapl1 |
| 5 | 2495131 | AA035793 | Hs.227751 | LGALS1 |
| 3 | 440344 | AA011759 | Mm.2338 | Glns |
| 3 | 693146 | AI466979 | Mm.2338 | Glns |
| 3 | 570675 | AA108640 | Mm.10669 | Gdc1 |
| n | 62790 | NM_005493 | Hs.306242 | |
| n | 2299686 | AL136632 | | |
| n | 660932 | AA216947 | Mm.205737 | |
| n | 679931 | AA237701 | Mm.22749 | |
| n | 986558 | AI090186 | Hs.17379 | |
| 3 | 2056855 | NM_000884 | Hs.75432 | IMPDH2 |
| 5 | 671661 | AA241784 | Mm.578 | Igfbp5 |
| 6 | 1833174 | AI183499 | Hs.17109 | ITM2A |
| 6 | 1752254 | NM_001550 | Hs.7879 | IFRD1 |
| 4 | 2819825 | U20734 | Hs.198951 | JUNB |
| 1 | 448976 | D38521 | Hs.112396 | PA200 |
| 3 | 1335140 | X02152 | Hs.2795 | LDHA |
| 6 | 336726 | AI390969 | Mm.29733 | Ldb3 |
| 6 | 581906 | AA154452 | Mm.28548 | Lpln1 |
| 3 | 487539 | AA314267 | Hs.75375 | MDH1 |
| 3 | 318346 | W13686 | Mm.3156 | Mor2 |
| 4 | 2579965 | AI693307 | Hs.42712 | MAX |
| 6 | 480068 | AA051654 | | Mt1 |
| 6 | 2048551 | R99207 | Hs.36102 | SMHU1B |
| 6 | 2513883 | F26137 | | MT1L |
| 6 | 889720 | AA498356 | Mm.28357 | Map1lc3 |
| 6 | 475803 | AA050417 | Mm.4048 | Myd116 |
| 6 | 972224 | M21812 | Hs.50889 | HUMMLC2B |
| 6 | 2118695 | NM_002477 | Hs.170482 | MYL5 |
| 3 | 671212 | AA222463 | Mm.28058 | Ndufb5 |
| 3 | 404593 | W83085 | Mm.2060 | |
| 3 | 367925 | W54068 | Mm.1893 | |
| 3 | 119068 | NM_005006 | Hs.8248 | NDUFS1 |
| 3 | 83825 | AI557288 | Hs.51299 | NDUFV2 |
| 4 | 475505 | AA044475 | Mm.1025 | Nfe2l2 |
| 5 | 1930884 | AK000250 | Hs.79110 | NCL |
| 5 | 403071 | W81878 | Mm.10681 | Osf2 |
| 5 | 1994715 | NM_006475 | Hs.136348 | OSF2 |
| 6 | 374970 | W62819 | Mm.4919 | p311 |
| 6 | 425866 | AA000945 | Mm.4919 | P311 |
| 6 | 733420 | AA259807 | Mm.4919 | P311 |
| 6 | 1555545 | NM_004772 | Hs.142827 | P311 |
| 6 | 420049 | W91158 | Mm.29742 | Pa26 |
| 6 | 2289252 | AI022812 | Hs.295449 | PVALB |
| 3 | 3032691 | BE267587 | Hs.181013 | PGAM1 |
| 3 | 3028411 | M55674 | Hs.46039 | PGAM2 |
| 3 | 2952043 | X80590 | Hs.54929 | PHKG1 |
| 5 | 1672920 | AF053356 | Hs.202097 | PCOLCE |
| 1 | 466254 | AA033306 | Mm.29582 | Psmc4 |
| 1 | 2123183 | AI188980 | Hs.78466 | PSMD8 |
| 1 | 113452 | NM_002815 | Hs.90744 | PSMD11 |
| 1 | 833508 | AA465980 | Mm.28571 | Psmd11 |
| 1 | 466041 | AA031120 | Mm.30097 | Psma1 |
| 1 | 723267 | AA267785 | Mm.30097 | Psma1 |
| 1 | 2195309 | AW007084 | Hs.82159 | PSMA1 |
| 1 | 572285 | AA110250 | Mm.2287 | Psma5 |
| 1 | 571569 | AI385841 | Mm.21874 | Psmb3 |
| 1 | 1737833 | AI831414 | Hs.82793 | PSMB3 |
| 1 | 901317 | NM_002796 | Hs.89545 | PSMB4 |
| 3 | 422907 | W97904 | Mm.38786 | PDC-E2 |
| 3 | 2500366 | Y00978 | Hs.115285 | PDC-E2 |
| 5 | 2132508 | NM_000976 | Hs.182979 | RPL12 |
| 1 | 2132619 | AA583480 | Hs.3297 | RPS27A |
| n | 480920 | AI595466 | Mm.41792 | |
| n | 747941 | AA260950 | Mm.30000 | |
| n | 763553 | AA285513 | Mm.28654 | |
| n | 747297 | AA274938 | Mm.17880 | |
| 6 | 315676 | W09957 | Mm.25311 | |
| 6 | 621966 | AA183749 | Mm.25311 | |
| 6 | 651825 | AA212204 | Mm.25311 | |
| n | 1938951 | AI536745 | Hs.82273 | |
| n | 847202 | AA420054 | Mm.24619 | |
| n | 807957 | AA432925 | Mm.29592 | |
| n | 634167 | AA182068 | Mm.29181 | |
| n | 961271 | AA547550 | Mm.180499 | |
| n | 791051 | AA415219 | Mm.25119 | |
| n | 619799 | AA172929 | Mm.24482 | |
| n | 680307 | AI451825 | Mm.40897 | |
| 5 | 2757583 | AI814448 | Hs.8765 | RNAHP |
| 5 | 482198 | AA059909 | Mm.30162 | Smoc2 |
| 5 | 735413 | AA272826 | Mm.30162 | Smoc2 |
| 4 | 642177 | AA212233 | Mm.200168 | Srcap |
| 6 | 574914 | AA120631 | Mm.157511 | Slc20a2 |
| 3 | 63038 | Z14136 | Hs.28491 | SAT |
| 6 | 445988 | AA020051 | Mm.15125 | Sdfr1 |
| 6 | 618630 | AA174980 | Mm.22421 | Tebp |
| 4 | 722623 | AA260654 | Mm.8155 | Tgif |
| 6 | 938004 | AF051896 | Hs.18792 | TXNL |
| 6 | 403681 | W82203 | Mm.28683 | Trf1 |
| 3 | 2832214 | U47924 | Hs.83848 | TPI1 |
| 1 | 4157922 | AF075321 | Hs.5308 | UBA52 |
| 1 | 3137251 | U49869 | Hs.183842 | UBB |
| 1 | 2730250 | AA599258 | Hs.183704 | UBC |
| 1 | 751477 | AA395996 | Mm.32920 | Ncube1 |
| 1 | 1707220 | U30888 | Hs.75981 | USP14 |
| 1 | 747318 | AA274945 | Mm.21634 | Ube4b |
| 4 | 2648611 | AF055376 | Hs.30250 | MAF |

GeneName

3-oxoacid CoA transferase
6-phosphofructo-2-kinase, inducible
activating transcription factor 4 (tax-responsive enhancer element B67)
adenine nucleotide translocator (solute carrier family 25), member 6
AMP deaminase 3
ATP synthase, H+ transporting, mitochondrial F1 complex, alpha subunit,
isoform 1, cardiac muscle
atrogin-1/MAFbx1/Fbxo32
BCL2/adenovirus E1B 19 kDa-interacting protein 1, NIP3
BCL2/adenovirus E1B 19 kDa-interacting protein 1, NIP3
carboxypeptidase E
casein kinase 2, alpha prime polypeptide
cathepsin L
cathepsin L2
cationic amino acid transporter, y+ system
(solute carrier family 8), member 7
collagen, type I, alpha 1
collagen, type III, alpha 1
collagen, type V, alpha 2
collagen, type XV, alpha 1
creatine kinase, mitochondrial 2 (sarcomeric)
enhancer of zeste homolog 1
enolase 3, beta muscle
ESTs
ESTs
ESTs
ESTs
ESTs
ESTs
ESTs
ESTs, highly similar to similar to hypothetical protein FLJ20038
eukaryotic translation initiation factor 4A, isoform 2
eukaryotic translation initiation factor 4E binding protein 1
eukaryotic translation initiation factor 4G, isoform 3
fibrillin 1
fibronectin 1
forkhead box O1 (FKHR1)
GABA(A) receptor-associated protein like 1
galectin 1 (lectin, galactoside-binding, soluble, 1)
glutamine synthetase
glutamine synthetase
glycerol phosphate dehydrogenase 1, cytoplasmic adult
Homo sapiens KB07 protein mRNA, partial cds
hypothetical protein FLJ12619
hypothetical protein MGC12070, C-terminus weakly similar to E3alpha
hypothetical protein MGC7474
hypothetical protein T42683
IMP (inosine monophosphate) dehydrogenase 2
insulin-like growth factor binding protein 5
integral membrane protein 2A
interferon-related developmental regulator 1
jun B proto-oncogene
KIAA0077 protein
lactate dehydrogenase A
LIM-binding domain 3 (Z-band alternatively spliced PDZ-motif protein)
lipin 1
malate dehydrogenase 1, NAD (soluble)
malate dehydrogenase, soluble
MAX protein (myc-associated protein X)
metallothionein 1
metallothionein 1B
metallothionein 1L
microtubule-associated protein 1 light chain 3
myeloid differentiation primary response gene 116
myosin light chain 2
myosin, light polypeptide 5, regulatory
NADH dehydrogenase (ubiquinone) 1 beta subcomplex 5
NADH dehydrogenase (ubiquinone) 1 beta subcomplex, 8 (19 kD)
NADH dehydrogenase (ubiquinone) 1, subcomplex unknown, 2 (14.5 kD)
NADH dehydrogenase (ubiquinone) Fe—S protein 1 (75 kD)
(NADH-coenzyme Q reductase)
NADH dehydrogenase (ubiquinone) flavoprotein 2 (24 kD)
nuclear, factor, erythroid derived 2, like 2
nucleolin
osteoblast specific factor 2 (fasciclin I-like)
osteoblast specific factor 2 (fasciclin I-like)
P311
P311
P311
P311
p53 regulated PA26 nuclear protein
parvalbumin
phosphoglycerate mutase 1 (brain)
phosphoglycerate mutase 2 (muscle)
phosphorylase kinase, gamma 1 (muscle)
procollagen C-endopeptidase enhancer
proteasome (prosome, macropain) 19S subunit, ATPase, 4
proteasome (prosome, macropain) 19S subunit, non-ATPase, 08
proteasome (prosome, macropain) 19S subunit, non-ATPase, 11
proteasome (prosome, macropain) 19S subunit, non-ATPase, 11
proteasome (prosome, macropain) 20S subunit, alpha type, 1
proteasome (prosome, macropain) 20S subunit, alpha type, 1
proteasome (prosome, macropain) 20S subunit, alpha type, 1
proteasome (prosome, macropain) 20S subunit, alpha type, 5
proteasome (prosome, macropain) 20S subunit, beta type, 3
proteasome (prosome, macropain) 20S subunit, beta type, 3
proteasome (prosome, macropain) 20S subunit, beta type, 4
pyruvate dehydrogenase complex, E2 component
pyruvate dehydrogenase complex, E2 component
ribosomal protein L12
ribosomal protein S27a
RIKEN cDNA 0610011B16 gene
RIKEN cDNA 1110029F20 gene
RIKEN cDNA 1300003P13 gene
RIKEN cDNA 1700027M01 gene
RIKEN cDNA 1810015C04 gene
RIKEN cDNA 1810015C04 gene
RIKEN cDNA 1810015C04 gene
RIKEN cDNA 1810015C04 gene (hypothetical protein FLJ20152)
RIKEN cDNA 2310042D19 gene
RIKEN cDNA 2410127L17 gene
RIKEN cDNA 2500002K03 gene
RIKEN cDNA 2610029G23 gene
RIKEN cDNA 3230401D17 gene
RIKEN cDNA 5730460C18 gene
RIKEN cDNA 9130022E05 gene
RNA helicase-related protein
secreted modular calcium-binding protein 2
secreted modular calcium-binding protein 2
Snf2-related CBP activating protein
solute carrier family 20, member 2
spermidine/spermine N1-acetyltransferase
stromal cell derived factor receptor 1
telomerase binding protein, p23 (p23 unactive progesterone
receptor, cochaperone)
TG interacting factor
thioredoxin-like, 32 kD
transferrin receptor
triosephosphate isomerase 1
ubiquitin A-52 residue ribosomal protein fusion product 1
ubiquitin B
ubiquitin C
ubiquitin conjugating enzyme 1, non-canonical
ubiquitin specific protease 14
ubiquitination factor E4B
v-maf musculoaponeurotic fibrosarcoma oncogene homolog

| FST48 | FST48 | TUM6 | TUM6 | CRF | | | |
|---|---|---|---|---|---|---|---|
| Ratio | P-value | Ratio | P-value | Ratio | CRF P-value | DM Ratio | DM P-value |
| 0.560 | 0.00171 | 0.470 | 0.02000 | 0.480 | 0.00070 | 0.480 | 0.01000 |
| 0.670 | 0.00881 | 4.060 | 0.00000 | 3.790 | 0.00008 | 4.460 | 0.00252 |
| 1.990 | 0.00001 | 1.890 | 0.00002 | 2.010 | 0.00001 | 1.900 | 0.00011 |
| 0.660 | 0.00172 | 0.660 | 0.04000 | 0.640 | 0.03000 | 0.660 | 0.03000 |
| 3.470 | 0.02000 | 16.970 | 0.00091 | 28.070 | 0.00000 | 100.000 | 0.00036 |
| 0.710 | 0.03000 | 0.570 | 0.00012 | 0.580 | 0.01000 | 0.650 | 0.00321 |
| 12.410 | 0.00000 | 15.340 | 0.00000 | 4.170 | 0.00000 | 9.100 | 0.00000 |
| 3.260 | 0.00001 | 3.210 | 0.00002 | 3.260 | 0.00000 | 3.770 | 0.00449 |
| 2.820 | 0.00000 | 3.130 | 0.00009 | 3.110 | 0.00000 | 3.860 | 0.00410 |
| 0.480 | 0.03000 | 0.590 | 0.03000 | 0.440 | 0.01000 | 0.590 | 0.00071 |
| 1.640 | 0.00001 | 2.050 | 0.00001 | 2.270 | 0.02000 | 2.120 | 0.00001 |
| 2.510 | 0.00000 | 3.920 | 0.00000 | 2.320 | 0.00260 | 3.120 | 0.00965 |
| 2.580 | 0.00000 | 4.610 | 0.00000 | 2.930 | 0.00000 | 3.260 | 0.00000 |
| 1.690 | 0.03000 | 2.780 | 0.00000 | 3.380 | 0.00000 | 2.900 | 0.01000 |
| 0.250 | 0.00093 | 0.440 | 0.00028 | 0.170 | 0.00000 | 0.610 | 0.00282 |
| 0.230 | 0.00009 | 0.390 | 0.00100 | 0.110 | 0.00000 | 0.370 | 0.02000 |
| 0.200 | 0.00028 | 0.460 | 0.00007 | 0.470 | 0.04000 | 0.610 | 0.01000 |
| 0.190 | 0.00050 | 0.490 | 0.00386 | 0.210 | 0.00100 | 0.480 | 0.04000 |
| 0.540 | 0.00006 | 0.570 | 0.00048 | 0.440 | 0.00000 | 0.580 | 0.02000 |
| 3.040 | 0.00000 | 2.600 | 0.00000 | 2.070 | 0.00240 | 2.350 | 0.00012 |
| 0.370 | 0.00013 | 0.660 | 0.00160 | 0.600 | 0.00190 | 0.440 | 0.04000 |
| 0.290 | 0.00026 | 0.300 | 0.00000 | 0.430 | 0.02000 | 0.320 | 0.01000 |
| 0.500 | 0.00000 | 0.600 | 0.01000 | 0.580 | 0.00030 | 0.440 | 0.04000 |
| 0.490 | 0.00354 | 0.580 | 0.00048 | 0.250 | 0.03000 | 0.010 | 0.02000 |
| 1.990 | 0.00545 | 3.840 | 0.00000 | 3.110 | 0.00001 | 3.650 | 0.00651 |
| 3.490 | 0.00000 | 5.830 | 0.00000 | 3.980 | 0.00004 | 4.820 | 0.00193 |
| 0.320 | 0.00002 | 0.250 | 0.00000 | 0.270 | 0.00000 | 0.370 | 0.02000 |
| 2.110 | 0.00001 | 2.850 | 0.00038 | 3.980 | 0.00020 | 3.260 | 0.01000 |
| 1.970 | 0.00120 | 2.240 | 0.00028 | 3.560 | 0.00040 | 2.660 | 0.03000 |
| 1.970 | 0.00001 | 2.090 | 0.00002 | 1.660 | 0.00020 | 1.520 | 0.00269 |
| 2.770 | 0.00000 | 2.660 | 0.00000 | 1.690 | 0.00900 | 2.390 | 0.00000 |
| 1.800 | 0.00090 | 2.850 | 0.00000 | 2.170 | 0.00230 | 2.800 | 0.00000 |
| 0.510 | 0.01000 | 0.430 | 0.00001 | 0.250 | 0.00000 | 0.510 | 0.00000 |
| 0.440 | 0.00008 | 0.380 | 0.00002 | 0.300 | 0.00003 | 0.550 | 0.00061 |
| 2.230 | 0.02000 | 4.640 | 0.00000 | 2.580 | 0.00000 | 2.550 | 0.03000 |
| 2.580 | 0.00000 | 5.770 | 0.00000 | 3.110 | 0.00420 | 3.830 | 0.00426 |
| 0.560 | 0.00251 | 0.520 | 0.00007 | 0.510 | 0.02000 | 0.630 | 0.01000 |
| 3.010 | 0.00011 | 11.980 | 0.00000 | 2.150 | 0.00280 | 2.460 | 0.03000 |
| 2.250 | 0.00217 | 18.450 | 0.00000 | 2.410 | 0.00030 | 4.080 | 0.00398 |
| 0.200 | 0.00003 | 0.360 | 0.00000 | 0.380 | 0.00000 | 0.250 | 0.00386 |
| 2.280 | 0.04000 | 1.560 | 0.00524 | 1.820 | 0.00004 | 2.330 | 0.00134 |
| 2.350 | 0.00000 | 2.090 | 0.00105 | 1.810 | 0.04000 | 2.610 | 0.00000 |
| 2.990 | 0.00010 | 3.210 | 0.00000 | 2.560 | 0.00000 | 3.240 | 0.00870 |
| 2.890 | 0.00000 | 2.250 | 0.00002 | 1.920 | 0.00710 | 2.930 | 0.01000 |
| 0.530 | 0.00002 | 0.640 | 0.02000 | 0.430 | 0.00004 | 0.480 | 0.00000 |
| 2.120 | 0.00044 | 2.150 | 0.00000 | 2.360 | 0.00590 | 2.700 | 0.00036 |
| 0.190 | 0.00000 | 0.170 | 0.00000 | 0.240 | 0.00040 | 0.440 | 0.04000 |
| 0.310 | 0.00000 | 0.440 | 0.00002 | 0.440 | 0.00910 | 0.530 | 0.00194 |
| 2.570 | 0.02000 | 3.140 | 0.00000 | 3.220 | 0.00000 | 2.600 | 0.00000 |
| 0.250 | 0.00384 | 0.350 | 0.00012 | 0.310 | 0.00910 | 0.320 | 0.02000 |
| 1.470 | 0.02000 | 3.770 | 0.00000 | 2.750 | 0.00000 | 3.580 | 0.00000 |
| 0.420 | 0.00000 | 0.560 | 0.00037 | 0.440 | 0.00310 | 0.570 | 0.04000 |
| 0.680 | 0.03000 | 0.550 | 0.00497 | 0.480 | 0.00100 | 0.430 | 0.04000 |
| 2.600 | 0.00000 | 3.180 | 0.00000 | 4.990 | 0.00000 | 3.770 | 0.00526 |
| 0.700 | 0.02000 | 0.440 | 0.00000 | 0.520 | 0.00010 | 0.500 | 0.00251 |
| 0.690 | 0.00446 | 0.430 | 0.00000 | 0.500 | 0.00003 | 0.400 | 0.03000 |
| 1.600 | 0.00000 | 1.520 | 0.03000 | 1.420 | 0.04000 | 1.610 | 0.02000 |
| 1.820 | 0.00131 | 1.760 | 0.00007 | 2.100 | 0.00020 | 2.750 | 0.02000 |
| 2.620 | 0.00054 | 4.370 | 0.00000 | 3.920 | 0.00170 | 5.450 | 0.00000 |
| 4.790 | 0.00072 | 11.700 | 0.00000 | 19.810 | 0.00000 | 18.320 | 0.00000 |
| 2.560 | 0.00000 | 6.410 | 0.00000 | 4.140 | 0.00000 | 4.720 | 0.00206 |
| 5.160 | 0.00000 | 2.890 | 0.00005 | 4.430 | 0.00000 | 3.420 | 0.00715 |
| 0.470 | 0.00000 | 0.620 | 0.00767 | 0.580 | 0.00020 | 0.540 | 0.00259 |
| 0.560 | 0.00000 | 0.680 | 0.02000 | 0.660 | 0.00300 | 0.530 | 0.00284 |
| 0.830 | 0.01000 | 0.510 | 0.00002 | 0.440 | 0.00000 | 0.390 | 0.02000 |
| 0.650 | 0.00004 | 0.340 | 0.00000 | 0.390 | 0.00220 | 0.290 | 0.00714 |
| 0.730 | 0.03000 | 0.450 | 0.00013 | 0.330 | 0.00000 | 0.400 | 0.03000 |
| 0.550 | 0.00477 | 0.570 | 0.00050 | 0.560 | 0.00510 | 0.470 | 0.00048 |
| 0.680 | 0.04000 | 0.660 | 0.02000 | 0.650 | 0.01000 | 0.510 | 0.02000 |
| 1.410 | 0.02000 | 1.880 | 0.00149 | 2.200 | 0.00003 | 2.340 | 0.04000 |
| 2.060 | 0.00028 | 1.760 | 0.00037 | 1.720 | 0.00850 | 1.830 | 0.00001 |
| 0.330 | 0.00002 | 0.330 | 0.00000 | 0.190 | 0.00000 | 0.430 | 0.04000 |
| 0.350 | 0.00120 | 0.400 | 0.00000 | 0.260 | 0.00000 | 0.420 | 0.00001 |
| 0.170 | 0.00000 | 0.250 | 0.00000 | 0.220 | 0.00000 | 0.290 | 0.00655 |
| 0.170 | 0.00000 | 0.260 | 0.00000 | 0.250 | 0.00000 | 0.270 | 0.00487 |
| 0.310 | 0.00000 | 0.240 | 0.00000 | 0.280 | 0.00000 | 0.300 | 0.00824 |
| 0.070 | 0.00000 | 0.130 | 0.00000 | 0.160 | 0.00000 | 0.210 | 0.00000 |
| 3.240 | 0.00033 | 5.150 | 0.00000 | 3.440 | 0.00000 | 3.550 | 0.00578 |
| 0.530 | 0.00000 | 0.510 | 0.00343 | 0.550 | 0.00080 | 0.510 | 0.00003 |
| 0.410 | 0.00030 | 0.550 | 0.00007 | 0.460 | 0.00280 | 0.600 | 0.00937 |
| 0.310 | 0.00001 | 0.370 | 0.00001 | 0.250 | 0.00002 | 0.280 | 0.00000 |
| 0.440 | 0.00010 | 0.730 | 0.03000 | 0.510 | 0.02000 | 0.720 | 0.03000 |
| 0.670 | 0.03000 | 0.600 | 0.00134 | 0.440 | 0.00000 | 0.730 | 0.02000 |
| 2.510 | 0.00000 | 1.600 | 0.00613 | 2.200 | 0.00090 | 2.250 | 0.04000 |
| 3.330 | 0.00000 | 2.070 | 0.00159 | 2.350 | 0.00005 | 3.380 | 0.00000 |
| 2.590 | 0.00002 | 2.500 | 0.00000 | 2.850 | 0.00160 | 2.840 | 0.00003 |
| 2.920 | 0.00000 | 2.950 | 0.00000 | 2.480 | 0.00110 | 3.710 | 0.00499 |
| 2.740 | 0.00000 | 1.930 | 0.00104 | 2.080 | 0.00100 | 2.400 | 0.03000 |
| 2.630 | 0.00000 | 1.990 | 0.00051 | 2.230 | 0.00000 | 2.470 | 0.04000 |
| 2.810 | 0.00000 | 1.880 | 0.00018 | 2.210 | 0.00008 | 2.110 | 0.00020 |
| 3.190 | 0.00000 | 1.870 | 0.00134 | 2.570 | 0.00000 | 2.250 | 0.04000 |
| 2.590 | 0.00000 | 1.610 | 0.01000 | 2.380 | 0.00002 | 2.270 | 0.04000 |
| 2.630 | 0.00008 | 1.600 | 0.00341 | 2.170 | 0.00080 | 2.450 | 0.00000 |
| 2.260 | 0.00001 | 1.680 | 0.00214 | 1.990 | 0.00320 | 1.860 | 0.00465 |
| 0.600 | 0.00607 | 0.400 | 0.00000 | 0.540 | 0.00005 | 0.420 | 0.03000 |
| 0.490 | 0.00005 | 0.450 | 0.00002 | 0.510 | 0.00360 | 0.500 | 0.00000 |
| 1.560 | 0.00000 | 2.090 | 0.00023 | 2.220 | 0.00000 | 2.050 | 0.00354 |
| 2.220 | 0.00000 | 2.050 | 0.00133 | 1.490 | 0.00980 | 1.530 | 0.04000 |
| 2.750 | 0.00000 | 2.550 | 0.00000 | 3.460 | 0.00020 | 3.660 | 0.00716 |
| 0.380 | 0.00000 | 0.330 | 0.00002 | 0.460 | 0.00001 | 0.350 | 0.01000 |
| 1.860 | 0.02000 | 2.010 | 0.00002 | 4.180 | 0.00007 | 3.310 | 0.01000 |
| 1.640 | 0.00002 | 2.090 | 0.00007 | 2.400 | 0.00000 | 3.270 | 0.01000 |
| 4.040 | 0.00000 | 6.190 | 0.00000 | 2.580 | 0.00005 | 3.030 | 0.01000 |
| 6.580 | 0.00000 | 7.860 | 0.00000 | 2.870 | 0.00240 | 3.040 | 0.01000 |
| 7.770 | 0.00000 | 7.680 | 0.00000 | 2.670 | 0.00050 | 3.550 | 0.00570 |
| 2.640 | 0.00245 | 2.140 | 0.04000 | 2.300 | 0.04000 | 3.900 | 0.00000 |
| 0.570 | 0.00001 | 0.320 | 0.00112 | 0.400 | 0.00230 | 0.390 | 0.02000 |
| 0.720 | 0.02000 | 0.400 | 0.00000 | 0.470 | 0.03000 | 0.390 | 0.03000 |
| 2.760 | 0.00040 | 2.410 | 0.00004 | 2.420 | 0.00920 | 3.050 | 0.01000 |
| 1.980 | 0.00005 | 2.440 | 0.00001 | 2.710 | 0.00140 | 3.000 | 0.01000 |
| 2.500 | 0.00199 | 3.730 | 0.00000 | 5.380 | 0.00000 | 5.920 | 0.00118 |
| 1.540 | 0.03000 | 3.210 | 0.00027 | 7.600 | 0.00960 | 4.220 | 0.00538 |
| 1.990 | 0.00120 | 2.300 | 0.00000 | 2.530 | 0.00270 | 3.180 | 0.01000 |
| 4.020 | 0.00384 | 5.790 | 0.00585 | 35.490 | 0.00001 | 100.000 | 0.00000 |
| 0.480 | 0.00000 | 0.270 | 0.00000 | 0.190 | 0.00040 | 0.310 | 0.02000 |
| 0.480 | 0.00000 | 0.270 | 0.00000 | 0.170 | 0.00000 | 0.290 | 0.02000 |
| 0.680 | 0.00127 | 0.610 | 0.00313 | 0.360 | 0.00000 | 0.440 | 0.04000 |
| 0.590 | 0.00000 | 0.720 | 0.04000 | 0.620 | 0.00500 | 0.420 | 0.03000 |
| 1.570 | 0.00245 | 3.310 | 0.00000 | 4.700 | 0.00020 | 3.440 | 0.00000 |
| 3.910 | 0.00000 | 2.210 | 0.00221 | 2.410 | 0.03000 | 2.850 | 0.01000 |
| 1.940 | 0.00008 | 2.510 | 0.00001 | 2.250 | 0.00007 | 2.580 | 0.03000 |
| 2.380 | 0.02000 | 5.490 | 0.00000 | 7.970 | 0.00000 | 6.930 | 0.00097 |
| 2.040 | 0.00000 | 1.750 | 0.00419 | 1.590 | 0.00760 | 1.840 | 0.02000 |
| 0.250 | 0.00000 | 0.270 | 0.00000 | 0.620 | 0.03000 | 0.320 | 0.01000 |
| 0.440 | 0.00001 | 0.620 | 0.01000 | 0.690 | 0.00790 | 0.540 | 0.00005 |
| 2.850 | 0.00000 | 4.090 | 0.00000 | 1.670 | 0.00120 | 2.030 | 0.00772 |
| 3.340 | 0.00000 | 3.490 | 0.00000 | 1.890 | 0.00004 | 2.680 | 0.00000 |
| 3.260 | 0.00000 | 4.750 | 0.00000 | 1.770 | 0.00160 | 2.900 | 0.00000 |
| 1.330 | 0.03000 | 1.840 | 0.00030 | 2.160 | 0.00130 | 2.990 | 0.04000 |
| 2.000 | 0.02000 | 1.680 | 0.00087 | 2.130 | 0.00650 | 2.000 | 0.00448 |
| 1.470 | 0.00979 | 1.650 | 0.02000 | 2.350 | 0.02000 | 2.890 | 0.01000 |
| 0.400 | 0.00000 | 0.670 | 0.00673 | 0.590 | 0.03000 | 0.570 | 0.00336 |

### Example 2

### Foxo Transcription Factors Induce the Atrophy-Related Ubiquitin Ligase Atrogin-1 and Cause Skeletal Muscle Atrophy

Skeletal muscle atrophy is a debilitating, poorly understood response to fasting, disuse and many systemic diseases. In atrophying muscles, the ubiquitin ligase, atrogin-1 (MAFbx), is induced 8-40 fold, and this response is necessary for rapid wasting. Here we show using in vitro models of atrophy, that there is a decrease in the PI3K/AKT pathway, activation of the forkhead (Foxo) family, and induction of atrogin-1. IGF-1 treatment or AKT overexpression cause Foxo inhibition and block atrogin-1 expression. Moreover, constitutively active Foxo3 alone causes atrogin-1 expression and dramatic atrophy of both cultured myotubes and fibers in adult mouse muscles. Mutating the several potential Foxo binding sites in the atrogin-1 promoter abolishes atrogin-1 induction by Foxo3 in adult muscles. Furthermore, when Foxo activation is blocked by a dominant negative construct in culture or RNAi in mice, the induction of atrogin-1 by starvation and reduction in myotube size by glucocorticoids are prevented. Thus, forkhead factor (s) play a critical role in the development of muscle atrophy and inhibition of Foxo function could be a novel approach to combat various forms of muscle wasting.

### INTRODUCTION

Muscle atrophy occurs systemically in fasting and a wide range of diseases, including diabetes mellitus, cancer, AIDS, sepsis, and hyperadrenalcortisolism, and in specific muscles following denervation or disuse (Booth and Criswell, 1997; Lecker et al., 1999). The molecular mechanisms that underlie this process are just beginning to be uncovered. In these diverse types of atrophy, the muscles all show increased rates of protein degradation primarily through activation of the ubiquitin-proteasome pathway (Attaix et al., 2001; Jagoe and Goldberg, 2001; Solomon et al., 1998) and a common series of transcriptional adaptations that together constitute an “atrophy program” (Jagoe et al., 2002; Lecker et al., 2004). Among the genes induced in these muscles are polyubiquitin and certain proteasome subunits that support the enhanced rates of proteolysis by the ubiquitin (Ub)-proteasome pathway. The enzyme that is induced most dramatically in these atrophying muscles is the muscle-specific Ub-ligase, atrogin-1 (MAFbx) (Bodine et al., 2001a; Gomes et al., 2001). mRNA for atrogin-1 increases 8-40 fold in all types of atrophy studied, and this increase precedes the onset of muscle weight loss (Gomes et al., 2001). Moreover, knockout animals lacking atrogin-1 show a reduced rate of muscle atrophy after denervation (Bodine et al., 2001a). Consequently, inhibiting atrogin-1 activity or its induction are attractive possible pharmacological approaches to retard muscle atrophy.

Since various types of muscle atrophy share a common set of transcriptional adaptations, it seems likely that the diverse stimuli that lead to atrophy act through common signaling mechanisms and influence the same transcription factor (s). A variety of endocrine changes are known to activate protein degradation and lead to systemic muscle wasting (Kettelhut et al., 1988). Low levels of insulin and probably low IGF-1 levels, together with elevated levels of glucocorticoids, trigger the loss of muscle protein after food deprivation and in diabetes (Mitch et al., 1999; Wing and Goldberg, 1993). Insulin resistance is a characteristic feature of systemic diseases such as cancer, uremia and sepsis that also appears to contribute to muscle wasting (Zierath et al., 2000). In large doses, glucocorticoids by themselves cause muscle wasting (Kayali et al., 1987), and these steroids are necessary for the catabolic response in fasting, diabetes, and sepsis (Hasselgren, 1999; Tiao et al., 1996). In muscle, these steroids reduce protein synthesis and enhance proteolysis (Hasselgren, 1999). It is therefore important to define the signal transduction pathways by which glucocorticoids and low insulin trigger loss of muscle protein.

Several recent findings suggest that decreased activity of the IGF-1/PI3K/AKT signaling pathway can lead to muscle atrophy (Bodine et al., 2001b). Inhibition of PI3K reduces the mean size of myotubes in culture. Also, inhibition of mTOR, a target of AKT, by rapamycin can prevent growth of muscle fiber during regeneration in vivo (Pallafacchina et al., 2002), while expression of a dominant-negative AKT causes a decrease in the size of cultured myotubes and prevents myofiber growth during regeneration in mice (Pallafacchina et al., 2002; Rommel et al., 2001). On the other hand, activation of AKT in rat muscle can prevent atrophy induced by denervation (Pallafacchina et al., 2002; Rommel et al., 2001). Much of the growth promotion by IGF-1, insulin, and activated AKT is through a general increase in protein synthesis (see below), and decreased AKT activity probably causes the decrease in protein translation seen in many types of muscle atrophy. However, in related studies (Sacheck et al., manuscript submitted), we have obtained pharmacological evidence that inhibition of PI3K can activate protein degradation in muscle and stimulate expression of atrogin-1 in mouse myotubes, and that IGF-1 and insulin suppress these processes.

To elucidate the intracellular signaling events that lead to muscle wasting, we initially used as a simple in vitro model of muscle atrophy, myotubes treated with glucocorticoids. As demonstrated elsewhere (Sacheck et al., manuscript submitted), dexamethasone increases overall rates of proteolysis and especially the degradation of myofibrillar proteins in C2C12 myotubes (as it does in vivo), and these responses can be suppressed by IGF-1 or insulin. Furthermore, the content of atrogin-1 mRNA correlates tightly with overall rates of protein degradation in these cells. These experiments strongly suggest that the IGF-1/PI3K/AKT pathway suppresses protein degradation, expression of atrogin-1 and other atrophy-related genes, and that these effects together with the complementary stimulation of protein synthesis, retard atrophy and favor muscle growth.

IGF-1 is essential for postnatal growth of muscle as well as for the maintenance of adult muscle size. Circulating IGF-1 mediates the anabolic actions of growth hormone and is produced locally in muscle during exercise (DeVol et al., 1990). The binding of IGF-1 or insulin to their receptors activates two major signal transduction pathways: the Ras-Raf-MEK-ERK pathway and the PI3K/AKT pathway. In adult skeletal muscle, the Ras-Raf-MEK-ERK pathway affects fiber type composition, but has no effect on fiber size (Murgia et al., 2000; Serrano et al., 2001). However, activation of the PI3K/AKT pathway induces skeletal muscle hypertrophy by stimulating translation via the AKT/GSK and AKT/mTOR pathways (Bodine et al., 2001b). AKT causes phosphorylation and inhibition of GSK3β, which leads to increased protein synthesis in muscle by activation of the key translation initiation factor, eIF2B. In addition, AKT-mediated phosphorylation of mTOR increases its activity, which in turn activates S6K and inactivates 4E-BP1, an inhibitor of translation initiation. Thus, overexpression of AKT or S6K promotes protein synthesis and induces myotube hypertrophy, and in mice, activation of the AKT/mTOR pathway also leads to larger fiber size (Bodine et al., 2001b; Pallafacchina et al., 2002; Rommel et al., 2001). In addition to its endocrine regulation through AKT, mTOR activity is also controlled by the availability of amino acids and glucose (Hara et al., 2002; Kim et al., 2003). High levels of amino acids cause phosphorylation of mTOR targets, while low levels of nutrients lead to dephosphorylation of S6K. In this way, mTOR can integrate signals from both the IGF-1/PI3K/AKT pathway as well as information about the cell's nutritional status. During fasting in vivo, there is both reduced production of insulin and IGF-1 together with reduced uptake of nutrients by muscles and blockage of nutrient transport into muscles, all of which would be expected to suppress mTOR activity.

One downstream target of the PI3K/AKT pathway that could mediate the IGF-1 effects on atrogin-1 and muscle atrophy is the winged helix or Forkhead box (Foxo) class of transcription factors. The Foxo family of transcription factors represents a subfamily within the larger group of forkhead transcription factors, and in mammals consists of three members, Foxo1 (also known as FKHR), Foxo3 (also called FHKRL1, FOXO3A in humans), and Foxo4 (also called AFX) (Tran et al., 2003). AKT blocks the function of all these Foxo family members by phosphorylation of three conserved residues, leading to their sequestration in the cytoplasm where they are unable to act on target promoters (Brunet et al., 1999). On the other hand, dephosphorylation of Foxo factors leads to entry into the nucleus and generally results in growth-suppression or apoptosis (Ramaswamy et al., 2002). In addition, the expression of Foxo genes is also tightly regulated. Fasting and glucocorticoid treatment induce the expression of Foxo factors in mouse liver and skeletal muscle, while refeeding suppresses Foxo transcription (Furuyama et al., 2003; Imae et al., 2003; Kamei et al., 2003). In addition, we have found recently that Foxo1 is one of the atrophy-related genes (‘atrogenes’)¹ that is induced in muscles atrophying due to diverse diseases (Lecker et al., 2004). Finally, Foxo factors are necessary for the development of insulin resistance in type II diabetes in liver, pancreas and adipose tissue (Nakae et al., 2002; Nakae et al., 2003; Puigserver et al., 2003).
¹These atrophy-related genes had formerly been termed ‘atrogins’, but because of the potential confusion with the ubiquitin-ligase, atrogin-1, we shall hereafter refer to them as ‘atrogenes’.

In this study, we have used two simple in vitro models of muscle atrophy cell starvation and dexamethasone treatment to identify the downstream targets of the IGF1/PI3K/AKT pathway that are important for the induction of the key Ub-protein ligase, atrogin-1, and to the development of muscle wasting. We demonstrate here that IGF-1 acts through AKT and Foxo to suppress atrogin-1 expression, and that the mTOR/S6K, GSK and NFκB pathways (previously proposed to regulate the atrophy process) are not involved in regulating atrogin-1 expression. We show that Foxo3 is able to strongly induce atrogin-1 expression by acting directly on the atrogin-1 promoter, and that Foxo3 expression by itself leads to a reduction in myotube size. Additional studies in mice show that Foxo factors also play a similar role in adult muscle. To study the effect of Foxo factors in adult muscle, we have used a novel direct transfection technique to show that expression of active Foxo3 leads to marked upregulation of atrogin-1 mRNA, transcription of the atrogin-1 promoter, and surprisingly, overall reduction of muscle fiber size. These observations indicate a new and unexpected pathway for development of atrophy—decrease in AKT activity leading to activation of Foxo family members. Moreover, discovery of the key role of Foxo in triggering the atrophy program should lay the basis not only for further understanding of the mechanisms of muscle wasting in diverse diseases, but also for developing novel therapies for these debilitating conditions.

### METHODS

Antibodies and Reagents

Anti-phospho-AKT (Ser473), anti-phospho-mTOR (Ser2448), anti-phospho-p70S6K (Thr389), anti-phospho-Foxo1 (Ser 256), anti-phospho-Foxo4 (Ser 193), anti-AKT, anti-p70S6K and anti-Foxo1 were purchased from Cell Signaling. Anti-phospho-Foxo3 (Thr32) and the FKHR (Foxo1)-GST fusion protein were from Upstate. All primary antibodies were rabbit potyclonal antibodies. HRP-conjugated goat anti-rabbit antibody was from Promega.

Cell Culture, Adenoviral Infection and Myotube Analysis

C2C12 mouse myoblasts (ATCC) were cultured in DMEM 10% FCS (ATCC) until the cells reached confluence. At that time, the medium was replaced with DMEM 2% horse serum (HS) (ATCC) (“differentiation medium”) and incubated for 4 days to induce myotube formation before proceeding with experiments. Pharmacological agents were variously used at the following final concentrations: dexamethasone (1 μM; Sigma), IGF-1 (10 ng/ml; Sigma), LiCl (10 mM; Sigma), LY294002 (10 μM, from 10 mM stock in DMSO, Calbiochem). For infection, myotubes were incubated with adenovirus at a multiplicity of infection (MOI) of 250 in differentiation medium for 18 h, and then medium was replaced. Under these conditions, infection efficiency was greater than 90%. Typical experiments for RNA and protein analysis were performed in 6-well plates. Myotube diameter was quantified as follows: five different fields at 100× magnification were chosen randomly and myotubes were measured using IMAGE software (Scion, Frederick, Md.) as previously performed (Rommel et al., 2001). All data are expressed as the mean of five measurements taken along the length of the myotube±SEM. Comparisons were made using the student's t test, with p<0.05 being considered statistically significant.

Adenoviral Vectors

The dominant-negative AKT (d.n.AKT), the constitutively active AKT (c.a.AKT), the constitutively active GSK3 (c.a.GSK), the dominant-negative GSK3 (d.n.GSK), the constitutively active NIK (c.a.NIK), the constitutively active IκB (c.a.IκB), the wild type human Foxo3 (FOXO3A), the constitutively active human Foxo3 (c.a.FOX3A), the dominant-negative human Foxo3 (d.n.FOXO3A), the control GFP and the control (βgal have been described previously (Kim et al., 2002; Skurk et al., 2003).

RNA Extraction and Northern Blot Analysis

After incubation, myotubes were washed in PBS and total RNA was extracted using TRizo1 reagent (Invitrogen) according to the manufacturer's specifications. Northern blotting was performed as described elsewhere (Gomes et al., 2001). Briefly, total RNA was separated on formaldehyde-agarose gels, transferred to Zeta-Probe membrane (Bio-Rad) and UV-crosslinked. Membranes were hybridized with a full length mouse atrogin-1 probe, at 65 ° C., in Church buffer (Church and Gilbert, 1984), and analyzed by using a Fuji Phosphorimager. Blots were stripped and reprobed with a mouse GADPH probe (Ambion) to ensure equal-loading and gel transfer. The relative amounts of the bands were quantified by densitometry.

Protein Extraction and Western Blotting

Myotube protein was also extracted with TRizo1 reagent according to the manufacturer's instructions. Protein concentration was determined by BCA assay (Pierce). SDS-PAGE was performed on 4-12% gradient gels (Novex, Invitrogen) loading 20 μg protein/lane. Westerns were performed as previously (Gomes et al., 2001) and were visualized with ECL detection reagents (Amersham). Blots were stripped using Restore Western Blotting Stripping Reagent (Pierce) according to the manufacturer's instructions and reprobed if necessary. The relative amounts of the bands were quantified by densitometry using ImageJ software.

Atrogin-1 Promoter Cloning

3.5 kb of genomic sequence immediately 5′ of the atrogin-1 ATG was amplified from mouse J1 embryonic stem cell DNA using the Genomic-GC PCR amplification kit (BD Biosciences). Unique KpnI and Bg1II sites were incorporated at the 5′ and 3′ ends of the sequence, respectively, to simplify directional cloning into KpnI and Bg1II sites in the reporter plasmid, pGL3-basic (Promega). Primers: forward: 5′-GGGGTACCCTTCTCCAGGCCAGTAGGTG-3′ (SEQ ID NO:1), reverse: 5′-GGAAGATCTTGGTACAGAGCGCGGACGCG-3′ (SEQ ID NO:2). Smaller fragments of the atrogin-1 promoter were generated by PCR using cloned Pfu polymerase (Stratagene) and the 3.5 kb promoter construct as a template. 1.0 kb fragment: forward: 5′-GGTACCGCCAGGCGCCTCGACGCC-3′ (SEQ ID NO:3), 0.4 kb fragment: forward: 5′-GGGGTACCGGCGAGCCTATAAACAAAGCC-3′ (SEQ ID NO:4). The same reverse primer (above) was used in each case. The KpnI/BglII digested PCR product was subsequently ligated into KpnIBglII digested pGL3-basic. The putative TATAA box is at −360 and the transcriptional start site is at −263 from the ATG based on information from GenBank. Introduction of mutations in the two putative Foxo binding sites in the 0.4 kb fragment (Foxo₁ at +2 and Foxo₂ at −94 relative to the transcription start site) were generated by PCR using the QuickChange technique (Stratagene) according to the manufacturer's instructions with the following primers (mutations are underlined): Foxo₁, 5,-GGGCAGCGGCCCGGGTACCGTACAGTGCTCGGGCAG-3′ (SEQ ID NO:5) and 5′CTGCCCGAGCACTGTACGGTACCCGGGCCGCTGCCC-3′ (SEQ ID NO:6). Foxo₂ 5′-TATCGATAGGTACCGGCTAGCCTATAAGCTCAGCCACGTGGCCTC-3′ (SEQ ID NO:7) and 5′-GAGGCCACGTGGCTGAGCTTATAGGCTAGCCGGTACCTATCGATA-3′ (SEQ ID NO: 8).

Transient Transfections and Luciferase Assays

Myoblasts were transfected using Lipofectamine2000 (Invitrogen) according to the manufacturer's instructions. Briefly, 2×10⁴ C2C12 cells were seeded into individual wells of 12-wells plates 24 h prior to transfection with either the 3.5 kb atrogin-1- or 1.0 kb atrogin-1-luciferase constructs and pRL-TK (Promega) (1 μg total DNA/well, 1:1 reporter:pRL-TK). When the cells reached confluence, the medium was shifted to differentiation medium to induce myotube formation. After 4 days, myotubes were infected as described above. 36 h later, myotubes were lysed and analyzed using the Dual-Luciferase reporter assay system (Promega) as directed by the manufacturer. Firefly luciferase activity was divided by Renilla luciferase activity to control for transfection efficiency. Luminescence measurements in muscles transfected with reporter constructs were performed similarly, except that the harvested muscles were quick frozen and powdered in liquid nitrogen before the addition of the lysis buffer (Serrano et al., 2001).

Electrophoretic Mobility Shift Assay

Double stranded oligonucleotides were labeled by T4 Polynucleotide kinase and γ-[³²P] dATP (Promega). The following pairs of oligonucleotides were used (Forkhead sites are underlined): IGFBP1: 5′-CTAGCAAGCAAAACAAACTTATTTTGAACACGGGG-3′ (SEQ ID NO:9) and 5′-CCCCGTGTTCAAAATAAGTTTGTTTTGCTTGCTAG (SEQ ID NO:10); SP 1:5′-ATTCGATCGGGGCGGGGCGAG-3′ (SEQ ID NO:11) and 5′CTCGCCCCGCCCCGATCGTAA-3′ (SEQ ID NO:12); AT_{Foxo 1}: 5′-GGGATAAATACT-GTGCTCGGGCAG-3′ (SEQ ID NO:13) and 5′-CTGCCCGAGCACAGTATTTATCCC-3′ (SEQ ID NO:14). AT_{Foxo 1mut}: 5′-GGGATCACTACTGTGCTCGGGCAG-3′ (SEQ ID NO:15) and 5′ CTGCCCGAGCACAGTAGTGATCCC-3′ (SEQ ID NO:16). Reactions using the Gel Shift Assay System (Promega) contained the cold competitor (100-fold molar excess over labeled oligonucleotide) and 1 μl of purified FoxoGST (Upstate), and were incubated at room temperature for 10 min. The samples were then mixed with ³²P-labeled oligonucleotides for 20 min and loaded on Novex 6% DNA retardation gels (Invitrogen). After electrophoresis the gels were dried and analyzed using a Fuji Phosphorimager system.

Adult Mouse Skeletal Muscle Transfection

Adult female CD1 mice (28-30 g) were used in all experiments. Tibialis anterior muscles (Kamei et al., 2003) were transfected as described previously (Murgia et al., 2000; Serrano et al., 2001). Briefly the muscle was isolated through a small hindlimb incision, and 25 μg of plasmid DNA was injected along the muscle length. In reporter experiments 10 μg of the expression vector with 10 μg of the 3.5 kb atrogin-1-firefly luciferase reporter construct and 5 μg of pRL-TK vectors were co-injected. Immediately after the plasmid injection, electric pulses were applied by two stainless steel spatula electrodes placed on each side of the isolated muscle belly (50Volts/cm, 5 pulses, 200 ms intervals). Muscles were analyzed 4 or 8 days later. Morphologically, the muscles appear normal during the course of these experiments. No gross or microscopic evidence for necrosis or inflammation as a result of the transfection procedure is noted. The following constructs, all containing an HA tag were used: a constitutively active AKT, a constitutive active Foxo3, and a wild type Foxo3. All these vectors have been previously described (Brunet et al., 1999; Pallafacchina et al., 2002). A reporter containing six forkhead binding sites (DAF16 binding elements, DBE) (Furuyama et al., 2000) was generated by ligation of the following oligonucleotides to NheI-XhoI double digested pGL3-basic vector. DBE oligonucleotides:

| | |
|---|---|
| 5′ TCGAAAGTAAACAACTATGTAAACAACTATAAGTAAACAACT | (SEQ ID NO:17) |
| ATGTAAACAACTATAAGTAAACAACTATGTAAACAAGATC-3′ | |
| and | |
| 5′-CTAGGATCTTGTTTACATAGTTGTTTACTTATAGTTGTTTACATAGTTGTTTAC | (SEQ ID NO:18) |
| TTATAGTTGTTTACATAGTTGTTTACTT-3′. | |

Immunhistochemistry and Fiber Size Measurements

Mouse muscle fibers expressing HA-tagged proteins were stained in cryo-cross sections fixed with 4% paraformaldehyde. Immunohistochemistry with anti-HA polyclonal antibody (Santa Cruz) was as previously described (Pallafacchina et al., 2002). Muscle fiber size was measured in fibers transfected with the Foxo3 mutant and in an equal number of untransfected fibers from the same muscle as described elsewhere (Pallafacchina et al., 2002). Fiber cross-sectional areas were measured using IMAGE software (Scion, Frederick, Md.). All data are expressed as the mean±SEM. Comparison were made by using the student's t test, with p<0.05 being considered statistically significant.

In Situ Hybridization

In situ hybridization was performed as described (Murgia et al., 2000). An ³⁵S-labeled cRNA probe complementary to the atrogin-1 coding region was prepared by in vitro transcription (Roche) using the full length mouse atrogin-1 gene in KS⁺Bluescript as a template.

RNAi in Adult Skeletal Muscle

A target finder and design tool (Ambion) was used to identify target regions in the mouse Foxo1 and 3 and GFP genes amenable to siRNA. Foxo1 and 3: 5′ GGATAAGGGCGACAGCAAC-3′ (SEQ ID NO:19), GFP: 5′-CTGGACTTCCAGAAGAACA-3′ (SEQ ID NO:20). These sequences were incorporated into 64 bp self-annealing oligonucleotides (Brummelkamp et al., 2002), synthesized and cloned into Bg1II-HindIII double-digested pSUPER vector (Brummelkamp et al., 2002). Adult skeletal muscle was cotransfected with 30 μg of the pSUPER vector along with 10 μg of the 3.5 kb atrogin-1-firefly luciferase reporter construct and 5 μg of pRL-TK vectors as above described. 7 days after transfection, the mice were fasted for 24 hr and sacrificed.

### RESULTS

The PI3K/AKT Pathway is Suppressed in Cell Culture Models of Muscle Atrophy

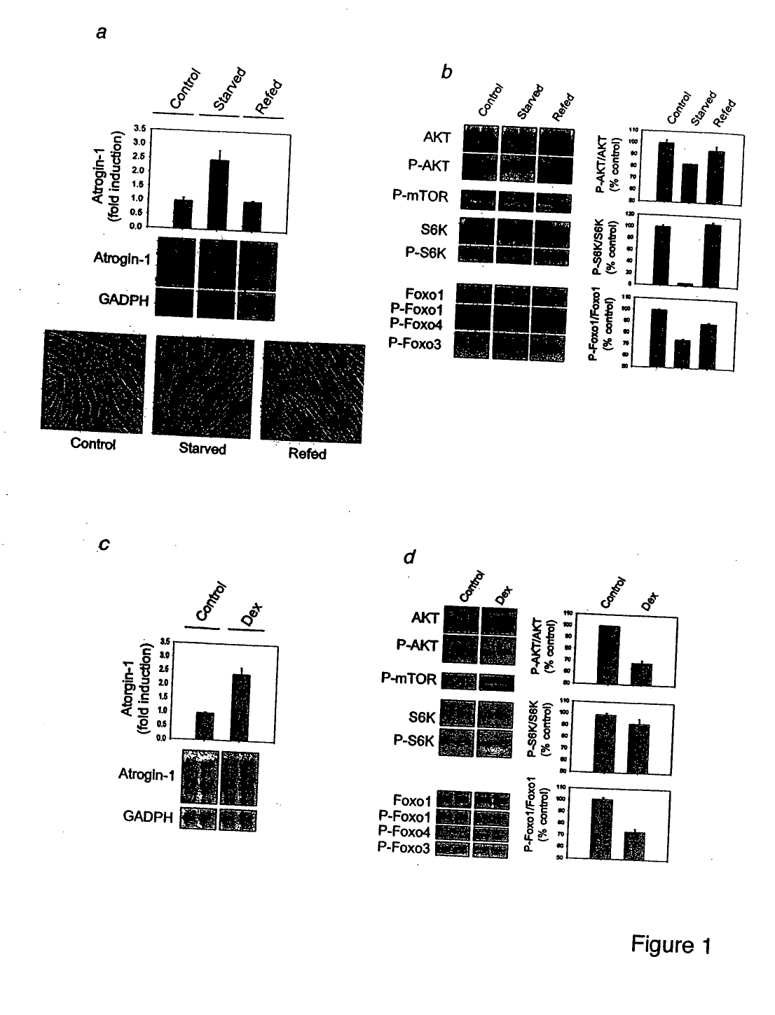

Our initial goal was to identify the signal transduction pathways that activate expression of atrogin-1 in various atrophying muscles (Bodine et al., 2001 a; Gomes et al., 2001) and that suppress its transcription under normal conditions. As a first step, we characterized the changes in atrogin-1 mRNA content and intermediates in the PI3K/AKT pathway in two experimental conditions that we found caused decrease in size of cultured C2C12 cells and mimic features of muscle atrophy in vivo. Because complete food deprivation leads to rapid muscle wasting and an 8-10 fold upregulation of atrogin-1 mRNA (Gomes et al., 2001), we studied the effects of starving cultured myotubes of serum, glucose and amino acids. In addition, similar protocols have been used to study the role of mTOR in other cell types (Peng et al., 2002). After 6 hours, these cells were completely viable but showed a remarkable reduction in myotube diameter (60% decrease, FIG. 1a), and contained 2.5-fold more atrogin-1 mRNA than fed cells (FIG. 1a). These changes were readily reversible. If the cells starved for 6 hrs were re-supplied with serum, amino acids, and glucose, atrogin-1 mRNA decreased back to control levels, and the original cell size was restored within 12 hrs (FIG. 1a).

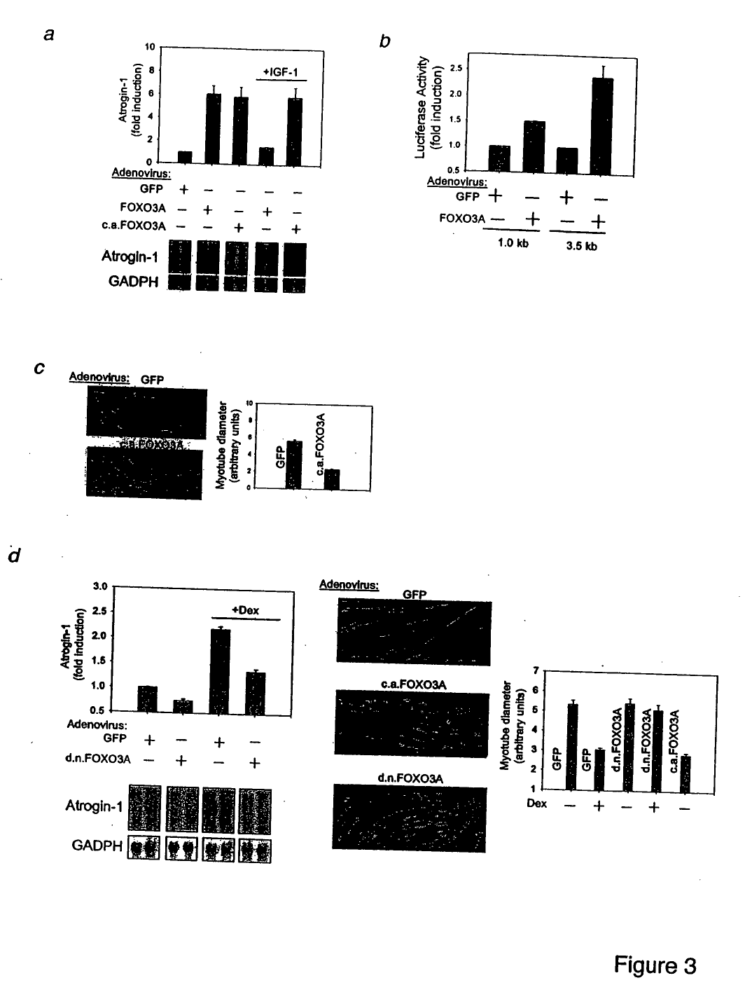

Related studies from this laboratory (Sacheck et al., manuscript submitted) have shown that the glucocorticoid, dexamethasone, induces atrogin-1, stimulates protein breakdown, and causes a loss of protein and RNA content in C2C12 cells (just as it can do in adult mammalian muscles). Accordingly, when we treated cultured myotubes with dexamethasone for 24 hrs, there was a 2-3-fold increase in atrogin-1 mRNA content (FIG. 1c) and a 40% reduction in mean myotube diameter (see FIG. 3d).

Because these responses mimic the major features of atrophy in adult muscles, we attempted to define the changes in the PI3K/AKT/mTOR pathway in these cells, since inhibition of this pathway can induce muscle atrophy in vitro and in vivo (Bodine et al., 2001b), and since by activating this pathway, IGF-1 stimulates cell growth (Rommel et al., 2001). We have specifically investigated the possible involvement of the forkhead transcription factors Foxo1, 3 and 4, which are downstream targets of AKT, because expression of Foxo1 and 3 rise in skeletal muscle during fasting (Furuyama et al., 2000) and other types of atrophy (Lecker et al., 2004). We first tested whether the level of phosphorylation of different components of the PI3K/AKT/Foxo pathway change in response to dexamethasone treatment for 24 hrs or starving myotubes of serum and nutrients for 6 hrs. Both these treatments not only increased atrogin-1 expression but also reduced levels of phosphorylated AKT below levels in control cultures (FIG. 1b, d). Densitometric analysis revealed that the ratio of phospho-AKT to total AKT fell consistently by 20% after nutrient-deprivation and by 30% after dexamethasone treatment.

A reduction in AKT activity (i.e. AKT dephosphorylation) would be expected to lead to decreased phosphorylation of Foxo1, 3 and Foxo4. In fact, the levels of phosphorylated Foxo1 and 3 decreased by at least 30% after starvation and glucocorticoid treatment, and Foxo4 showed a 50% reduction in the starved myotubes. The removal of serum and nutrients also led to an almost complete dephosphorylation of S6K, as would be expected, since S6K is phosphorylated by both AKT and by the nutrient-sensitive mTOR/RAPTOR/GβL complex (Kim et al., 2003). However, it is also possible that the marked dephosphorylation of S6K may partially reflect activation in these cultures of a phosphatase, such as PP2A (Peterson et al., 1999). The re-addition of serum, amino acids and glucose to the starved cells increased the phosphorylation of AKT, Foxo factors, and S6K and decreased atrogin-1 mRNA to control levels. In contrast to the marked dephosphorylation of S6K in the starved cells, the level of phosphorylated S6K decreased only slightly (10%) after dexamethasone treatment. Thus, in these two conditions, the dephosphorylation of Foxo transcription factors (but not of S6K) correlates with atrogin-1 induction. Subsequent experiments therefore tested if the Foxo family might, in fact, transcribe the atrogin-1 gene (see below).

Atrogin-1 Expression is Suppressed by IGF-1 Through the PI3K/AKT Pathway

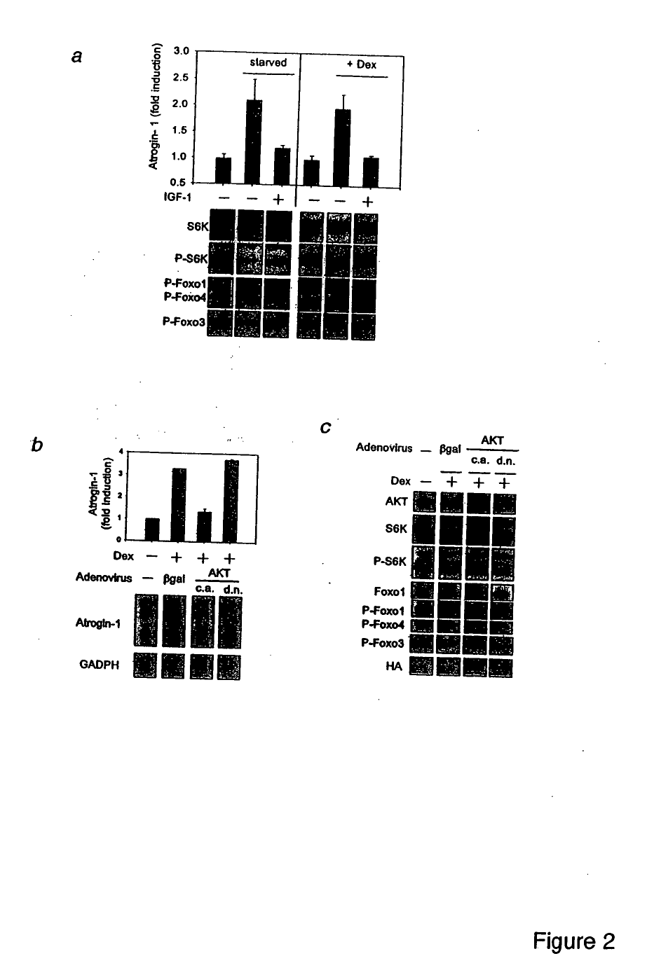

Studies from this laboratory have shown that LY 294002, an inhibitor of PI3K, stimulates atrogin-1 expression (Sacheck et al., manuscript submitted). To test further if the PI3K/AKT pathway suppresses expression of atrogin-1, we measured whether the addition of IGF-1 could prevent dephosphorylation of AKT and Foxo1, 3, and 4 and block the high level of atrogin-1 expression in the starved cells and after dexamethasone treatment. Indeed, addition of IGF-1 to either model of atrophy suppressed atrogin-1 mRNA to the level in untreated myotubes (FIG. 2a) and increased the levels of phosphorylated Foxo1, 3, and 4. On the other hand, in the starved cells, S6K remained largely dephosphorylated after IGF-1 addition, while in the dexamethasone-treated cultures, IGF-1 restored S6K phosphorylation. These findings suggest that S6K, while probably important in regulating other processes in muscle, is unlikely to regulate atrogin-1 expression.

To test directly whether IGF-1 suppresses atrogin-1 expression through the PI3K/AKT pathway, we used adenoviral vectors to introduce into myotubes a constitutively active and dominant-negative form of AKT (Brunet et al., 1999; Skurk et al., 2003). The constitutively active AKT mutant (c.a.AKT) contains the c-Src myristylation sequence fused in-frame to the N-terminus of an HA-tagged AKT coding sequence which activates AKT by targeting it to the membrane (Datta et al., 1999), while the dominant-negative AKT (d.n.AKT) contains the T308A and S473A mutations which prevent phosphorylation (Datta et al., 1999). Like IGF-1 treatment, expression of the constitutively active AKT prevented atrogin-1 induction by dexamnethasone (FIG. 2b) and blocked the reduction in phosphorylation of Foxo1, 3, and 4 (FIG. 2c). On the other hand, expression of the dominant-negative form of AKT slightly enhanced the dexamethasone-dependent induction of atrogin-1 (FIG. 2b) and did not affect the glucocorticoid-dependent dephosphorylation of the forkhead transcription factors (FIG. 2c). These findings further support the involvement of PI3K, AKT, and Foxo factors in mediating the effects of IGF-1 on atrogin-1 expression.

Foxo3 Dephosphorylation Induces Atrogin-1 Expression and the Atrogin-1 Promoter

To further explore the possible involvement of Foxo factors in atrogin-1 regulation, we utilized adenoviral constructs that produce wild type Foxo3 (FOXO3A), since Foxo3 is dephosphorylated following nutrient deprivation and dexamethasone treatment in myotubes (FIG. 1). We also used a constitutively active mutant of Foxo3 (c.a.FOXO3A) that is mutated in the three AKT phosphorylation sites, T32A, S253A, and S315A (Brunet et al., 1999). Each construct also expresses GFP, which enabled us to measure the efficiency of infection and to follow the morphological changes that occur in the myotubes as a result of expression of active Foxo3. In the myotubes, the wild type Foxo3 and the constitutively active mutant both induced a 6-fold increase in atrogin-1 mRNA (FIG. 3a), which is about twice as large as the induction upon dexamethasone treatment or with serum and nutrient deprivation. The addition of IGF-1 caused a 3-fold decrease in atrogin-1 mRNA content of cells expressing the wild type Foxo3, presumably as a result of IGF-1-induced AKT-mediated phosphorylation and inactivation of the wild type Foxo3. Accordingly, IGF-1 addition did not reduce the high level of atrogin-1 mRNA in cells expressing the constitutively active Foxo3, which cannot be phosphorylated.

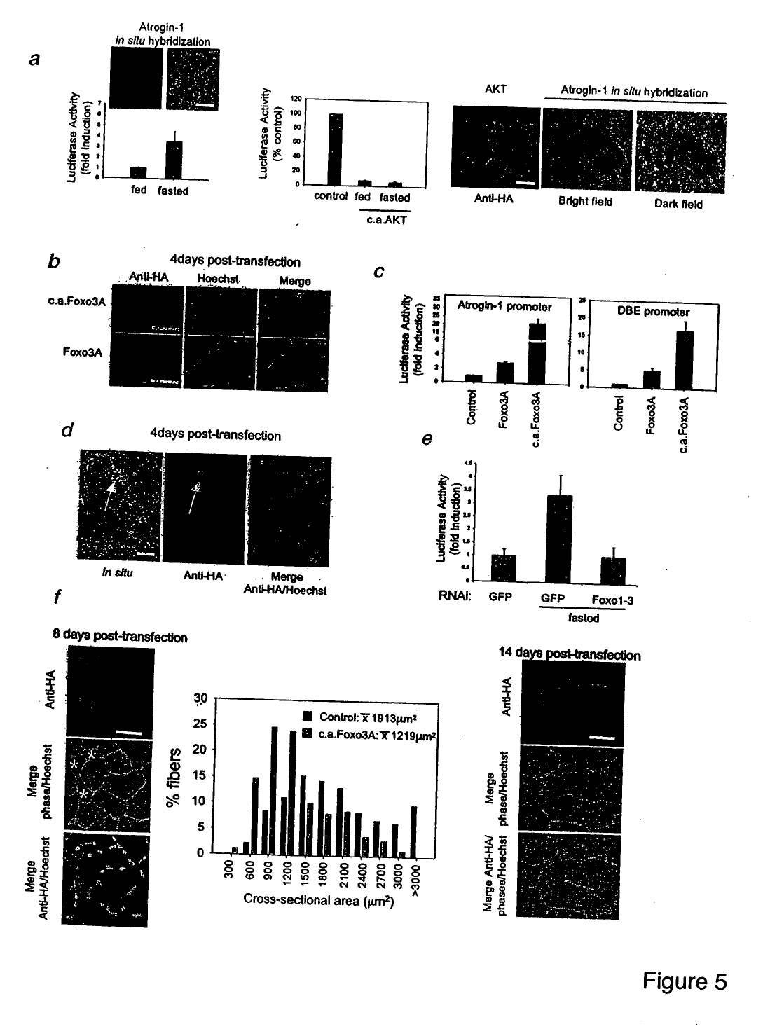

Further studies explored the effects of these treatments on the activity the atrogin-1 promoter. To prepare an atrogin-1 reporter gene construct, we cloned 3.5 kb of the mouse atrogin-1 5′ untranslated region behind the firefly luciferase gene and then made a further trucation of this region to create a 1.0 kb promoter fragment Wild type Foxo3 stimulated the activity of both 1.0 kb and 3.5 kb promoters when the myotubes were in the standard differentiation media (low serum). However, the 3.5 kb construct showed a 2.5-fold increase in activity, while the 1.0 kb construct showed only a 50% increase (FIG. 3b), presumably because the 3.5 kb construct contains 14 potential forkhead binding sites while the 1.0 kb reporter contains only 3 (see below, FIG. 5d). These findings also indicate that Foxo3 increases atrogin-1 mRNA content through enhanced transcription rather than some indirect effect on mRNA stability.

Influence of Foxo3 on Myotube Size and its Role in Atrophy

Atrogin-1 induction in vivo during atrophy occurs concomitantly with induction of MURF-1 and number of other atrophy-related genes (“atrogenes”), which may also be transcribed by Foxo factors (Jagoe et al., 2002; Lecker et al., 2004). Therefore, we tested whether activation of Foxo factors might trigger ‘atrophy’ of cultured myotubes. Morphological examination of cells expressing the wild type Foxo3 for 48 hrs demonstrated that Foxo3, while increasing atrogin-1 expression, also reduced the mean diameter of myotubes. Overexpression of the constitutively active Foxo3 caused an even more marked thinning of the myotubes. 48 hrs after infection, the mean diameter of these cells was 50% smaller than in myotubes infected with an adenovirus expressing only GFP (FIG. 3c).

These results further indicate that Foxo3 is likely to be a key transcription factor inducing the atrogin-1 gene and other key adaptations leading to atrophy in muscle cells. To test whether Foxo3 directly regulates atrogin-1 expression, we infected myotubes with a dominant-negative adenoviral construct for Foxo3 (d.n.FOXO3A). It had been shown previously that truncated Foxo1, as well as Foxo3 mutants lacking the transactivation domain, function as dominant-negative inhibitors of transcription by this family of factors (Hribal et al., 2003; Nakae et al., 2003; Nakae et al., 2001; Skurk et al., 2003). Overexpression of the dominant-negative mutant of Foxo3 led to a 30% decrease in the basal expression of atrogin-1, and decreased by half the induction of atrogin-1 mRNA by dexamethasone (FIG. 3d). Furthermore, the dominant-negative Foxo3, although it had no effect on myotube size alone, was able to prevent completely the decrease in cell size induced by dexamethasone. These findings indicate a key role of dephosphorylated Foxo3 in both catalyzing the transcription of atrogin-1 and in the initiation of muscle atrophy by glucocorticoids.

Other Downstream Targets of AKT do not Affect Atrogin-1 Expression

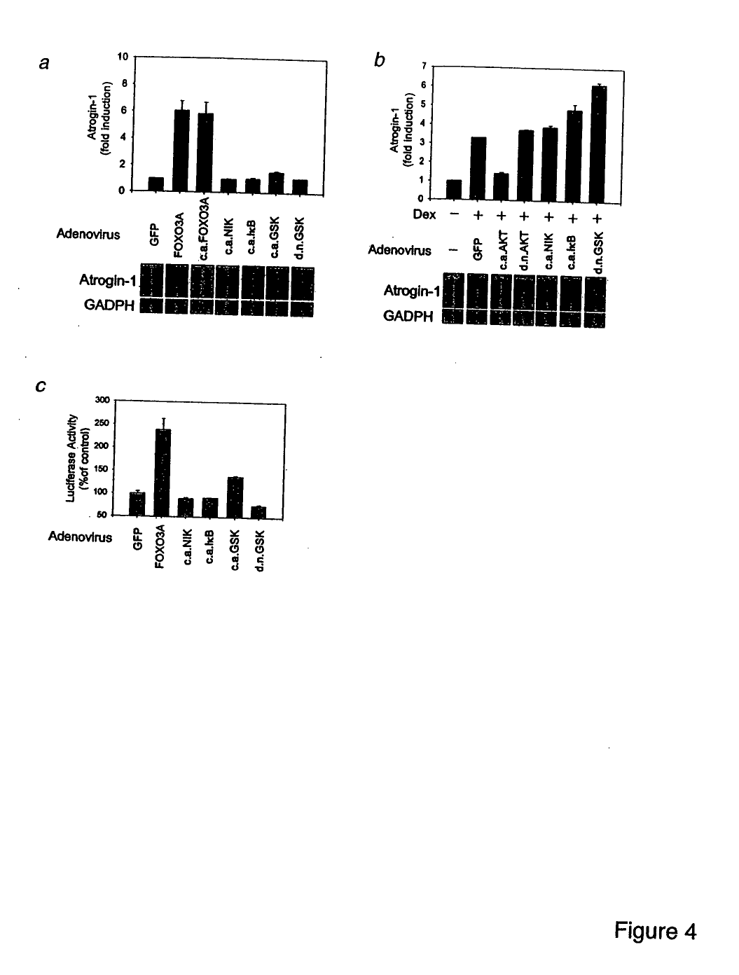

In order to learn if other AKT target proteins can also induce or suppress atrogin-1 expression, we examined several AKT targets that have been proposed to play a role in the regulation of muscle size. NFκB is the key transcription factor in the activation of inflammatory responses and has been suggested to mediate the effects of TNFα in inducing muscle wasting in sepsis and certain types of cachexia (Garcia-Martinez et al., 1994; Li and Reid, 2000). AKT has also been reported to activate NFκB by causing the phosphorylation of IKK, which acts in turn on IκB to trigger its degradation (Israel, 2000). IκB is the major inhibitor of NFκB entry into the nucleus and its degradation allows NFκB-mediated transcription. We therefore used adenoviral constructs to express components of the NFκB pathway in order to test if they may also affect atrogin-1 expression. However, no changes in levels of atrogin-1 mRNA were seen upon expression of either a constitutively active form of the NFκB-inducing kinase (c.a.NIK), winch functions as an activator of IKK, or expression of a constitutively active IκB (c.a.IκB) which is mutated at the site of phosphorylation S32A and S36A to prevent its phosphorylation and degradation (Winston et al., 1999). Thus, activation of NFκB or maintaining NFκB in an inactive form (FIG. 4a) did not influence basal atrogin-1 expression.

In addition, we measured the effect of GSK3β expression on atrogin-1 mRNA levels since GSK3β inhibition induces hypertrophy in myotubes (Rommel et al., 2001), and therefore, activation of GSK3β theoretically might be another possible mechanism contributing to atrophy. A constitutively active form of GSK3β (c.a.GSK), which has an S9A mutation at the site of AKT phosphorylation (Kim et al., 2002), induced a small (<2-fold) but reproducible increase in atrogin-1 expression that was much smaller than the 6-fold increase seen with Foxo dephosphorylation. However, a dominant-negative GSK3β, mutated in the kinase domain, (d.n.GSK) (Kim et al., 2002), did not affect atrogin-1 expression (FIG. 4a). Thus, in contrast to Foxo3, GSK3, (like NFκB) does not appear to have a major role in regulating atrogin-1 expression.

Additional experiments tested if altering the activity of NFκB or GSK3β might influence the induction of atrogin-1 by dexamethasone. Unlike constitutively active AKT, constitutively active NIK, the non-degradable IκB mutant, or the dominant-negative GSK3β did not block atrogin-1 induction (FIG. 4b). On the contrary, expression of this IκB mutant or the dominant-negative GSK3β consistently caused small (50%) increases in the induction by dexamethasone, although the physiological relevance of these effects are unclear. Since inhibition of GSK3β with dominant-negative mutants did not block or inhibit the response to dexamnethasone, GSK3β does not appear to mediate dexamethasone's effects on atrogin-1 expression. Furthermore, in related experiments, LiCl, an inhibitor of GSK3β, was also unable to decrease atrogin-1 induction by dexamethasone (data not shown). Finally, we tested the activity of the 3.5 kb atrogin-1 reporter in the presence of these NFκB and GSK3β constructs. These experiments confirmed the results of the Northern blot analysis shown above. Expression of Foxo3 and to a much lesser extent, GSK3β (but not constitutively active NIK or IκB), activated the atrogin-1 promoter (FIG. 4c). These findings together argue against a major role of GSK3β or NFκB in regulating atrogin-1 expression and indicate that AKT-dependent phosphorylation of Foxo3 accounts for the inhibition of atrogin-1 transcription by this pathway.

AKT Suppresses Atrogin-1 Expression in Adult Muscle.

In order to determine whether the IGF-1/AKT/Foxo pathway also regulates atrogin-1 expression and influences fiber size in fully differentiated muscle, as suggested by studies in cultured cells, we transfected adult mouse skeletal muscles by electroporation with the 3.5 kb atrogin-1 promoter-luciferase fusion and with constructs expressing members of the AKT/Foxo pathway. This technique was used by us to introduce multiple DNA constructs reproducibly into skeletal muscle fibers (Murgia et al., 2000; Pallafacchina et al., 2002; Serrano et al., 2001). Initial experiments demonstrated that the electroporated atrogin-1 reporter was regulated in a similar way to the endogenous atrogin-1 gene. 24 hours after of food deprivation, extracts from muscles transfected with the atrogin-1 reporter showed 3-4 fold more luciferase activity than extracts from muscles of fed animals, and the endogenous atrogin-1 gene was also induced as demonstrated by in situ hybridization (FIG. 5a), in accord with prior reports (Gomes et al., 2001). To determine whether AKT activation suppresses atrogin-1 expression in these muscles, we cotransfected the 3.5 kb atrogin-1 reporter with a constitutively active AKT mutant (Brunet et al., 1999). This active form of AKT markedly reduced the atrogin-1 promoter activity to 10% of its normal level. In addition, the constitutively active AKT completely inhibited the large increase in atrogin-1 promoter activity that is seen 24 h after food deprivation. Furthermore, in the muscles from food deprived mice, the AKT-transfected fibers contained little atrogin-1 message by in situ hybridization, while the surrounding untransfected fibers contained high levels of atrogin-1 mRNA (FIG. 5a). Thus, AKT activity is a key factor suppressing atrogin-1 expression in vivo and can even block its induction in low insulin, low IGF-1 states.

Foxo Induces Atrogin-1 Expression in Adult Myofibers.

Subsequent studies examined whether Foxo3 overexpression had similar effects on atrogin-1 expression in adult mouse muscles. After transfection, HA-tagged wild type Foxo3 was found by immunohistochemistry in both nuclei and cytoplasm, while the HA-tagged constitutively active Foxo3 mutant was present exclusively in nuclei (FIG. 5b). This accumulation in the nucleus can be explained by the reduced affinity of this mutant for the cytosolic 14-3-3 binding protein (13Brunet et al., 1999). Transfection of both the 3.5 kb atrogin-1 reporter and these Foxo3 constructs showed that the atrogin-1 promoter was activated 3-fold by wild type Foxo3 and more than 20-fold by the constitutively active Foxo3 (FIG. 5c). To ensure that the increased activity of the atrogin-1 reporter reflected Foxo3 function, a similar experiment was performed using as the reporter, the luciferase gene driven by 6 Foxo binding sites arrayed in tandem (DBE promoter, [1DAF-16 Binding Elements]) (FIG. 5c). This construct was activated to a similar extent by coexpression of wild type or constitutively active Foxo3 as was the atrogin-1 promoter. Furthermore, the fibers overexpressing Foxo3 also showed an increase in atrogin-1 mRNA levels as detected by in situ hybridization (FIG. 5d).

To test whether Foxo transcription factors also catalyze the induction of atrogin-1 in atrophying muscle during fasting, we used interference RNA (RNAi) to block the function of both Foxo1 and Foxo3 in fasted mice. Since expression of both Foxo1 and 3 increases in muscle in several catabolic states (Furuyama et al., 2003; Jagoe et al., 2002; Kamei et al., 2003; Lecker et al., 2004), and since both isoforms can activate the atrogin-1 promoter (FIG. 5c and data not shown), we used a Foxo region conserved in both isoforms as the RNAi vector. Electroporation of the RNAi for Foxo⅓ completely prevented the induction of the atrogin-1 promoter in these muscles 24 hrs after food deprivation. By contrast, a control RNAi (against GFP) did not affect the activation of the atrogin-1 promoter. Thus, Foxo factors are critical in the induction of atrogin-1 during fasting (FIG. 5e) and Foxo inactivation can account for the inhibition of this response by AKT.

Foxo3 Causes Muscle Atrophy in Adult Skeletal Muscle

Atrogin-1 is coordinately induced together with a number of other atrogenes during various types of atrophy (Lecker et al., 2004), possibly by activation of a common transcription factor(s). In order to test if Foxo3 not only promotes atrogin-1 transcription but might itself also lead to fiber atrophy, we transfected constitutively active Foxo3 into adult mouse skeletal muscle, and measured fiber size in the tibialis anterior muscle 8 days after transfection. Muscle fibers expressing c.aFOXO3A were identified by the presence of the HA epitope tag on the Foxo3 protein. As shown in FIG. 5f, the fibers expressing c.a.FOXO3A were much smaller than the untransfected surrounding fibers. Cross-sectional area was determined in more than 1,800 fibers taken from 8 muscles. Mean fiber size was markedly reduced (p<0.0 by T-test, assuming a normal distribution) (FIG. 5f). The fibers overexpressing c.a.FOXO3A had a cross-sectional area of 1219±56 μm² (median 1135 μm²) while that of nontransfected fibers was 1913±121 μm² (median 1754 μm²). This 35% decrease in area resembles the extent of atrophy seen in tibialis anterior after 1 week of denervation (Bodine et al., 2001b). Furthermore, in the muscles overexpressing c.a.FOXO3A, there was a particular enrichment in small fibers. These changes in fiber size in the presence of c.a.FOXO3A were even more dramatic after 14 days. At this time, c.a.FOXO3A-positive fibers contained minimal cytoplasm while the peripheral nuclei appeared normal. Since overexpression of atrogin-1 alone does not cause myotube or muscle atrophy (S.L. and M.S. unpublished results, D. Glass, personal communication), these findings suggest that Foxo3 induces not only atrogin-1 expression, but also other transcriptional changes leading to the enhanced protein breakdown that is necessary to account for such marked fiber shrinkage.

Forkhead Binding Sites are Located at 5′ End of the Atrogin-1 Gene.

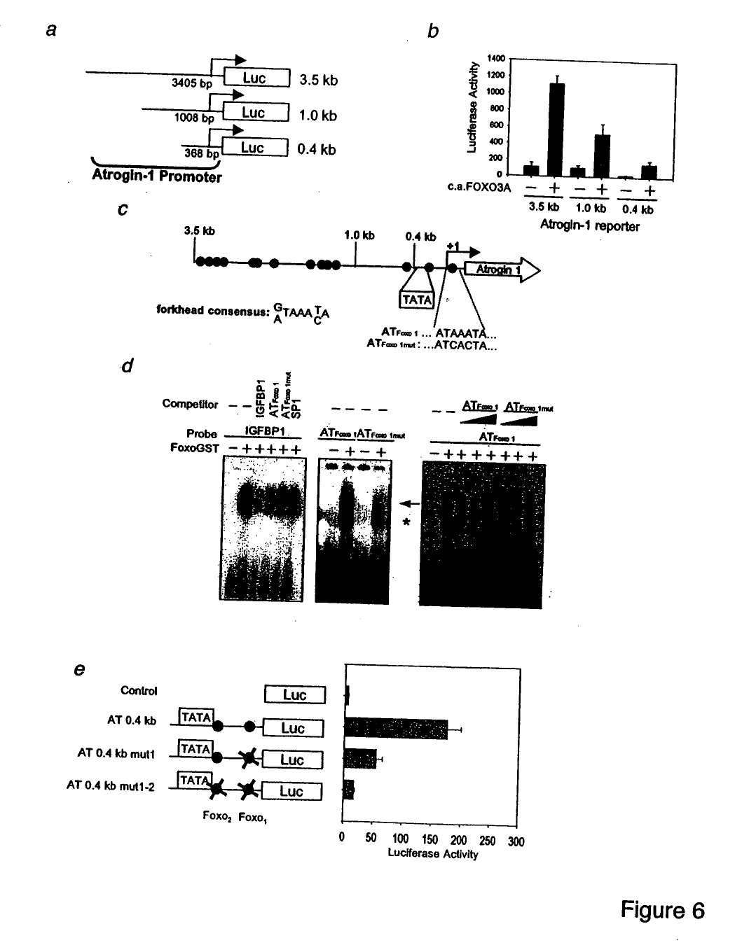

Since transcription from the atrogin-1 promoter appears to be catalyzed by forkhead transcription factors, we made a series of truncations of the promoter to isolate the important forkhead binding sites (FIG. 6a), and then transfected adult mouse skeletal muscles by electroporation with the atrogin-1 reporters with or without constructs expressing constitutively active Foxo3. This approach showed stimulation (˜10-fold) of the atrogin-1 promoter by constitutively active Foxo3 compared to similar experiments performed in myotube cultures (compare with FIG. 3b). Consequently, in vivo transfection studies simplified efforts to identify the key regulatory regions in the atrogin-1 promoter. When the promoter activity of each reporter was stimulated by coexpression of Foxo3, the extent of the induction appeared to correlate roughly with the length of the promoter construct, (i.e. presumably with the presence of multiple forkhead-sensitive regions throughout the 5′ untranslated region (FIG. 6b) (Brunet et al., 1999)). Surprisingly, even the smallest 5′ atrogin-1 fragment, which reduced the basal level of activity, responded to Foxo3, suggesting that Foxo3 binds to elements in that short region. A detailed analysis of that sequence revealed two potential forkhead binding sites. One was located just past the transcription start site (Foxo₁, +2), and the second partially overlapped the putative TATA box at −94 relative to the transcription start site (Foxo₂) (FIG. 6c). The ability of forkhead factors to bind to these sites was therefore tested in an electrophoretic mobility shift assay using a purified forkhead fusion protein (FoxoGST) (FIG. 6d). FoxoGST bound to a control oligonucleotide containing a known forkhead site from the IGFBP1 promoter. A 100-fold molar excess of unlabeled IGFBP1 oligonucleotide prevented formation of the FoxoGST-IGFBP1 complex. Formation of the FoxoGST-IGFBP1 complex was also markedly reduced by an unlabeled oligonucleotide containing one of the putative forkhead binding sites in the atrogin-1 promoter, ATAAATA (AT_{Foxo 1}). By contrast, a mutated version of this site, ATCACTA (AT_{Foxo 1mut}), as well as an unrelated oligonucleotide containing an SP1 binding site, did not prevent complex formation. FoxoGST also bound to the oligonucleotide containing the Foxo₁ site but did not bind to the mutated version. Finally, in a competition assay, increasing amounts of the unlabeled AT_{Foxo 1} oligonucleotide, but not of the mutated version, were able to block FoxoGST-AT _{Foxo 1} complex formation.

These results confirm that forkhead factors are capable of binding directly to the atrogin-1 5′untranslated region in close proximity to both the putative TATA box and the initiation site of transcription. While it is unusual for elements involved in transcriptional regulation to be found in the 5′ untranslated regions of genes, functionally important MEF2 sites have been found distal to the TATA box in the troponin I gene (Di Lisi et al., 1998), and functional Foxo sites have been identified in the first intron of bim gene (Gilley et al., 2003).

Finally, we investigated whether the two forkhead binding sites present in the short 0.4 kb fragment of the atrogin-1 promoter are necessary for activation by Foxo3. We used site directed mutagenesis to introduce into the 0.4 kb reporter mutations in Foxo₁ and both Foxo₁ and Foxo₂ (FIG. 6e). Adult skeletal muscles were then transfected with the mutated reporters and the constitutively active Foxo3 vector, and luciferase activity determined in extracts from the muscles 4 days later. The single mutation in Foxo₁ reduced Foxo3-mediated activation by 70% below the level seen with the wild type 0.4 kb atrogin-1 promoter, and almost no activation was observed with the double mutant that lacked both forkhead binding sites. These experiments show by a combination of different approaches that Foxo3 binds to the atrogin-1 promoter and activates its transcription.

### DISCUSSION

Foxo and Atrophy

The discovery that dephosphorylation and activation of Foxo transcription factors leads to atrogin-1 expression and profound atrophy of muscle cells represents a major advance in our understanding of the molecular mechanisms of muscle atrophy. Specifically, we have shown that Foxo3 activation by itself can cause a large stimulation in atrogin-1 transcription, and can lead to dramatic decrease in the cross sectional area of mouse muscle fibers. Moreover, the induction of atrogin-1 and myotube atrophy by glucocorticoids, and atrogin-1 induction in mice upon fasting could be blocked by dominant negative inhibitors or RNAi Foxo3 constructs. Thus, activation of Foxo3 is both necessary and sufficient for these responses. It remains to be established whether Foxo1, which is regulated in a similar fashion as Foxo3 upon starvation and dexamethasone treatment, and Foxo4, which is also expressed in muscle, are also essential for these responses.

These experiments are the first to implicate a specific transcription factor in the expression of genes necessary for rapid atrophy, and thus demonstrate a new function for the Foxo (forkhead) family. It is well established that these transcription factors play an important role in the control of the cell cycle and in the initiation of apoptosis (Ramaswamy et al., 2002; Tran et al., 2003). Because of these additional functions, simple transduction of Foxo3 into myoblast cultures could have led to results unrelated to atrophy (e.g. apoptosis). Our approach of viral infection of cultured myotubes and direct gene transfer by electroporation into adult muscle allowed us to analyze the effects of Foxo3 in differentiated, post-mitotic tissue and to circumvent the potential problems that Foxo3 expression can have during muscle development.

Dephosphorylation of forkhead family members is the key event allowing their translocation into the nucleus and the transcription of target genes (Brunet et al., 1999; Tran et al., 2003). In a variety of organisms and tissues, AKT and downstream targets can affect cell size. For instance, loss of drosophila AKT leads to smaller organs due to a reduction in cell size and number (Montagne et al., 1999; Verdu et al., 1999). In addition, overexpression of the Foxo homolog in drosophila, DAF16, leads to small flies with reduced cell numbers and a phenotype resembling starvation (Kramer et al., 2003; Puig et al., 2003). Inhibition of Foxo family members by phosphorylation is also required for muscle cell differentiation and fusion of myoblasts into myotubes (Hribal et al., 2003); however, the specific roles of individual Foxo1, 3 and 4 isoforms in mammalian muscle is less clear. All three Foxo family members are dephosphorylated in muscle culture in response to nutrient and serum deprivation or dexamethasone, and this response (like overexpression of Foxo3) induces atrogin-1 expression and favors myotube atrophy. Accordingly, Foxo3 inhibition by a dominant-negative mutant decreases atrogin-1 mRNA and prevents its induction by dexamethasone (FIG. 3d). Furthermore, constitutively active AKT not only inactivates these three Foxo family members in mice, but also prevents the dramatic induction of atrogin-1 in vitro and in vivo. In adult muscle, we showed that Foxo3 binds directly to the atrogin-1 promoter, which contains 14 potential forkhead binding sites, and thus activates transcription of this gene.

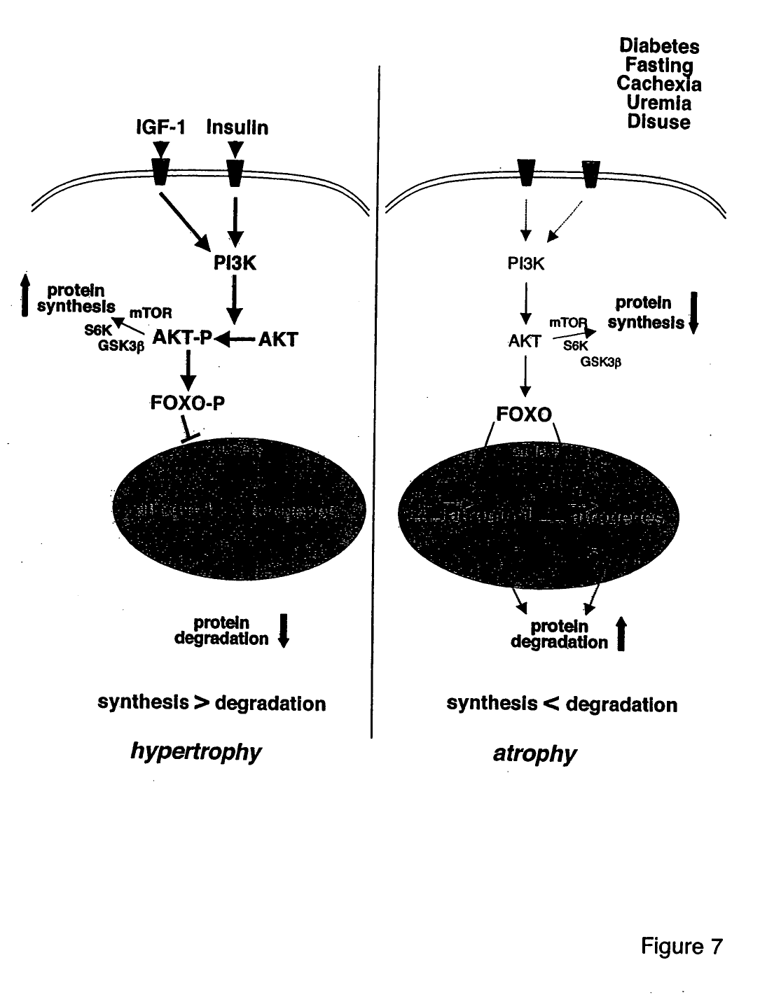

The present findings and related ones from this laboratory (Sacheck et al., manuscript submitted) have uncovered an important new action of IGF-1 and insulin that is likely to contribute to their capacity to promote muscle growth—their ability to suppress atrogin-1 expression and the activation of the transcriptional program for muscle atrophy. While the ability of these hormones to stimulate protein synthesis in muscle through activation of PI3K and AKT is widely appreciated (Grizard et al., 1999; Rommel et al., 2001; Svanberg et al., 1996), IGF-1 and insulin also reduce overall protein breakdown, especially the degradation of myofibrillar proteins, and block the expression of the key atrophy-related ubiquitin ligases, MuRF-1 and atrogin-1 (Sacheck et al., manuscript submitted). Upon addition of IGF-1 or insulin, the fall in atrogin-1 mRNA is dramatic and rapid, due largely to its short half-life (Sacheck et al., manuscript submitted). Since atrogin-1 expression is essential for atrophy (Bodine et al., 2001a) and correlates with the extent of proteolysis in myotubes (Sacheck et al., manuscript submitted), this decrease in atrogin-1 expression per se should reduce the rate and extent of atrophy. By causing phosphorylation of AKT and the forkhead family of transcription factors (Foxo1,3, and 4), IGF-1 and other growth factors presumably block not only atrogin-1 but also expression of other key atrogenes whose expression contributes to muscle atrophy (FIG. 7).

Thus IGF-1, in promoting growth, both enhances overall protein synthesis and suppresses atrogin-1 expression and proteolysis. By contrast, in catabolic conditions where IGF-1 or insulin are low (e.g. fasting or diabetes) and presumably also in conditions where there is resistance to their actions (e.g. cancer, uremia, sepsis, diabetes, Cushing's syndrome) AKT is dephosphorylated and its activity is reduced below control levels. Denervation and disuse also have been reported to result in reduced AKT activity (Bodine et al., 2001b). Consequently, all these states should involve dephosphorylation of Foxo transcription factors leading to their translocation into the nucleus where they activate transcription of atrogin-1 and presumably other atrogenes. At the same time, decreased AKT activity reduces protein synthesis because of the dephosphorylation of GSK, mTOR and S6K. Together, these adaptations lead to a rapid decrease in myofiber size (FIG. 7). In addition, to trigger profound muscle atrophy, Foxo must also be activating the breakdown of myofibrillar proteins, a characteristic feature of atrophying muscles. On the other hand, when IGF-1 or insulin levels are high, and when AKT is active, rates of protein synthesis rise through activation of mTOR and S6K, and Foxo transcription factors remain phosphorylated in the cytosol and thus cannot activate the transcription of key atrogenes. These conditions favor muscle protein synthesis, low rates of proteolysis, net protein accumulation, and fiber hypertophy.

In muscles atrophying due to fasting, diabetes, uremia and cancer, Foxo 1 and 3 mRNA levels rise (Furuyama et al., 2003; Kamei et al., 2003; Lecker et al., 2004). These findings strongly suggest that atrophy in these cachectic states occurs through activation of forkbead factors. Although Foxo3 activation can cause a reduction in myofiber size, it remains to be studied what specific effects Foxo1 and Foxo4 have when overexpressed, and whether they might control other genes necessary for the development of atrophy. Further experiments are thus needed to determine which of the recently identified atrogenes are controlled directly or indirectly by these factors. Recent studies by Glass and colleagues have indicated that Foxo family members also catalyze the expression of MuRF-1, the other E3 that is induced by glucocorticoids and suppressed by IGF-1 (D. Glass, personal communication). Although MuRF-1 mRNA rises and falls more slowly and less dramatically than atrogin-1 mRNA and does not correlate with proteolysis (Sacheck et al., manuscript submitted), it too plays a key role in atrophy.

Together these findings indicate that Foxo1- and 3-dependent transcription is activated by two mechanisms in atrophying muscles: 1) their mRNA levels rise in all types of atrophy examined, presumably through increased transcription (Jagoe et al., 2002; Lecker et al., 2004), and 2) as shown here, the transcription factors are dephosphorylated due to the suppression of AKT activity (and perhaps by other mechanisms). Amongst the most induced genes in our recent analysis of the transcriptional changes in muscle atrophying due to fasting as well as in diabetes, uremia and cancer cachexia (Jagoe et al., 2002; Lecker et al., 2004) are PDK4, p21, Gadd45, 4E-BP1, all of which have recently been shown to be transcribed by Foxo factors in mammalian or insect cells (Furuyama et al., 2003; Nakae et al., 2003; Puig et al., 2003; Tran et al., 2002). Moreover, Foxo family have been recently implicated in the development of insulin resistance (Nakae et al., 2002), a prominent feature of muscles in uremia, cancer cachexia, fasting, as well as diabetes (Zierath et al., 2000). This resistance should alleviate the inhibition of expression of atrogin-1, MuRF-1 and other atrogenes and also the inhibition of protein degradation by circulating insulin and IGF-1. Another transcriptional change which we observed in these catabolic states that may also contribute to IGF-1 resistance and Foxo activation is a reduction in mRNA for IGF binding protein 5, an enhancer of IGF-1 function (Lecker et al., 2004; Schneider et al., 2002).

Glucocorticoids are also required for muscle atrophy and the enhanced proteolysis in many systemic diseases (Hasselgren, 1999). In accord with the present findings, the sensitivity to the catabolic actions of glucocorticoids is decreased by insulin in incubated rat muscles (Wing and Goldberg, 1993) as well as in myotubes (Sacheck et al., manuscript submitted). However, no glucocorticoid-response elements are present in the atrogin-1 promoter. Therefore, glucocorticoids must act indirectly, perhaps by inducing the expression of key proteins that in turn activate transcription. The obvious candidates for such regulators are Foxo family members. In fact, six hours after administration of glucocorticoids, Foxo1 and 3 mRNA rise in skeletal muscle (Furuyama et al., 2003), as occurs in various types of atrophying muscles (Lecker et al., 2004). Foxo members and glucocorticoids function together in transcription of genes in liver cells (Nakae et al., 2001; Nasrin et al., 2000). It is noteworthy that in fasting, when glucocorticoids favor the net release of amino acids from muscle, they enhance the liver's capacity to convert amino acids into glucose, and some of the gluconeogenic actions of glucocorticoids in the liver require Foxo1 and 3. For example, glucocorticoids induce expression of glucose-6-phosphatase and PEPCK in the liver, and Foxo1 mediates the suppression by insulin on glucose-6-phosphatase (Nakae et al., 2001). Foxo1 and 3 also work as cofactors with glucocorticoids, perhaps by recruiting the p300/CBP/SRC coactivator complex to the forkhead binding site Nasrin et al., 2000). Glucocorticoids, then, may promote atrogin-1 expression and muscle atrophy by simply inducing Foxo production or may indirectly promote atrogin-1 expression by recruiting other factors like p300/CBP to act with Foxo1 or 3 on the atrogin-1 promoter.

Other Possible Regulators of Atrogin-1 Expression

Other kinases downstream of AKT, including GSK3β, as well as the MEK and calcineurin systems, have all been implicated in the regulation of muscle fiber size (Bodine et al., 2001a; Murgia et al., 2000; Musaro et al., 1999). However, in contrast to AKT, none of these factors appear to have direct roles in the regulation of atrogin-1 expression, The present findings with Foxo3 transcription can account for the prior observation that inhibition of AKT by a dominant-negative mutant induces myotube atrophy (Bodine et al., 2001b; Pallafacchina et al., 2002). By contrast, the lack of inhibition of the atrogin-1 induction by dexamethasone by either the dominant-negative GSK3β (FIG. 3b) or LiCl treatment (data not shown) seems to rule out a major role for GSK3β in regulation of atrogin-1 transcription. Atrogin-1 induction appears to occur independently of mTOR/S6K since even when IGF-1 was added, starvation of cells for nutrients and serum caused an almost complete dephosphorylation of mTOR/S6K yet atrogin-1 was induced in the starved cells only in the absence of IGF-1 (FIG. 2a). Most likely, in these starved cells, as in fasting in vivo (Li and Goldberg, 1976), rates of translation decrease by different mechanisms (i.e. mTOR/S6K and GSK3β dephosphorylation) from the concomitant Foxo-dependent activation of atrogin-1 transcription. Accordingly, inhibtion of PI3K by either pharmacological (e.g. LY294002) or genetic means (e.g. dominant-negative AKT or constitutively active SHIP) leads to atrophic myotubes (Rommel et al., 2001), while pharmacological inhibition of mTOR by rapamycin does not induce atrophy in culture or in vivo (Bodine et al., 2001b; Pallafacchina et al., 2002; Rommel et al., 2001).

While the PI3K/AKT pathway is clearly critical in determining whether a muscle grows or atrophies (FIG. 7), it does not influence muscle size simply through regulation of protein synthesis, as had been generally believed (Glass, 2003). It is noteworthy that constitutively active AKT, like growth hormone (i.e. IGF-1) administration (Goldberg, 1969), can induce net growth of even denervated muscle (Bodine et al., 2001b; Pallafacchina et al., 2002), but this response is completely blocked by treatment with rapamycin, suggesting that it is dependent only on the enhancement of protein synthesis. On the other hand, mTOR/S6K and GSK3β still appear to have some indirect effect on atrogin-1 expression. In myotubes, rapamycin causes a small induction of atrogin-1 (Sacheck et al., manuscript submitted) and constitutively active GSK3β (FIG. 3b) consistently caused a small induction of atrogin-1 expression. Thus, there appears to be additional modes of regulation of this important gene.

The proinflammatory cytokine TNFα has also been proposed to stimulate muscle atrophy in sepsis and certain types of cancer (Garcia-Martinez et al., 1994; Li and Reid, 2000). In many cells, TNFα triggers the expression of inflammatory mediators through activation of the transcription factor NFκB (Li and Reid, 2000). Based on observations in myoblast differentiation, NFκB had been proposed to function as a key transcription factor that may cause muscle atrophy and cachexia (Guttridge et al., 2000). Our findings upon transfection of an activator of NFκB or a dominant-negative IκB mutant exclude an important role for NFκB in control of atrogin-1 transcription. On the contrary, expression of a dominant-negative IκB, which inhibits NFκB activity, actually caused a small increase in atrogin-1 mRNA, and our gel-shift experiments showed a slight decrease in NFκB content in nuclear extracts from dexamethasone-treated myotubes (data not shown) perhaps because dexamethasone can induce IκB expression (Du et al., 2000). In addition, in myotubes treated with hydrogen peroxide, expression of atrogin-1 rose but this response did not correlate with NFκB activation (Li et al., 2003a). Thus, NFκB1 does not appear to be directly involved in atrogin-1 expression, although in some types of muscle wasting, TNFα may promote atrophy perhaps by activating other components of the Ub-proteasome system (Li et al., 2003b), or by effects inducing insulin resistance (Peraldi and Spiegelman, 1998). It is also noteworthy that MEK and calcineurin inhibitors do not affect atrogin-1 expression (Sacheck et al., manuscript submitted), which is consistent with the recent reports that these pathways influence fiber type composition but not fiber size (Murgia et al., 2000; Pallafacchina et al., 2002; Serrano et al., 2001) and that neither calcineurin inhibitors nor dominant-negative mutants for Ras and MEK inhibitors induce atrophy (Pallafacchina et al., 2002; Rommel et al., 2001; Serrano et al., 2001).

While defining new roles for Foxo3 and the IGF-1/PI3K/AKT pathways in the control of muscle size, the present findings have also generated many important new questions for study. For instance, what additional genes are induced (or suppressed) in atrophying muscles by activation of forkhead factors? Are Foxo1 and 4 also required for atrophy and if so, what are their respective roles in different types of muscle wasting? How are the levels of phosphorylated Foxo and the phosphorylation states of intermediates in the IGF-1/PI3K/AKT pathway influenced by contractile activity, by disuse and denervation? Increased contractile work does stimulate IGF production (McKoy et al., 1999), and its autocrine actions may inhibit Foxo activation. Finally, the precise roles of atrogin-1 (and MURF-1) in the development of muscle wasting (e.g. the nature of its substrates) are still obscure. Despite these uncertainties, the identification of Foxo3 as a major activator of atrogin-1 and muscle wasting following glucocorticoid administration or nutrient deprivation suggest new potential points of pharmacological intervention to prevent or diminish this debilitating process.
Attaix, D., Combaret, L., Pouch, M. N., and Taillandier, D. (2001). Regulation of proteolysis. Curr Opin Clin Nutr Metab Care 4, 45-49.
Bodine, S. C., Latres, E., Baumhueter, S., Lai, V. K., Nunez, L., Clarke, B. A., Poueyrirou, W. T., Panaro, F. J., Na, E., Dharmarajan, K., et al. (2001a). Identification of ubiquitin ligases required for skeletal muscle atrophy. Science 294, 1704-1708.
Bodine, S. C., Stitt, T. N., Gonzalez, M.; Kline, W. O., Stover, G. L., Bauerlein, R., Zlotchenko, E., Scrimgeour, A., Lawrence, J. C., Glass, D. J., and Yancopoulos, G. D. (2001b). Akt/mTOR pathway is a crucial regulator of skeletal muscle hypertrophy and can prevent muscle atrophy in vivo. Nat Cell Biol 3, 1014-1019.
Booth, F. W., and Criswell, D. S. (1997). Molecular events underlying skeletal muscle atrophy and the development of effective countermeasures. Int J Sports Med 18 Suppl 4, S265-269.
Brummelkamp, T. R., Bernards, R., and Agami, R. (2002). A system for stable expression of short interfering RNAs in mammalian cells. Science 296, 550-553.
Brunet, A., Bonni, A., Zigmond, M. J., Lin, M. Z., Juo, P., Hu, L. S., Anderson, M. J., Arden, K. C., Blenis, J., and Greenberg, M. E. (1999). Akt promotes cell survival by phosphorylating and inhibiting a Forkhead transcription factor. Cell 96, 857-868.
Church, G. M., and Gilbert, W. (1984). Genomic sequencing. Proceedings of the National Academy of Sciences of the United States of America 81, 1991-1995.
Datta, S. R., Brunet, A., and Greenberg, M. E. (1999). Cellular survival: a play in three Akts. Genes Dev 13, 2905-2927.
DeVol, D. L., Rotwein, P., Sadow, J. L., Novakofski, J., and Bechtel, P. J. (1990). Activation of insulin-like growth factor gene expression during work-induced skeletal muscle growth. Am J Physiol 259, E89-95.
Di Lisi, R., Millino, C., Calabria, E., Altruda, F., Schiaffino, S., and Ausoni, S. (1998). Combinatorial cis-acting elements control tissue-specific activation of the cardiac troponin I gene in vitro and in vivo. J Biol Chem 273, 25371-25380.
Furuyama, T., Nakazawa, T., Nakano, I., and Mori, N. (2000). Identification of the differential distribution patterns of mRNAs and consensus binding sequences for mouse DAF-16 homologues. Biochem J 349, 629-634.
Garcia-Martinez, C., Llovera, M., Agell, N., Lopez-Soriano, F. J., and Argiles, J. M. (1994). Ubiquitin gene expression in skeletal muscle is increased by tumour necrosis factor-alpha. Biochemical & Biophysical Research Communications 201, 682-686.
Gilley, J., Coffer, P. J., and Ham, J. (2003). FOXO transcription factors directly activate bim gene expression and promote apoptosis in sympathetic neurons. J Cell Biol 162, 613-622.
Glass, D. J. (2003). Signalling pathways that mediate skeletal muscle hypertrophy and atrophy. Nat Cell Biol 5, 87-90.
Goldberg, A. L. (1969). Protein turnover in skeletal muscle. I. Protein catabolism during work-induced hypertrophy and growth induced with growth hormone. Journal of Biological Chemistry 244, 3217-3222.
Gomes, M. D., Lecker, S. H., Jagoe, R. T., Navon, A., and Goldberg, A. L. (2001). Atrogin-1, a muscle-specific F-box protein highly expressed during muscle atrophy. Proc Natl Acad Sci USA 98, 14440-14445.
Grizard, J., Dardevet, D., Balage, M., Larbaud, D., Sinaud, S., Savary, I., Grzelkowska, K., Rochon, C., Tauveron, I., and Obled, C. (1999). Insulin action on skeletal muscle protein metabolism during catabolic states. Reprod Nutr Dev 39, 61-74.
Guttridge, D. C., Mayo, M. W., Madrid, L. V., Wang, C. Y., and Baldwin, A. S., Jr. (2000). NF-kappaB-induced loss of MyoD-messenger RNA: possible role in muscle decay and cachexia. Science 289, 2363-2366.
Hara, K., Maruki, Y., Long, X., Yoshino, K., Oshiro, N., Hidayat, S., Tokunaga, C., Avruch, J., and Yonezawa, K. (2002). Raptor, a binding partner of target of rapamycin (TOR), mediates TOR action. Cell 110, 177-189.
Hasselgren, P. O. (1999). Glucocorticoids and muscle catabolism. Current Opinion in Clinical Nutrition & Metabolic Care 2, 201-205.
Israel, A. (2000). The IKK complex: an integrator of all signals that activate NF-kappaB? Trends Cell Biol 10, 129-133.
Jagoe, R. T., and Goldberg, A. L. (2001). What do we really know about the ubiquitin-proteasome pathway in muscle atrophy? Current opinion in Clinical Nutrition and Metabolic Care 4, 183-190.
Jagoe, R. T., Lecker, S. H., Gomes, M., and Goldberg, A. L. (2002). Patterns of gene expression in atrophying skeletal muscles: the response to food deprivation. FASEB Journal 16,1697-1712.
Kamei, Y., Mizukami, J., Miura, S., Suzuki, M., Takahashi, N., Kawada, T., Taniguchi, T., and Ezaki, 0. (2003). A forkhead transcription factor FKHR up-regulates lipoprotein lipase expression in skeletal muscle. FEBS Lett 536, 232-236.
Kayali, A. G., Young, V. R., and Goodman, M. N. (1987). Sensitivity of myofibrillar proteins to glucocorticoid-induced muscle proteolysis. Am J Physiol 252, E621-626.
Kettelhut, I. C., Wing, S. S., and Goldberg, A. L. (1988). Endocrine regulation of protein breakdown in skeletal muscle. Diabetes-Metabolism Reviews 4, 751-772.
Kim, D. H., Sarbassov dos, D., Ali; S. M., Latek, R. R., Guntur, K. V., Erdjument-Bromage, H., Tempst, P., and Sabatini, D. M. (2003). GbetaL, a positive regulator of the rapamycin-sensitive pathway required for the nutrient-sensitive interaction between raptor and mTOR. Mol Cell 11, 895-904.
Kim, H. S., Skurk, C., Thomas, S. R., Bialik, A., Suhara, T., Kureishi, Y., Birnbaum, M., Keaney, J. F., Jr., and Walsh, K. (2002). Regulation of angiogenesis by glycogen synthase kinase-3beta. J Biol Chem 277, 41888-41896.
Kramer, J. M., Davidge, J. T., Lockyer, J. M., and Staveley, B. E. (2003). Expression of Drosophila FOXO regulates growth and can phenocopy starvation. BMC Dev Biol 3, 5.
Lecker, S. H., Jagoe, R. T., Gilbert, A., Gomes, M. G., V., B., Bailey, J. L., Price, S. R., Mitch, W. E., and Goldberg, A. L. (2004). Multiple types of skeletal muscle atrophy involve a common program of changes in gene expression. Faseb J 18, in press.
Lecker, S. H., Solomon, V., Mitch, W. E., and Goldberg, A. L. (1999). Muscle protein breakdown and the critical role of the ubiquitin-proteasome pathway in normal and disease states. Journal of Nutrition 129, 227-S-237S.
Li, J.B., and Goldberg, A.L. (1976). Effects of food deprivation on protein synthesis and degradation in rat skeletal muscles. American Journal of Physiology 231, 441-448.
Li, Y. P., Chen, Y., Li, A. S., and Reid, M. B. (2003a). Hydrogen peroxide stimulates ubiquitin-conjugating activity and expression of genes for specific E2 and E3 proteins in skeletal muscle myotubes. Am J Physiol Cell Physiol 285, C806-812.
Li, Y. P., Lecker, S. H., Chen, Y., Waddell, I. D., Goldberg, A. L., and Reid, M. B. (2003b). TNF-alpha increases ubiquitin-conjugating activity in skeletal muscle by up-regulating UbcH2/E220k. Faseb J 17,1048-1057.
Li, Y. P., and Reid, M. B. (2000). NF-kappaB mediates the protein loss induced by TNF-alpha in differentiated skeletal muscle myotubes. Am J Physiol Regul Integr Comp Physiol 279, RI 165-1170.
McKoy, G., Ashley, W., Mander, J., Yang, S. Y., Williams, N., Russell, B., and Goldspink, G. (1999). Expression of insulin growth factor-1 splice variants and structural genes in rabbit skeletal muscle induced by stretch and stimulation. J Physiol 516 (Pt 2), 583-592.
Mitch, W. E., Bailey, J. L., Wang, X., Jurkovitz, C., Newby, D., and Price, S. R. (1999). Evaluation of signals activating ubiquitin-proteasome proteolysis in a model of muscle wasting. Am J Physiol 276, C1132-1138.
Montagne, J., Stewart, M. J., Stocker, H., Hafen, E., Kozma, S. C., and Thomas, G. (1999). Drosophila S6 kinase: a regulator of cell size. Science 285, 2126-2129.
Murgia, M., Serrano, A. L., Calabria, E., Pallafacchina, G., Lomo, T., and Schiaffino, S. (2000). Ras is involved in nerve-activity-dependent regulation of muscle genes. Nat Cell Biol 2, 142-147.
Musaro, A., McCullagh, K. J., Naya, F. J., Olson, E. N., and Rosenthal, N. (1999). IGF-1 induces skeletal myocyte hypertrophy through calcineurin in association with GATA-2 and NF-ATc1. Nature 400, 581-585.
Nakae, J., Biggs, W. H., Kitamura, T., Cavenee, W. K., Wright, C. V., Arden, K. C., and Accili, D. (2002). Regulation of insulin action and pancreatic beta-cell function by mutated alleles of the gene encoding forkhead transcription factor Foxo1. Nat Genet 32, 245-253.
Nakae, J., Kitamura, T., Kitamura, Y., Biggs, W. H., 3rd, Arden, K. C., and Accili, D. (2003). The forkhead transcription factor Foxo1 regulates adipocyte differentiation. Dev Cell 4, 119-129.
Nakae, J., Kitmura, T., Silver, D.L., and Accili, D. (2001). The forkhead transcription factor Foxo1 (Fkhr) confers insulin sensitivity onto glucose-6-phosphatase expression. J Clin Invest 108, 1359-1367.
Nasrin, N., Ogg, S., Cahill, C. M., Biggs, W., Nui, S., Dore, J., Calvo, D., Shi, Y., Ruvkun, G., and Alexander-Bridges, M. C. (2000). DAF-16 recruits the CREB-binding protein coactivator complex to the insulin-like growth factor binding protein 1 promoter in HepG2 cells. Proc Natl Acad Sci U S A 97, 10412-10417.
Pallafacchina, G., Calabria, E., Serrano, A. L., Kalhovde, J. M., and Schiaffino, S. (2002). A protein kinase B-dependent and rapamycin-sensitive pathway controls skeletal muscle growth but not fiber type specification. Proc Natl Acad Sci USA 99, 9213-9218.
Peng, T., Golub, T. R., and Sabatini, D. M. (2002). The immunosuppressant rapamycin mimics a starvation-like signal distinct from amino acid and glucose deprivation. Mol Cell Biol 22, 5575-5584.
Peraldi, P., and Spiegelman, B. (1998). TNF-alpha and insulin resistance: summary and future prospects. Mol Cell Biochem 182, 169-175.
Peterson, R. T., Desai, B. N., Hardwick, J. S., and Schreiber, S. L. (1999). Protein phosphatase 2A interacts with the 70-kDa S6 kinase and is activated by inhibition of FKBP12-rapamycin associated protein. Proc Nat! Acad Sci U S A 96, 4438-4442.
Puig, O., Marr, M. T., Ruhf, M. L., and Tjian, R. (2003). Control of cell number by Drosophila FOXO: downstream and feedback regulation of the insulin receptor pathway. Genes Dev 17, 2006-2020.
Puigserver, P., Rhee, J., Donovan, J., Walkey, C. J., Yoon, J. C., Oriente, F., Kitamura, Y., Altomonte, J., Dong, H., Accili, D., and Spiegelman, B. M. (2003). Insulin-regulated hepatic gluconeogenesis through FOXO1-PGC-1 alpha interaction. Nature 423, 550-555.
Ramaswamy, S., Nakamura, N., Sansal, I., Bergeron, L., and Sellers, W. R. (2002). A novel mechanism of gene regulation and tumor suppression by the transcription factor FKHR. Cancer Cell 2, 81-91.
Rommel, C., Bodine, S. C., Clarke, B. A., Rossman, R., Nunez, L., Stitt, T. N.,Yancopoulos, G. D., and Glass, D. J. (2001). Mediation of IGF-1-induced skeletal myotube hypertrophy by PI(3)K/Akt/mTOR and PI(3)K/Akt/GSK3 pathways. Nat Cell Biol 3, 1009-1013.
Schneider, M. R., Wolf, E., Hoeflich, A., and Lahm, H. (2002). IGF-binding protein-5: flexible slaver in the IGF system and effector on its own. J Endocrinol 172, 423-440.
Serrano. A. L., Murgia, M., Pallafacchina, G., Calabria, E., Coniglio, P., Lomo, T., and Schiaffino, S. (2001). Calcineurin controls nerve activity-dependent specification of slow skeletal muscle fibers but not muscle growth. Proc Nati Acad Sci U S A 98, 13108-13113.
Skurk, C., Maatz, H., Kim, H. S., Yang, J., Abid, M. R., Aird, W. C., and Walsh, K. (2003). The Akt-regulated forkhead transcription factor FOXO3a controls endothelial cell viability through modulation of the caspase-8 inhibitor FLIP. J Biol Chem.
Solomon, V., Baracos, V., Sarraf, P., and Goldberg, A. L. (1998). Rates of ubiquitin conjugation increase when muscles atrophy, largely through activation of the N-end rule pathway. Proceedings of the National Academy of Sciences of the United States of America 95, 12602-12607.
Svanberg, E., Zachrisson, H., Ohisson, C., Iresjo, B. M., and Lundholm, K. G. (1996). Role of insulin and IGF-1 in activation of muscle protein synthesis after oral feeding. Am J Physiol, 270, E614-620.
Tiao, G., Fagan, J., Roegner, V., Lieberman, M., Wang, J. J., Fischer, J. E., and Hasselgren, P. O. (1996). Energy-ubiquitin-dependent muscle proteolysis during sepsis in rats is regulated by glucocorticoids. Journal of Clinical Investigation 97, 339-348.
Tran, H., Brunet, A., Grenier, J. M., Datta, S. R., Fornace, A. J., Jr., DiStefano, P. S., Chiang, L. W., and Greenberg, M. E. (2002). DNA repair pathway stimulated by the forkhead transcription factor FOXO3a through the Gadd45 protein. Science 296, 530-534.
Tran, H., Brunet, A., Griffith, E. C., and Greenberg, M. E. (2003). The many forks in FOXO's road. Sci STKE 2003, RE5.
Verdu, J., Buratovich, M. A., Wilder, E. L., and Birnbaum, M. J. (1999). Cell-autonomous regulation of cell and organ growth in Drosophila by Akt/PKB. Nat Cell Biol 1, 500-506.
Wing, S. S., and Goldberg, A. L. (1993).: Glucocorticoids activate the ATP-ubiquitin-dependent proteolytic system in skeletal muscle during fasting. American Journal of Physiology 264, E668-676.
Winston, J. T., Strack, P., Beer-Romero, P., Chu, C. Y., Elledge, S. J., and Harper, J. W. (1999). The SCFbeta-TRCP-ubiquitin ligase complex associates specifically with phosphorylated destruction motifs in IkappaBalpha and beta-catenin and stimulates IkappaBalpha ubiquitination in vitro. Genes & Development 13, 270-283.
Zierath. J. R., Krook, A., and Wallberg-Henriksson, H. (2000). Insulin action resistance in human skeletal muscle. Diabetologia 43, 821-835.
   Equivalents

Those skilled in the art will recognize, or be able to ascertain using no more than routine experimentation, many equivalents to the specific embodiments of the invention described herein. Such equivalents are intended to be encompassed by the following claims.

1. A method for treating a condition related to aberrant foxo activity by modulating the phosphorylation of Foxo; stimulating Foxo phosphorylation, reducing Foxo expression; or reducing Foxo activity.

2. The method of claim 1, comprising stimulating Foxo phosphorylation.

3. The method of claim 1, comprising reducing Foxo expression.

4. The method of claim 1, comprising reducing Foxo activity.

5. The method of claim 4, wherein Foxo activity is reduced using a dominant negative mutant Foxo.

6. A method of screening for compounds that modulate Foxo activity for use in the method of claim 1, comprising contacting a cell with a test agent and measuring the effect of the test agent on Foxo activity.

7. The method of claim 6, wherein the cell is transfected with a construct comprising a atrogin promoter and a reporter gene, wherein decreased expression of the reporter gene indicates that the test compound inhibits Foxo activity.

8. The method of claim 7, wherein the atrogin promoter comprises atrogin-1 promoter.

9. A diagnostic or prognostic method of a condition related to aberrant Foxo activity involving measuring the level of Foxo phosphorylation.

10. A kit for the method of claim 9, containing an antibody that recognizes phosphorylated Foxo and a positive control sample, wherein said positive control sample originates from the muscle of a fasted patient.

11. A microarray chip comprising the plurality of cDNA sequences of genes of claim 12.

12. A method for determining the difference between levels of expression of a plurality of genes characteristic of a condition related to aberrant Foxo activity in a cell and reference levels of expression of the genes, comprising
providing RNA from a cell;
determining levels of RNA of a plurality of genes characteristic of the condition related to aberrant Foxo activity including a plurality of genes selected from the group consisting of those genes listed in Supplementary Tables 1 or 2 of Example 1 to obtain the levels of expression of the plurality of genes in the cell; and
comparing the levels of expression of the plurality of genes in the cell to a set of reference levels of expression of the genes,
to thereby determine the difference between levels of expression of the plurality of genes characteristic of the condition related to aberrant Foxo activity in the cell and reference levels of expression of the genes.

13. A diagnostic or prognostic method for a condition related to aberrant Foxo activity comprising the method of claim 12.
